(19) United States
(12) Patent Application Publication
(10) Pub. No.: US 2010/0278932 A1
(43) Pub. Date: Nov. 4, 2010

(54) POLYMER MICELLES CONTAINING SN-38 FOR THE TREATMENT OF CANCER

(75) Inventors: Kevin N. Sill, Tampa, FL (US); Habib Skaff, Tampa, FL (US); Adam Carie, Ruskin, FL (US); Jonathan Rios-Doria, Land O Lakes, FL (US); Richard Slama, Tampa, FL (US); Gregoire Cardoen, Tampa, FL (US)

Correspondence Address: CHOATE, HALL & STEWART LLP, TWO INTERNATIONAL PLACE, BOSTON, MA, 02110, US

(73) Assignee: Intezyne Technologies, Incorporated, Tampa, FL (US)

(21) Appl. No.: 12/773,702

(22) Filed: May 4, 2010

Related U.S. Application Data
(60) Provisional application No. 61/175,401, filed on May 4, 2009.

(51) Int. Cl. A61K 9/50; A61K 31/4375

(52) U.S. Cl. 424/501; 514/283

(57) ABSTRACT

The present invention provides micelles having SN-38 encapsulated therein.

### CROSS-REFERENCE TO RELATED APPLICATIONS

The present application claims priority to U.S. provisional patent application Ser. No. 61/175,401, filed May 4, 2009, the entirety of which is hereby incorporated herein by reference.

### FIELD OF THE INVENTION

The present invention relates to the field of polymer chemistry and more particularly to polymer micelles and uses thereof.

### BACKGROUND OF THE INVENTION

The development of new therapeutic agents has dramatically improved the quality of life and survival rate of patients suffering from a variety of disorders. However, drug delivery innovations are needed to improve the success rate of these treatments. Specifically, delivery systems are still needed which effectively minimize premature excretion and/or metabolism of therapeutic agents and deliver these agents specifically to diseased cells thereby reducing their toxicity to healthy cells.

Rationally-designed, nanoscopic drug carriers, or “nanovectors,” offer a promising approach to achieving these goals due to their inherent ability to overcome many biological barriers. Moreover, their multi-functionality permits the incorporation of cell-targeting groups, diagnostic agents, and a multitude of drugs in a single delivery system. Polymer micelles, formed by the molecular assembly of functional, amphiphilic block copolymers, represent one notable type of multifunctional nanovector.

Polymer micelles are particularly attractive due to their ability to deliver hydrophobic therapeutic agents. In addition, the nanoscopic size of polymeric micelles allows for passive accumulation in diseased tissues, such as solid tumors, by the enhanced permeation and retention (EPR) effect. Using appropriate surface functionality, polymer micelles are further decorated with cell-targeting groups and permeation enhancers that can actively target diseased cells and aid in cellular entry, resulting in improved cell-specific delivery.

Drug delivery vehicles are needed, which are stable to post-administration dilution, can avoid biological barriers (e.g. reticuloendothelial system (RES) uptake), and deliver drugs in response to the physiological environment encountered in diseased tissues, such as solid tumors.

### BRIEF DESCRIPTION OF THE DRAWINGS

FIG. 1 depicts a representative CMC curve for the polymer from Example 11.

FIG. 2 depicts the particle size distribution for SN-38 loaded micelles prepared with a bath sonicator.

FIG. 3 depicts the particle size distribution for SN-38 loaded micelles prepared with a probe sonicator.

FIG. 4 depicts the particle size distribution for SN-38 loaded micelles prepared with a Silverson high shear mixer.

FIG. 5 depicts the cytotoxic effects of N₃-PEG(12K)-b-P(Asp₁₀-b-P(D-Leu₂₀-co-Tyr₂₀)-Ac (Example 11) on HUVEC cells.

FIG. 6 depicts the cytotoxic effects of IT-141 in prostate cancer cell lines.

FIG. 7 depicts the cytotoxic effects of IT-141 in osteosarcoma cell lines.

FIG. 8 depicts the cytotoxic effects of IT-141 in pancreatic cancer cell line BxPC-3.

FIG. 9 depicts the cytotoxic effects of IT-141 in breast cancer cell lines.

FIG. 10 depicts the cytotoxic effects of IT-141 in breast cancer cell lines.

FIG. 11 depicts the cytotoxic effects of IT-141in colon cancer cell line Colo205.

FIG. 12 depicts the cytotoxic effects of IT-141 in colon cancer cell line HT-29.

FIG. 13 depicts the cytotoxic effects of IT-141 colon cancer cell line HCT-116.

FIG. 14 depicts the IC₅₀ (nm) values in various cancer cell lines.

FIG. 15 shows that IT-141 preferentially induces S-Phase arrest in HT-29 and MDA-MB-231 cells.

FIG. 16 shows that IT-141-1% RGD enters cells via integrins.

FIG. 17 depicts mouse weight (percent change) during an MTD Study of IT-141 and IT-141-1% RGD.

FIG. 18 depicts mouse weight (percent change) during the MTD study of IT-141 in tumor-bearing nude mice and healthy CD-1 mice.

FIG. 19 depicts the dose response reduction in HT-29 tumor volume resulting from IT-141 treatment.

FIG. 20 depicts the safety profile of IT-141 based on animal weight loss.

FIG. 21 depicts the antitumor efficacy of IT-141 comparing targeted and untargeted formulations and CPT-11 against HT-29 colon tumor xenografts.

FIG. 22 depicts the safety profile of targeted and untargeted IT-141 formulations compared to CPT-11 and polymer alone.

FIG. 23 depicts a summary of pathological findings from toxicology study.

FIG. 24 depicts the results of an antitumor efficacy study comparing targeted and untargeted formulations of IT-141 against HT-29 colon tumor xenografts at 15 mg/kg.

FIG. 25 depicts the safety profile of targeted and untargeted IT-141 formulations compared to saline.

FIG. 26 depicts the results of an antitumor efficacy study comparing targeted and untargeted formulations of IT-141 against HT-29 colon tumor xenografts at 7.5 mg/kg.

FIG. 27 depicts the results of an antitumor efficacy study comparing IT-141 formulations loaded with 11% or 4% SN-38 at equivalent mg/kg doses.

FIG. 28 depicts the dose response for reduction in HCT-116 colon tumor volume with IT-141 treatment.

FIG. 29 depicts the dose response for reduction in HT-29 colon tumor volume with IT-141-1% RGD treatment.

FIG. 30 depicts the pharmacokinetic data for SN-38 loaded polymer micelle (Example 19) in tumor bearing mice.

FIG. 31 depicts a general scheme for the preparation of IT-141 by bath sonication.

FIG. 32 depicts a general scheme for the preparation of IT-141 by probe sonication.

FIG. 33 depicts a general scheme for the preparation of IT-141 by high shear mixing.

FIG. 34 depicts a general scheme for the preparation of RGD-targeted IT-141.

FIG. 35 depicts a general scheme for the preparation of HER2-targeted IT-141.

FIG. 36 depicts a general scheme for the preparation of uPAR-targeted IT-141.

FIG. 37 depicts a general scheme for the preparation of GRP78-targeted IT-141.

FIG. 38 depicts mouse body weight during and empty micelle MTD study.

FIG. 39 depicts the SN-38 tumor accumulation for Example 19.

FIG. 40 depicts the SN-38 liver accumulation for Example 19.

FIG. 41 depicts micelle size distribution from Example 40.

FIG. 42 depicts the SN-38 plasma accumulation for Example 40.

FIG. 43 depicts the SN-38 tumor accumulation for Example 40.

FIG. 44 depicts the SN-38 liver accumulation for Example 40.

### DETAILED DESCRIPTION OF CERTAIN EMBODIMENTS OF THE INVENTION

### 1. General Description

According to one embodiment, the present invention provides a micelle comprising a multiblock copolymer having SN-38 (7-ethyl-10-hydroxycamptothecin) encapsulated therein.

The multiblock copolymer comprises a hydrophilic poly(ethylene glycol) block, a carboxylic acid-containing poly(amino acid) block, and a hydrophobic D,L-mixed poly(amino acid) block characterized in that the resulting micelle has an inner core, a carboxylic acid-containing outer core, and a hydrophilic shell. It will be appreciated that the hydrophilic poly(ethylene glycol) block corresponds to the hydrophilic shell, stabilizing carboxylic acid-containing poly(amino acid) block corresponds to the carboxylic acid-containing outer core, and the hydrophobic D,L-mixed poly(amino acid) block corresponds to the inner core.

### 2. Definitions

Compounds of this invention include those described generally above, and are further illustrated by the embodiments, sub-embodiments, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

As used herein, the term “multiblock copolymer” refers to a polymer comprising one synthetic polymer portion and two or more poly(amino acid) portions. Such multi-block copolymers include those having the format W—X′—X″, wherein W is a synthetic polymer portion and X and X′ are poly(amino acid) chains or “amino acid blocks”. In certain embodiments, the multiblock copolymers of the present invention are triblock copolymers. As described herein, one or more of the amino acid blocks may be “mixed blocks”, meaning that these blocks can contain a mixture of amino acid monomers thereby creating multiblock copolymers of the present invention. In some embodiments, the multiblock copolymers of the present invention comprise a mixed amino acid block and are tetrablock copolymers.

One skilled in the art will recognize that a monomer repeat unit is defined by parentheses depicted around the repeating monomer unit. The number (or letter representing a numerical range) on the lower right of the parentheses represents the number of monomer units that are present in the polymer chain. In the case where only one monomer represents the block (e.g. a homopolymer), the block will be denoted solely by the parentheses. In the case of a mixed block, multiple monomers comprise a single, continuous block. It will be understood that brackets will define a portion or block. For example, one block may consist of four individual monomers, each defined by their own individual set of parentheses and number of repeat units present. All four sets of parentheses will be enclosed by a set of brackets, denoting that all four of these monomers combine in random, or near random, order to comprise the mixed block. For clarity, the randomly mixed block of [BCADDCBADABCDABC] would be represented in shorthand by [(A)₄(B)₄(C)₄(D)₄].

As used herein, the term “triblock copolymer” refers to a polymer comprising one synthetic polymer portion and two poly(amino acid) portions.

As used herein, the term “inner core” as it applies to a micelle of the present invention refers to the center of the micelle formed by the hydrophobic D,L-mixed poly(amino acid) block. In accordance with the present invention, the inner core is not crosslinked. By way of illustration, in a triblock polymer of the format W—X′—X″, as described above, the inner core corresponds to the X″ block.

As used herein, the term “outer core” as it applies to a micelle of the present invention refers to the layer formed by the first poly(amino acid) block. The outer core lies between the inner core and the hydrophilic shell. In accordance with the present invention, the outer core is either crosslinkable or is cross-linked. By way of illustration, in a triblock polymer of the format W—X′—X″, as described above, the outer core corresponds to the X′ block. It is contemplated that the X′ block can be a mixed block.

As used herein, the terms “drug-loaded” and “encapsulated”, and derivatives thereof, are used interchangeably. In accordance with the present invention, a “drug-loaded” micelle refers to a micelle having a drug, or therapeutic agent, situated within the core of the micelle. In certain instances, the drug or therapeutic agent is situated at the interface between the core and the hydrophilic coronoa. This is also referred to as a drug, or therapeutic agent, being “encapsulated” within the micelle.

As used herein, the term “polymeric hydrophilic block” refers to a polymer that is not a poly(amino acid) and is hydrophilic in nature. Such hydrophilic polymers are well known in the art and include polyethyleneoxide (also referred to as polyethylene glycol or PEG), and derivatives thereof, poly(N-vinyl-2-pyrolidone), and derivatives thereof, poly(N-isopropylacrylamide), and derivatives thereof, poly(hydroxyethyl acrylate), and derivatives thereof, poly(hydroxylethyl methacrylate), and derivatives thereof, and polymers of N-(2-hydroxypropoyl)methacrylamide (HMPA) and derivatives thereof.

As used herein, the term “poly(amino acid)” or “amino acid block” refers to a covalently linked amino acid chain wherein each monomer is an amino acid unit. Such amino acid units include natural and unnatural amino acids. In certain embodiments, each amino acid unit of the optionally a crosslinkable or crosslinked poly(amino acid block)is in the L-configuration. Such poly(amino acids) include those having suitably protected functional groups. For example, amino acid monomers may have hydroxyl or amino moieties which are optionally protected by a suitable hydroxyl protecting group or a suitable amine protecting group, as appropriate. Such suitable hydroxyl protecting groups and suitable amine protecting groups are described in more detail herein, infra. As used herein, an amino acid block comprises one or more monomers or a set of two or more monomers. In certain embodiments, an amino acid block comprises one or more monomers such that the overall block is hydrophilic. In still other embodiments, amino acid blocks of the present invention include random amino acid blocks, ie blocks comprising a mixture of amino acid residues.

As used herein, the term “D,L-mixed poly(amino acid) block” refers to a poly(amino acid) block wherein the poly(amino acid) consists of a mixture of amino acids in both the D- and L-configurations. In certain embodiments, the D,L-mixed poly(amino acid) block is hydrophobic. In other embodiments, the D,L-mixed poly(amino acid) block consists of a mixture of D-configured hydrophobic amino acids and L-configured hydrophilic amino acid side-chain groups such that the overall poly(amino acid) block comprising is hydrophobic.

Exemplary poly(amino acids) include poly(benzyl glutamate), poly(benzyl aspartate), poly(L-leucine-co-tyrosine), poly(D-leucine-co-tyrosine), poly(L-phenylalanine-co-tyrosine), poly(D-phenylalanine-co-tyrosine), poly(L-leucine-coaspartic acid), poly(D-leucine-co-aspartic acid), poly(L-phenylalanine-co-aspartic acid), poly(D-phenylalanine-co-aspartic acid).

As used herein, the phrase “natural amino acid side-chain group” refers to the side-chain group of any of the 20 amino acids naturally occuring in proteins. Such natural amino acids include the nonpolar, or hydrophobic amino acids, glycine, alanine, valine, leucine isoleucine, methionine, phenylalanine, tryptophan, and proline. Cysteine is sometimes classified as nonpolar or hydrophobic and other times as polar. Natural amino acids also include polar, or hydrophilic amino acids, such as tyrosine, serine, threonine, aspartic acid (also known as aspartate, when charged), glutamic acid (also known as glutamate, when charged), asparagine, and glutamine. Certain polar, or hydrophilic, amino acids have charged side-chains. Such charged amino acids include lysine, arginine, and histidine. One of ordinary skill in the art would recognize that protection of a polar or hydrophilic amino acid side-chain can render that amino acid nonpolar. For example, a suitably protected tyrosine hydroxyl group can render that tyroine nonpolar and hydrophobic by virtue of protecting the hydroxyl group.

As used herein, the phrase “unnatural amino acid side-chain group” refers to amino acids not included in the list of 20 amino acids naturally occuring in proteins, as described above. Such amino acids include the D-isomer of any of the 20 naturally occuring amino acids. Unnatural amino acids also include homoserine, ornithine, and thyroxine. Other unnatural amino acids side-chains are well know to one of ordinary skill in the art and include unnatural aliphatic side chains. Other unnatural amino acids include modified amino acids, including those that are N-alkylated, cyclized, phosphorylated, acetylated, amidated, azidylated, labelled, and the like.

As used herein, the term “tacticity” refers to the stereochemistry of the poly(amino acid) hydrophobic block. A poly(amino acid) block consisting of a single stereoisomer (e.g. all L isomer) is referred to as “isotactic”. A poly(amino acid) consisting of a random incorporation of D and L amino acid monomers is referred to as an “atactic” polymer. A poly(amino acid) with alternating stereochemistry (e.g. . . . DLDLDL . . . ) is referred to as a “syndiotactic” polymer. Polymer tacticity is described in more detail in “Principles of Polymerization”, 3rd Ed., G. Odian, John Wiley & Sons, New York: 1991, the entire contents of which are hereby incorporated by reference.

The term “aliphatic” or “aliphatic group”, as used herein, denotes a hydrocarbon moiety that may be straight-chain (i.e., unbranched), branched, or cyclic (including fused, bridging, and spiro-fused polycyclic) and may be completely saturated or may contain one or more units of unsaturation, but which is not aromatic. Unless otherwise specified, aliphatic groups contain 1-20 carbon atoms. In some embodiments, aliphatic groups contain 1-10 carbon atoms. In other embodiments, aliphatic groups contain 1-8 carbon atoms. In still other embodiments, aliphatic groups contain 1-6 carbon atoms, and in yet other embodiments aliphatic groups contain 1-4 carbon atoms. Suitable aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as in neutron scattering experiments, as analytical tools or probes in biological assays.

As used herein, the term “detectable moiety” is used interchangeably with the term “label” and relates to any moiety capable of being detected (e.g., primary labels and secondary labels). A “detectable moiety” or “label” is the radical of a detectable compound.

“Primary” labels include radioisotope-containing moieties (e.g., moieties that contain ³²P, ³³P, ³⁵S, or ¹⁴C), mass-tags, and fluorescent labels, and are signal-generating reporter groups which can be detected without further modifications.

Other primary labels include those useful for positron emission tomography including molecules containing radioisotopes (e.g. ¹⁸F) or ligands with bound radioactive metals (e.g. ⁶²Cu). In other embodiments, primary labels are contrast agents for magnetic resonance imaging such as gadolinium, gadolinium chelates, or iron oxide (e.g Fe₃O₄ and Fe₂O₃) particles. Similarly, semiconducting nanoparticles (e.g. cadmium selenide, cadmium sulfide, cadmium telluride) are useful as fluorescent labels. Other metal nanoparticles (e.g colloidal gold) also serve as primary labels.

Unless otherwise indicated, radioisotope-containing moieties are optionally substituted hydrocarbon groups that contain at least one radioisotope. Unless otherwise indicated, radioisotope-containing moieties contain from 1-40 carbon atoms and one radioisotope. In certain embodiments, radioisotope-containing moieties contain from 1-20 carbon atoms and one radioisotope.

The terms “fluorescent label”, “fluorescent group”, “fluorescent compound”, “fluorescent dye”, and “fluorophore”, as used herein, refer to compounds or moieties that absorb light energy at a defined excitation wavelength and emit light energy at a different wavelength. Examples of fluorescent compounds include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, Texas Red-X.

### 3. Description of Exemplary Embodiments

A. SN-38 Loaded Multiblock Copolymer Micelles

The antitumor plant alkaloid camptothecin (CPT) is a broad-spectrum anticancer agent that targets DNA topoisomerase I. Although CPT has shown promising antitumor activity in vitro and in vivo, it has not been clinically used because of its low therapeutic efficacy and severe toxicity. Among CPT analogues, irinotecan hydrochloride (CPT-11) has recently been shown to be active against colorectal, lung, and ovarian cancer. CPT-11 itself is a prodrug and is converted to 7-ethyl-10-hydroxy-CPT (known as SN-38), a biologically active metabolite of CPT-11, by carboxylesterases in vivo, having the following chemical structure:

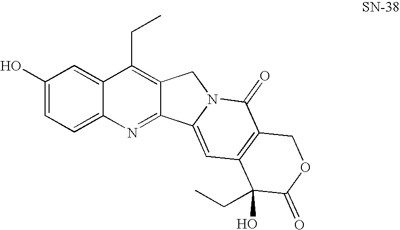

SN-38 exhibits up to 1,000-fold more potent cytotoxic activity against various cancer cells in vitro than CPT-11. Although CPT-11 is converted to SN-38 in the liver and tumor, the metabolic conversion rate is <10% of the original volume of CPT-11. In addition, the conversion of CPT-11 to SN-38 varies among patients due to inherent variations carboxylesterase activity. Thus, SN-38 has an advantage over its camptothecin precursors in that it does not require activation in vivo by the liver.

Notwishtstanding the fact that SN-38 is more effective than CPT-11 as an antineoplastic agent, SN-38 is exceedingly insoluble in aqueous solutions. Therefore, no formulation for administration of SN-38 to a patient has yet been developed. Thus, formulations are needed that improve SN-38 efficacy such that SN-38 can be used effectively in the treatment of diseases associated with cellular proliferation. Such a formulation should have suitable solubility and toxicity characteristics and will be useful in the treatment of certain proliferative diseases such as cancer.

As described generally above, the present invention provides a micelle comprising a multiblock copolymer having SN-38 (7-ethyl-10-hydroxycamptothecin) encapsulated therein.

The multiblock copolymer comprises a hydrophilic poly(ethylene glycol) block, a carboxylic acid-containing poly(amino acid) block, and a hydrophobic D,L-mixed poly(amino acid) block characterized in that the resulting micelle has an inner core, a carboxylic acid-containing outer core, and a hydrophilic shell. It will be appreciated that the hydrophilic poly(ethylene glycol) block corresponds to the hydrophilic shell, stabilizing carboxylic acid-containing poly(amino acid) block corresponds to the carboxylic acid-containing outer core, and the hydrophobic D,L-mixed poly(amino acid) block corresponds to the inner core.

Amphiphilic multiblock copolymers, as described herein, can self-assemble in aqueous solution to form nano- and micron-sized structures. In water, these amphiphilic multiblock copolymers assemble by multi-molecular micellization when present in solution above the critical micelle concentration (CMC). Without wishing to be bound by any particular theory, it is believed that the hydrophobic poly(amino acid) portion or “block” of the copolymer collapses to form the micellar core, while the hydrophilic PEG block forms a peripheral corona and imparts water solubility. In certain embodiments, the multiblock copolymers in accordance with the present invention possess distinct hydrophobic and hydrophilic segments that form micelles. In addition, these multiblock polymers optionally comprise a poly(amino acid) block which contains functionality suitable for crosslinking It will be appreciated that this functionality is found on the corresponding amino acid side-chain.

In certain embodiments, the present invention provides a micelle having SN-38 encapsulated therein, wherein said micelle comprises a multiblock copolymer which comprises:
a a hydrophilic poly(ethylene glycol) block;
a stabilizing carboxylic acid-containing poly(amino acid) block; and
a hydrophobic D,L-mixed poly(amino acid) block.

In some embodiments, the stabilizing carboxylic acid-containing poly(amino acid) block is a poly(glutamic acid) block or a poly(aspartic acid) block. In other embodiments, the stabilizing carboxylic acid-containing poly(amino acid) block is a random poly(glutamic acid-co-aspartic acid) block.

The “hydrophobic D,L-mixed poly(amino acid)” block, as described herein, consists of a mixture of D and L enantiomers to facilitate the encapsulation of hydrophobic moieties. It is well established that homopolymers and copolymers of amino acids, consisting of a single stereoisomer, may exhibit secondary structures such as the α-helix or β-sheet. See α-Aminoacid-N-Carboxy-Anhydrides and Related Heterocycles, H. R. Kricheldorf, Springer-Verlag, 1987. For example, poly(L-benzyl glutatmate) typically exhibits an a-helical conformation; however this secondary structure can be disrupted by a change of solvent or temperature (see Advances in Protein Chemistry XVI, P. Urnes and P. Doty, Academic Press, New York 1961). The secondary structure can also be disrupted by the incorporation of structurally dissimilar amino acids such as b-sheet forming amino acids (e.g. proline) or through the incorporation of amino acids with dissimilar stereochemistry (e.g. mixture of D and L stereoisomers), which results in poly(amino acids) with a random coil conformation. See Sakai, R.; Ikeda; S.; Isemura, T. Bull Chem. Soc. Japan 1969, 42, 1332-1336, Paolillo, L.; Temussi, P. A.; Bradbury, E. M.; Crane-Robinson, C. Biopolymers 1972, 11, 2043-2052, and Cho, I.; Kim, J. B.; Jung, H. J. Polymer 2003, 44, 5497-5500.

While the methods to influence secondary structure of poly(amino acids) have been known for some time, it has been suprisingly discovered that block copolymers of the present invention, possessing a random coil conformation, are particularly useful for encapsulation of hydrophobic molecules, especially SN-38, when compared to similar block copolymers possessing a helical segment. Without wishing to be bound to any particular theory, it is believed that provided block copolymers having a coil-coil conformation allow for efficient packing and loading of hydrophobic moieties within the micelle core, while the steric demands of a rod-coil conformation for a helix-containing block copolymer results in less effective encapsulation. Indeed, it has been found that encapsulation of SN-38 within a provided copolymer micelle allows for drastically increased solubility of SN-38 in water. This increased solubility allows, for the first time, administration of SN-38 to patients. Specifically, encapsulated SN-38, in accordance with the present invention, results in 2000-fold increase in solubility of SN-38 as compared to free SN-38. As used herein, the term “free SN-38” refers to SN-38 that is not encapsulated by a provided micelle in accordance with the present invention.

Remarkably, encapsulation of SN-38 within a provided polymer micelle comprised of a triblock copolymer comprising a poly(ethylene glycol) hydrophilic block, a poly(aspartic acid) outer core and a mixed [D-Leucine-co-L-Tyrosine] hydrophobic inner core, the resulting micelles exhibit greatly enhanced stability upon dilution in both aqueous media and plasma. Without wishing to be bound to any particular theory, it is believed that the resulting hydrophobic interactions are balanced to encapsulate SN-38 into the inner and/or outer core of the micelle. Specifically, it is believed that, once the SN-38 is successfully encapsulated, Van der Waals interactions between the SN-38 and the inner core bind the micelle together, allowing for increased stability upon dilution. This stability allows for an improved pharmacokinetic profile as compared to corresponding micelles encapsulating hydrophobic drugs other than SN-38.

In certain embodiments, the PEG block possesses a molecular weight of approx. 10,000 Da (225 repeat units). In other embodiments, the PEG block possesses a molecular weight of approx. 12,000 Da (270 repeat units). In yet other embodiments, the PEG block possesses a molecular weight of approx. 8,000 Da (180 repeat units). In certain embodiments, the PEG block possesses a molecular weight of approx. 20,000 Da (450 repeat units). Without wishing to be bound by theory, it is believed that this particular PEG chain length imparts adequate water-solubility to the micelles and provides relatively long in vivo circulation times.

In certain embodiments, the present invention provides a micelle, having SN-38 encapsulated therein, comprising a multiblock copolymer of formula I:

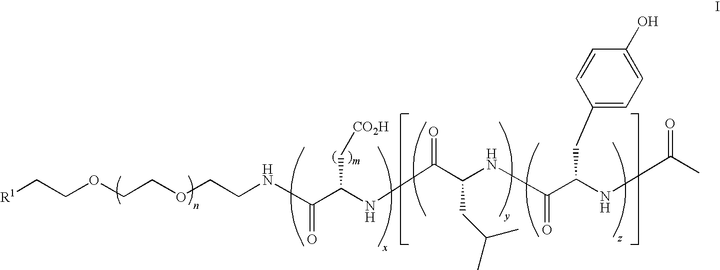

wherein:
R¹ is —OCH₃, —N₃, or

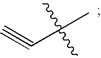

n is 110 to 450;
m is 1 or 2;
x is 3 to 50;
y is 5 to 50; and
z is 5 to 50.

In certain embodiments, the present invention provides a micelle, having SN-38 encapsulated therein, comprising a multiblock copolymer of formula I:

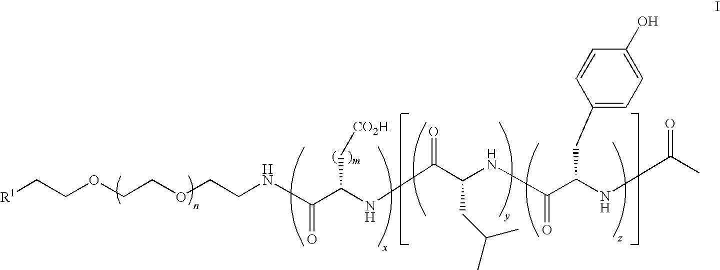

wherein:
R¹ is —N₃;
n is about 270;
m is 1;
x is about 10;
y is about 20; and
z is about 20.

In certain embodiments, the present invention provides a micelle, having SN-38 encapsulated therein, comprising a multiblock copolymer of formula I:

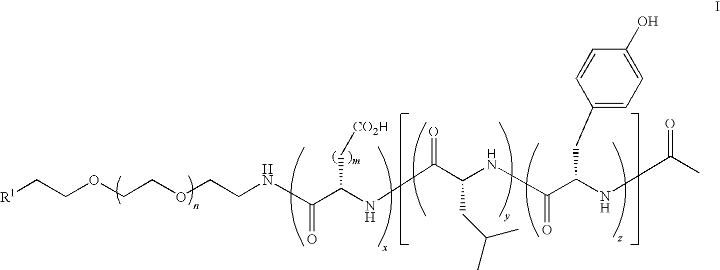

wherein:
R¹ is —OCH₃;
n is about 270;
m is 1;
x is about 10;
y is about 20; and
z is about 20.

As defined generally above, the n group of formula I is 110-450. In certain embodiments, the present invention provides compounds of formula I, as described above, wherein n is about 225. In other embodiments, n is about 270. In other embodiments, n is about 350. In other embodiments, n is about 110. In other embodiments, n is about 450. In other embodiments, n is selected from 110±10, 180±10, 225±10, 275±10, 315±10, or 450±10.

As defined generally above, the m group of formula I is 1 or 2. In some embodiments, m is 1 thereby forming a poly(aspartic acid) block. In some embodiments, m is 2 thereby forming a poly(glutamic acid) block.

In certain embodiments, the x group of formula I is about 3 to about 50. In certain embodiments, the x group of formula I is about 10. In other embodiments, x is about 20. According to yet another embodiment, x is about 15. In other embodiments, x is about 5. In other embodiments, x is selected from 5±3, 10±3, 10±5, 15±5, or 20±5.

In certain embodiments, the y group of formula I is about 5 to about 50. In certain embodiments, the y group of formula I is about 10. In other embodiments, y is about 20. According to yet another embodiment, y is about 15. In other embodiments, y is about 30. In other embodiments, y is selected from 10±3, 15±3, 17±3, 20±5, 30±5, or 40±5.

In certain embodiments, the z group of formula I is about 5 to about 50. In certain embodiments, the z group of formula I is about 10. In other embodiments, z is about 20. According to yet another embodiment, z is about 15. In other embodiments, z is about 30. In other embodiments, z is selected from 10±3, 15±3, 17±3, 20±5, 30±5, or 40±5.

In some embodiments, the R¹ group of a compound of formula I is —N₃ suitable for Click chemistry, and therefore useful for conjugating said compound to biological systems or macromolecules such as proteins, viruses, and cells, to name but a few. The Click reaction is known to proceed quickly and selectively under physiological conditions. In contrast, most conjugation reactions are carried out using the primary amine functionality on proteins (e.g. lysine or protein end-group). Because most proteins contain a multitude of lysines and arginines, such conjugation occurs uncontrollably at multiple sites on the protein. This is particularly problematic when lysines or arginines are located around the active site of an enzyme or other biomolecule. Thus, another embodiment of the present invention provides a method of conjugating the azide end group of a compound of formula I to a macromolecule via Click chemistry. Yet another embodiment of the present invention provides a macromolecule conjugated to a compound of formula I via the R¹ azide group.

In certain embodiments, the present invention provides a micelle, having SN-38 encapsulated therein, comprising a multiblock copolymer of formula II:

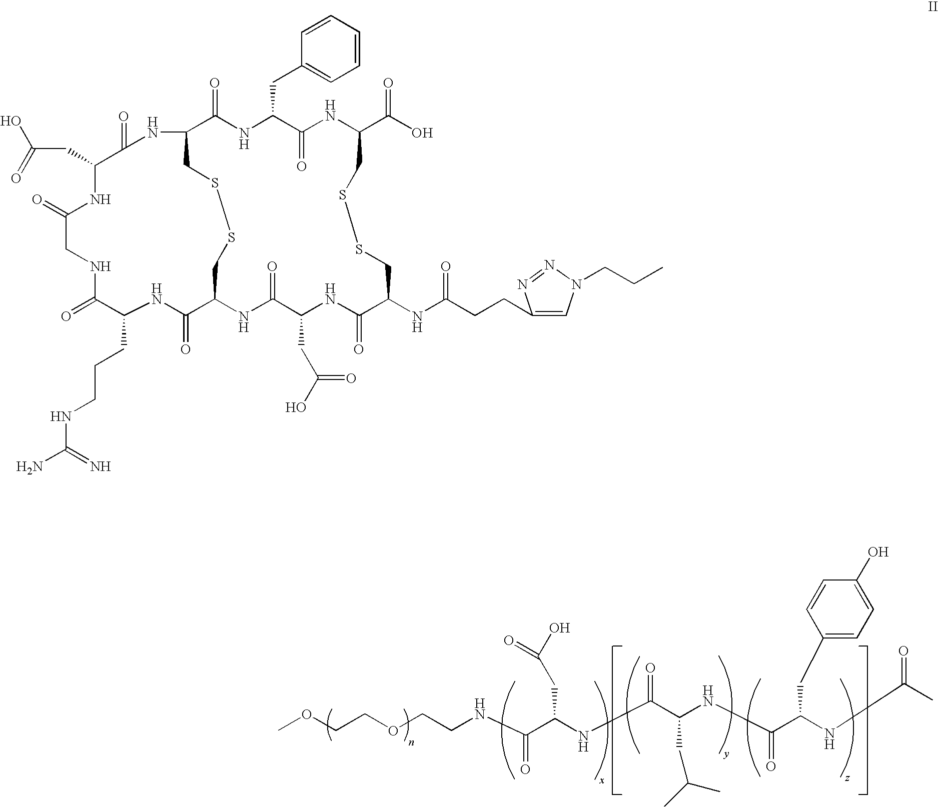

wherein:
n is 110 to 450;
x is 3 to 50;
y is 5 to 50; and
z is 5 to 50.

As defined generally above, the n group of formula II is 110-450. In certain embodiments, the present invention provides compounds of formula II, as described above, wherein n is about 225. In other embodiments, n is about 270. In other embodiments, n is about 350. In other embodiments, n is about 110. In other embodiments, n is about 450. In other embodiments, n is selected from 110±10, 180±10, 225±10, 275±10, 315±10, or 450±10.

In certain embodiments, the x group of formula II is about 3 to about 50. In certain embodiments, the x group of formula II is about 10. In other embodiments, x is about 20. According to yet another embodiment, x is about 15. In other embodiments, x is about 5. In other embodiments, x is selected from 5±3, 10±3, 10±5, 15±5, or 20±5.

In certain embodiments, the y group of formula II is about 5 to about 50. In certain embodiments, the y group of formula II is about 10. In other embodiments, y is about 20. According to yet another embodiment, y is about 15. In other embodiments, y is about 30. In other embodiments, y is selected from 10±3, 15±3, 17±3, 20±5, 30±5, or 40±5.

In certain embodiments, the z group of formula II is about 5 to about 50. In certain embodiments, the z group of formula II is about 10. In other embodiments, z is about 20. According to yet another embodiment, z is about 15. In other embodiments, z is about 30. In other embodiments, z is selected from 10±3, 15±3, 17±3, 20±5, 30±5, or 40±5.

In some embodiments, the present invention provides a micelle, having SN-38 encapsulated therein, comprising a multiblock copolymer of formula II, wherein n is about 270, x is about 10, y is about 20, and z is about 20.

In certain embodiments, the present invention provides a micelle, having SN-38 encapsulated therein, comprising a multiblock copolymer of formula I and a multiblock copolymer of formula II, wherein each of formula I and formula II are as defined above and described herein.

In some embodiments, the present invention provides a micelle, having SN-38 encapsulated therein, comprising a multiblock copolymer of formula I and a multiblock copolymer of formula II, wherein each of formula I and formula II are as defined above and described herein, wherein the ratio of Formula I to Formula II is between about 1000:1 and about 1:1. In other embodiments, the ratio is about 1000:1, about 100:1, about 50:1, about 33:1, about 25:1, about 20:1, about 10:1, about 5:1, or about 4:1. In yet other embodiments, the ratio is between about 100:1 and about 25:1.

In certain embodiments, the present invention provides a micelle, having SN-38 encapsulated therein, comprising a multiblock copolymer of formula III:

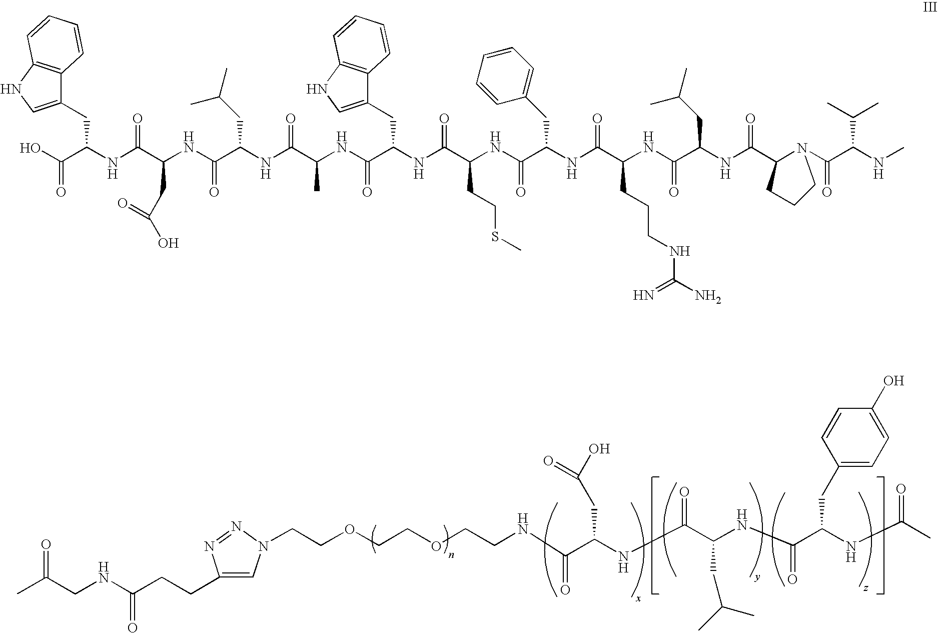

wherein:
n is 110 to 450;
x is 3 to 50;
y is 5 to 50; and
z is 5 to 50.

As defined generally above, the n group of formula III is 110-450. In certain embodiments, the present invention provides compounds of formula III, as described above, wherein n is about 225. In other embodiments, n is about 270. In other embodiments, n is about 350. In other embodiments, n is about 110. In other embodiments, n is about 450. In other embodiments, n is selected from 110±10, 180±10, 225±10, 275±10, 315±10, or 450±10.

In certain embodiments, the x group of formula III is about 3 to about 50. In certain embodiments, the x group of formula III is about 10. In other embodiments, x is about 20. According to yet another embodiment, x is about 15. In other embodiments, x is about 5. In other embodiments, x is selected from 5±3, 10±3, 10±5, 15±5, or 20±5.

In certain embodiments, the y group of formula III is about 5 to about 50. In certain embodiments, the y group of formula III is about 10. In other embodiments, y is about 20. According to yet another embodiment, y is about 15. In other embodiments, y is about 30. In other embodiments, y is selected from 10±3, 15±3, 17±3, 20±5, 30±5, or 40±5.

In certain embodiments, the z group of formula III is about 5 to about 50. In certain embodiments, the z group of formula III is about 10. In other embodiments, z is about 20. According to yet another embodiment, z is about 15. In other embodiments, z is about 30. In other embodiments, z is selected from 10±3, 15±3, 17±3, 20±5, 30±5, or 40±5.

In certain embodiments, the present invention provides a micelle, having SN-38 encapsulated therein, comprising a multiblock copolymer of formula III wherein wherein n is about 270, x is about 10, y is about 20, and z is about 20.

In some embodiments, the present invention provides a micelle, having SN-38 encapsulated therein, comprising a multiblock copolymer of formula I and a multiblock copolymer of formula III, wherein each of formula I and formula III are as defined above and described herein, wherein the ratio of Formula I to Formula III is between about 1000:1 and about 1:1. In other embodiments, the ratio is about 1000:1, about 100:1, about 50:1, about 33:1, about 25:1, about 20:1, about 10:1, about 5:1, or about 4:1. In yet other embodiments, the ratio is between about 100:1 and about 25:1.

In certain embodiments, the present invention provides a micelle, having SN-38 encapsulated therein, comprising a multiblock copolymer of formula IV:

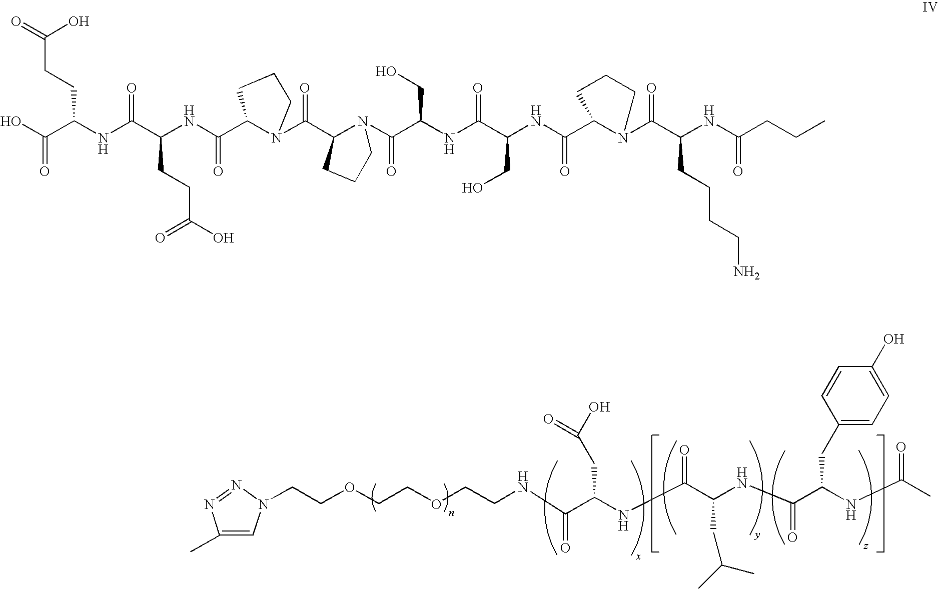

wherein:
n is 110 to 450;
x is 3 to 50;
y is 5 to 50; and
z is 5 to 50.

As defined generally above, the n group of formula IV is 110-450. In certain embodiments, the present invention provides compounds of formula IV, as described above, wherein n is about 225. In other embodiments, n is about 270. In other embodiments, n is about 350. In other embodiments, n is about 110. In other embodiments, n is about 450. In other embodiments, n is selected from 110±10, 180±10, 225±10, 275±10, 315±10, or 450±10.

In certain embodiments, the x group of formula IV is about 3 to about 50. In certain embodiments, the x group of formula IV is about 10. In other embodiments, x is about 20. According to yet another embodiment, x is about 15. In other embodiments, x is about 5. In other embodiments, x is selected from 5±3, 10±3, 10±5, 15±5, or 20±5.

In certain embodiments, the y group of formula IV is about 5 to about 50. In certain embodiments, the y group of formula IV is about 10. In other embodiments, y is about 20. According to yet another embodiment, y is about 15. In other embodiments, y is about 30. In other embodiments, y is selected from 10±3, 15±3, 17±3, 20±5, 30±5, or 40±5.

In certain embodiments, the z group of formula IV is about 5 to about 50. In certain embodiments, the z group of formula IV is about 10. In other embodiments, z is about 20. According to yet another embodiment, z is about 15. In other embodiments, z is about 30. In other embodiments, z is selected from 10±3, 15±3, 17±3, 20±5, 30±5, or 40±5.

In certain embodiments, the present invention provides a micelle, having SN-38 encapsulated therein, comprising a multiblock copolymer of formula IV wherein wherein n is about 270, x is about 10, y is about 20, and z is about 20.

In some embodiments, the present invention provides a micelle, having SN-38 encapsulated therein, comprising a multiblock copolymer of formula I and a multiblock copolymer of formula IV, wherein each of formula I and formula IV are as defined above and described herein, wherein the ratio of Formula I to Formula IV is between about 1000:1 and about 1:1. In other embodiments, the ratio is about 1000:1, about 100:1, about 50:1, about 33:1, about 25:1, about 20:1, about 10:1, about 5:1, or about 4:1. In yet other embodiments, the ratio is between about 100:1 and about 25:1.

In certain embodiments, the present invention provides a micelle, having SN-38 encapsulated therein, comprising a multiblock copolymer of formula V:

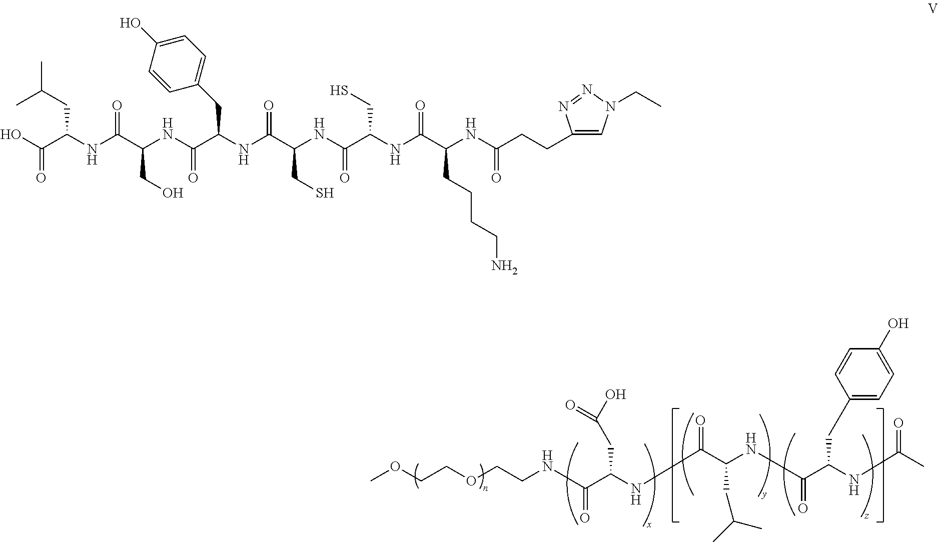

wherein:
n is 110 to 450;
x is 3 to 50;
y is 5 to 50; and
z is 5 to 50.

As defined generally above, the n group of formula V is 110-450. In certain embodiments, the present invention provides compounds of formula V, as described above, wherein n is about 225. In other embodiments, n is about 270. In other embodiments, n is about 350. In other embodiments, n is about 110. In other embodiments, n is about 450. In other embodiments, n is selected from 110±10, 180±10, 225±10, 275±10, 315±10, or 450±10.

In certain embodiments, the x group of formula V is about 3 to about 50. In certain embodiments, the x group of formula V is about 10. In other embodiments, x is about 20. According to yet another embodiment, x is about 15. In other embodiments, x is about 5. In other embodiments, x is selected from 5±3, 10±3, 10±5, 15±5, or 20±5.

In certain embodiments, the y group of formula V is about 5 to about 50. In certain embodiments, the y group of formula V is about 10. In other embodiments, y is about 20. According to yet another embodiment, y is about 15. In other embodiments, y is about 30. In other embodiments, y is selected from 10±3, 15±3, 17±3, 20±5, 30±5, or 40±5.

In certain embodiments, the z group of formula V is about 5 to about 50. In certain embodiments, the z group of formula V is about 10. In other embodiments, z is about 20. According to yet another embodiment, z is about 15. In other embodiments, z is about 30. In other embodiments, z is selected from 10±3, 15±3, 17±3, 20±5, 30±5, or 40±5.

In certain embodiments, the present invention provides a micelle, having SN-38 encapsulated therein, comprising a multiblock copolymer of formula V wherein wherein n is about 270, x is about 10, y is about 20, and z is about 20.

In some embodiments, the present invention provides a micelle, having SN-38 encapsulated therein, comprising a multiblock copolymer of formula I and a multiblock copolymer of formula V, wherein each of formula I and formula V are as defined above and described herein, wherein the ratio of Formula I to Formula V is between about 1000:1 and about 1:1. In other embodiments, the ratio is about 1000:1, about 100:1, about 50:1, about 33:1, about 25:1, about 20:1, about 10:1, about 5:1, or about 4:1. In yet other embodiments, the ratio is between about 100:1 and about 25:1.

In certain embodiments, the present invention provides a micelle, having SN-38 encapsulated therein, comprising a multiblock copolymer of formula VI:

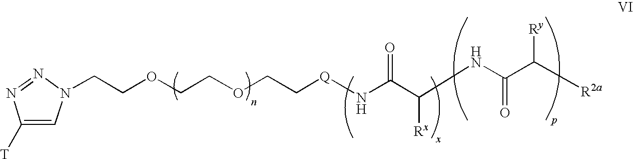

wherein:
n is 10-2500;
x is 0 to 1000;
p is 2 to 1000;
R^{x} is a natural or unnatural amino acid side-chain group that is capable of crosslinking;
R^{y} forms a hydrophobic D,L-mixed poly(amino acid) block;
Q is a valence bond or a bivalent, saturated or unsaturated, straight or branched C₁₋₁₂ hydrocarbon chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
   -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
R^{2a} is a mono-protected amine, a di-protected amine, —N(R⁴)₂, —NR⁴C(O)R⁴, —NR⁴C(O)N(R⁴)₂, —NR⁴C(O)OR⁴, or —NR⁴SO₂R⁴;
each R⁴ is independently hydrogen or an optionally substituted group selected from aliphatic, a 5-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety, or:
   two R⁴ on the same nitrogen atom are taken together with said nitrogen atom to form an optionally substituted 4-7 membered saturated, partially unsaturated, or aryl ring having 1-4 heteroatoms independently selected from nitrogen, oxygen, or sulfur, and
T is a targeting group moiety.

In certain embodiments, the p group of formula VI is about 5 to about 500. In certain embodiments, the p group of formula VI is about 10 to about 250. In other embodiments, p is about 10 to about 50. According to yet another embodiment, p is about 15 to about 40. In other embodiments, p is about 20 to about 40. According to yet another embodiment, p is about 50 to about 75. According to other embodiments, x and p are independently about 10 to about 100.

In some embodiments, x is 0. In certain embodiments, x is 5-50. In other embodiments, x is 5-25. In certain embodiments, p is 5-50. In other embodiments, p is 5-10. In other embodiments, p is 10-20. In certain embodiments, x and p add up to about 30 to about 60. In still other embodiments, x is 1-20 repeat units and p is 10-50 repeat units. In certain embodiments, the x group of formula VI is about 3 to about 50. In certain embodiments, the x group of formula VI is about 10. In other embodiments, x is about 20. According to yet another embodiment, x is about 15. In other embodiments, x is about 5. In other embodiments, x is selected from 5±3, 10±3, 10±5, 15±5, or 20±5.

As defined generally above, the Q group of formula VI is a valence bond or a bivalent, saturated or unsaturated, straight or branched C₁₋₁₂ hydrocarbon chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur. In certain embodiments, Q is a valence bond. In other embodiments, Q is a bivalent, saturated C₁₋₁₂ alkylene chain, wherein 0-6 methylene units of Q are independently replaced by -Cy-, —O—, —NH—, —S—, —OC(O)—, —C(O)O—, or —C(O)—, wherein -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur.

In certain embodiments, the Q group of formula VI is -Cy- (i.e. a C₁ alkylene chain wherein the methylene unit is replaced by -Cy-), wherein -Cy- is an optionally substituted 5-8 membered bivalent, saturated, partially unsaturated, or aryl ring having 0-4 heteroatoms independently selected from nitrogen, oxygen, or sulfur. According to one aspect of the present invention, -Cy- is an optionally substituted bivalent aryl group. According to another aspect of the present invention, -Cy- is an optionally substituted bivalent phenyl group. In other embodiments, -Cy- is an optionally substituted 5-8 membered bivalent, saturated carbocyclic ring. In still other embodiments, -Cy- is an optionally substituted 5-8 membered bivalent, saturated heterocyclic ring having 1-2 heteroatoms independently selected from nitrogen, oxygen, or sulfur. Exemplary -Cy- groups include bivalent rings selected from phenyl, pyridyl, pyrimidinyl, cyclohexyl, cyclopentyl, or cyclopropyl.

In certain embodiments, the R^{x} group of formula VI is a crosslinkable amino acid side-chain group. Such crosslinkable amino acid side-chain groups include tyrosine, serine, cysteine, threonine, aspartic acid (also known as aspartate, when charged), glutamic acid (also known as glutamate, when charged), asparagine, histidine, lysine, arginine, glutamine, or a benzimidazole-functionalized amino acid.

As defined above, the R^{x} group of formula VI is a natural or unnatural amino acid side-chain group capable of forming cross-links. It will be appreciated that a variety of amino acid side-chain functional groups are capable of such cross-linking, including, but not limited to, carboxylate, hydroxyl, thiol, and amino groups. Examples of R^{x} moieties having functional groups capable of forming cross-links include a glutamic acid side-chain, —CH₂C(O)OH, an aspartic acid side-chain, —CH₂CH₂C(O)OH, a cystein side-chain, —CH₂SH, a serine side-chain, —CH₂OH, an aldehyde containing side-chain, —CH₂C(O)H, a lysine side-chain, —(CH₂)₄NH₂, an arginine side-chain, —(CH₂)₃NHC(═NH)NH₂, a histidine side-chain, —CH₂-imidazol-4-yl. In some embodiments, R^{x} is a glutamic acid side chain. In other embodiments, R^{x} is an aspartic acid side chain. In still other embodiments, R^{x} is a histidine side-chain.

As defined above, the R^{y} group of formula VI forms a hydrophobic D,L-mixed amino acid block. Such hydrophobic amino acid side-chain groups include a suitably protected tyrosine side-chain, a suitably protected serine side-chain, a suitably protected threonine side-chain, phenylalanine, alanine, valine, leucine, tryptophan, proline, benzyl and alkyl glutamates, or benzyl and alkyl aspartates or mixtures thereof. One of ordinary skill in the art would recognize that protection of a polar or hydrophilic amino acid side-chain can render that amino acid nonpolar. For example, a suitably protected tyrosine hydroxyl group can render that tyrosine nonpolar and hydrophobic by virtue of protecting the hydroxyl group. Suitable protecting groups for the hydroxyl, amino, and thiol, and carboxylate functional groups of R^{x} and R^{y} are as described herein.

In other embodiments, the R^{y} group of formula VI consists of a mixture of D-hydrophobic and L-hydrophilic amino acid side-chain groups such that the overall poly(amino acid) block comprising R^{y} is hydrophobic and is a mixture of D- and L-configured amino acids. Such mixtures of amino acid side-chain groups include L-tyrosine and D-leucine, L-tyrosine and D-phenylalanine, L-serine and D-phenylalanine, L-aspartic acid and D-phenylalanine, L-glutamic acid and D-phenylalanine, L-tyrosine and D-benzyl glutamate, L-tyrosine and D-benzyl aspartate, L-serine and D-benzyl glutamate, L-serine and D-benzyl aspartate, L-aspartic acid and D-benzyl glutamate, L-aspartic acid and D-benzyl aspartate, L-glutamic acid and D-benzyl glutamate, L-glutamic acid and D-benzyl aspartate, L-aspartic acid and D-leucine, and L-glutamic acid and D-leucine. Ratios (D-hydrophobic to L-hydrophilic) of such amino acid combinations can range between 5-95 mol %.

In certain embodiments, the R^{y} group of formula VI consists of a mixture of D-hydrophobic and L-hydrophobic amino acids. Such mixtures include D-benzyl glutamate and L-benzyl glutamate, D-benzyl aspartate and L-benzyl aspartate, D-benzyl aspartate and L-benzyl glutamate, or D-benzyl glutamate and L-benzyl aspartate.

As defined generally above, the R^{2a} group of formula VI is a mono-protected amine, a di-protected amine, —NHR⁴, —N(R⁴)₂, —NHC(O)R⁴, —NR⁴C(O)R⁴, —NHC(O)NHR⁴, —NHC(O)N(R⁴)₂, —NR⁴C(O)NHR⁴, —NR⁴C(O)N(R⁴)₂, —NHC(O)OR⁴, —NR⁴C(O)OR⁴, —NHSO₂R⁴, or —NR⁴SO₂R⁴, wherein each R⁴ is independently an optionally substituted group selected from aliphatic, a 5-8 membered saturated, partially unsaturated, or aryl ring having 0-4 heteroatoms independently selected from nitrogen, oxygen, or sulfur, an 8-10-membered saturated, partially unsaturated, or aryl bicyclic ring having 0-5 heteroatoms independently selected from nitrogen, oxygen, or sulfur, or a detectable moiety, or two R⁴ on the same nitrogen atom are taken together with said nitrogen atom to form an optionally substituted 4-7 membered saturated, partially unsaturated, or aryl ring having 1-4 heteroatoms independently selected from nitrogen, oxygen, or sulfur.

In certain embodiments, the R^{2a} group of formula VI is —NHR⁴ or —N(R⁴)₂ wherein each R⁴ is an optionally substituted aliphatic group. One exemplary R⁴ group is 5-norbornen-2-yl-methyl. According to yet another aspect of the present invention, the R^{2a} group of formula I is —NHR⁴ wherein R⁴ is a C₁₋₆ aliphatic group substituted with N₃. Examples include —CH₂N₃. In some embodiments, R⁴ is an optionally substituted C₁₋₆ alkyl group. Examples include methyl, ethyl, propyl, butyl, pentyl, hexyl, 2-(tetrahydropyran-2-yloxy)ethyl, pyridin-2-yldisulfanylmethyl, methyldisulfanylmethyl, (4-acetylenylphenyl)methyl, 3-(methoxycarbonyl)-prop-2-ynyl, methoxycarbonylmethyl, 2-(N-methyl-N-(4-acetylenylphenyl)carbonylamino)ethyl, 2-phthalimidoethyl, 4-bromobenzyl, 4-chlorobenzyl, 4-fluorobenzyl, 4-iodobenzyl, 4-propargyloxybenzyl, 2-nitrobenzyl, 4-(bis-4-acetylenylbenzyl)aminomethyl-benzyl, 4-propargyloxy-benzyl, 4-dipropargylamino-benzyl, 4-(2-propargyloxy-ethyldisulfanyl)benzyl, 2-propargyloxy-ethyl, 2-propargyldisulfanyl-ethyl, 4-propargyloxy-butyl, 2-(N-methyl-N-propargylamino)ethyl, and 2-(2-dipropargylaminoethoxy)-ethyl. In other embodiments, R⁴ is an optionally substituted C₂₋₆ alkenyl group. Examples include vinyl, allyl, crotyl, 2-propenyl, and but-3-enyl. When R⁴ group is a substituted aliphatic group, suitable substituents on R⁴ include N₃, CN, and halogen. In certain embodiments, R⁴ is —CH₂CN, —CH₂CH₂CN, —CH₂CH(OCH₃)₂, 4-(bisbenzyloxymethyl)phenylmethyl, and the like.

According to another aspect of the present invention, the R^{2a} group of formula VI is —NHR⁴ wherein R⁴ is an optionally substituted C₂₋₆ alkynyl group. Examples include —CC≡CH, —CH₂C≡CH, —CH₂C≡CCH₃, and —CH₂CH₂C≡CH.

In certain embodiments, the R^{2a} group of formula VI is —NHR⁴ wherein R⁴ is an optionally substituted 5-8-membered aryl ring. In certain embodiments, R⁴ is optionally substituted phenyl or optionally substituted pyridyl. Examples include phenyl, 4-t-butoxycarbonylaminophenyl, 4-azidomethylphenyl, 4-propargyloxyphenyl, 2-pyridyl, 3-pyridyl, and 4-pyridyl. In certain embodiments, R^{2a} is 4-t-butoxycarbonylaminophenylamino, 4-azidomethylphenamino, or 4-prop argyloxyphenylamino.

In certain embodiments, the R^{2a} group of formula VI is —NHR⁴ wherein R⁴ is an optionally substituted phenyl ring. Suitable substituents on the R⁴ phenyl ring include halogen; —(CH₂)₀₋₄R^{o}; —(CH₂)₀₋₄OR^{o}; —(CH₂)₀₋₄CH(OR^{o})₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —(CH₂)₀₋₄SC(O)R^{o}; —(CH₂)₀₋₄C(O)NR^{o}₂; —C(S)NR^{o}₂; —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)20R^{o}; —(CH₂)₀₋₄OS(O)₂R^{o}; —S(O)₂NR^{o}₂; —(CH₂)₀₋₄S(O)R^{o}; —N(R^{o})S(O)₂NR^{o}₂; —N(R^{o})S(O)₂R^{o}; —N(OR^{o})R^{o}; —C(NH)NR^{o}₂; —P(O)₂R^{o}; —P(O)R^{o}₂; —OP(O)R^{o}₂; SiR^{o}₃; wherein each independent occurrence of R^{o} is as defined herein supra. In other embodiments, the R^{2a} group of formula I is —NHR⁴ wherein R⁴ is phenyl substituted with one or more optionally substituted C₁₋₆ aliphatic groups. In still other embodiments, R⁴ is phenyl substituted with vinyl, allyl, acetylenyl, —CH₂N₃, —CH₂CH₂N₃, —CH₂C≡CCH₃, or —CH₂C≡CH.

In certain embodiments, the R^{2a} group of formula VI is —NHR⁴ wherein R⁴ is phenyl substituted with N₃, N(R^{o})₂, CO₂R^{o}, or C(O)R^{o} wherein each R^{o} is independently as defined herein supra.

In certain embodiments, the R^{2a} group of formula VI is —N(R⁴)₂ wherein each R⁴ is independently an optionally substituted group selected from aliphatic, phenyl, naphthyl, a 5-6 membered aryl ring having 1-4 heteroatoms independently selected from nitrogen, oxygen, or sulfur, or a 8-10 membered bicyclic aryl ring having 1-5 heteroatoms independently selected from nitrogen, oxygen, or sulfur, or a detectable moiety.

In other embodiments, the R^{2a} group of formula VI is —N(R⁴)₂ wherein the two R⁴ groups are taken together with said nitrogen atom to form an optionally substituted 4-7 membered saturated, partially unsaturated, or aryl ring having 1-4 heteroatoms independently selected from nitrogen, oxygen, or sulfur. According to another embodiment, the two R⁴ groups are taken together to form a 5-6-membered saturated or partially unsaturated ring having one nitrogen wherein said ring is substituted with one or two oxo groups. Such R^{2a} groups include, but are not limited to, phthalimide, maleimide and succinimide.

In certain embodiments, the R^{2a} group of formula VI is a mono-protected or di-protected amino group. In certain embodiments R^{2a} is a mono-protected amine. In certain embodiments R^{2a} is a mono-protected amine selected from aralkylamines, carbamates, allyl amines, or amides. Exemplary mono-protected amino moieties include t-butyloxycarbonylamino, ethyloxycarbonylamino, methyloxycarbonylamino, trichloroethyloxycarbonylamino, allyloxycarbonylamino, benzyloxocarbonylamino, allylamino, benzylamino, fluorenylmethylcarbonyl, formamido, acetamido, chloroacetamido, dichloroacetamido, trichloroacetamido, phenylacetamido, trifluoroacetamido, benzamido, and t-butyldiphenylsilylamino. In other embodiments R^{2a} is a di-protected amine. Exemplary di-protected amino moieties include di-benzylamino, di-allylamino, phthalimide, maleimido, succinimido, pyrrolo, 2,2,5,5-tetramethyl-[1,2,5]azadisilolidino, and azido. In certain embodiments, the R^{2a} moiety is phthalimido. In other embodiments, the R^{2a} moiety is mono- or di-benzylamino or mono- or di-allylamino.

As defined generally above, the T group of formula VI is a targeting group moiety. Targeting groups are well known in the art and include those described in International application publication number WO 2008/134761, published Nov. 6, 2008, the entirety of which is hereby incorporated by reference. In some embodiments, the T targeting group is a moiety selected from folate, a Her-2 binding peptide, a urokinase-type plasminogen activator receptor (uPAR) antagonist, a CXCR4 chemokine receptor antagonist, a GRP78 peptide antagonist, an RGD peptide, an RGD cyclic peptide, a luteinizing hormone-releasing hormone (LHRH) antagonist peptide, an aminopeptidase targeting peptide, a brain homing peptide, a kidney homing peptide, a heart homing peptide, a gut homing peptide, an integrin homing peptide, an angiogencid tumor endothelium homing peptide, an ovary homing peptide, a uterus homing peptide, a sperm homing peptide, a microglia homing peptide, a synovium homing peptide, a urothelium homing peptide, a prostate homing peptide, a lung homing peptide, a skin homing peptide, a retina homing peptide, a pancreas homing peptide, a liver homing peptide, a lymph node homing peptide, an adrenal gland homing peptide, a thyroid homing peptide, a bladder homing peptide, a breast homing peptide, a neuroblastoma homing peptide, a lymphona homing peptide, a muscle homing peptide, a wound vasculature homing peptide, an adipose tissue homing peptide, a virus binding peptide, or a fusogenic peptide. Such targeting groups are well known in the art and are described in detail in WO 2008/134761.

In some embodiments, the T targeting group is a moiety selected from a tumor homing group, a prostate specific membrane antigen homing peptide, an aminopeptidate N homing peptide, a Her-2 homing peptide, a colon cancer homing peptide, a VEGFR1 homing peptide, or a CXCR4 homing peptide.

In certain embodiments, the present invention provides a micelle, having SN-38 encapsulated therein, comprising a multiblock copolymer of formula VI, as defined above and described herein.

In some embodiments, the present invention provides a micelle, having SN-38 encapsulated therein, comprising a multiblock copolymer of formula I and a multiblock copolymer of formula VI, wherein each of formula I and formula VI are as defined above and described herein, wherein the ratio of Formula I to Formula VI is between about 1000:1 and about 1:1. In other embodiments, the ratio is about 1000:1, about 100:1, about 50:1, about 33:1, about 25:1, about 20:1, about 10:1, about 5:1, or about 4:1. In yet other embodiments, the ratio is between about 100:1 and about 25:1.

In certain embodiments, the present invention provides a micelle, having SN-38 encapsulated therein, comprising a multiblock copolymer of formula VII:

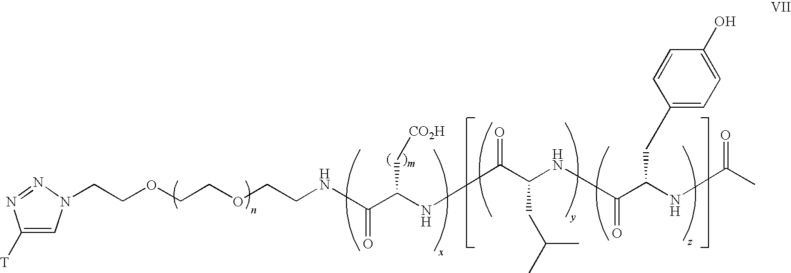

wherein:
T is a targeting group moiety;
n is 110 to 450;
m is 1 or 2;
x is 3 to 50;
y is 5 to 50; and
z is 5 to 50.

As defined generally above, the n group of formula VII is 110-450. In certain embodiments, the present invention provides compounds of formula VII, as described above, wherein n is about 225. In other embodiments, n is about 270. In other embodiments, n is about 350. In other embodiments, n is about 110. In other embodiments, n is about 450. In other embodiments, n is selected from 110±10, 180±10, 225±10, 275±10, 315±10, or 450±10.

As defined generally above, the m group of formula VII is 1 or 2. In some embodiments, m is 1 thereby forming a poly(aspartic acid) block. In some embodiments, m is 2 thereby forming a poly(glutamic acid) block.

In certain embodiments, the x group of formula VII is about 3 to about 50. In certain embodiments, the x group of formula VII is about 10. In other embodiments, x is about 20. According to yet another embodiment, x is about 15. In other embodiments, x is about 5. In other embodiments, x is selected from 5±3, 10±3, 10±5, 15±5, or 20±5.

In certain embodiments, the y group of formula VII is about 5 to about 50. In certain embodiments, the y group of formula VII is about 10. In other embodiments, y is about 20. According to yet another embodiment, y is about 15. In other embodiments, y is about 30. In other embodiments, y is selected from 10±3, 15±3, 17±3, 20±5, 30±5, or 40±5.

In certain embodiments, the z group of formula VII is about 5 to about 50. In certain embodiments, the z group of formula VII is about 10. In other embodiments, z is about 20. According to yet another embodiment, z is about 15. In other embodiments, z is about 30. In other embodiments, z is selected from 10±3, 15±3, 17±3, 20±5, 30±5, or 40±5.

In certain embodiments, the present invention provides a multiblock copolymer of formula VII wherein wherein n is about 270, x is about 10, y is about 20, and z is about 20.

In some embodiments, the T targeting group moiety of formula VII is a moiety selected from folate, a Her-2 binding peptide, a urokinase-type plasminogen activator receptor (uPAR) antagonist, a CXCR4 chemokine receptor antagonist, a GRP78 peptide antagonist, an RGD peptide, an RGD cyclic peptide, a luteinizing hormone-releasing hormone (LHRH) antagonist peptide, an aminopeptidase targeting peptide, a brain homing peptide, a kidney homing peptide, a heart homing peptide, a gut homing peptide, an integrin homing peptide, an angiogencid tumor endothelium homing peptide, an ovary homing peptide, a uterus homing peptide, a sperm homing peptide, a microglia homing peptide, a synovium homing peptide, a urothelium homing peptide, a prostate homing peptide, a lung homing peptide, a skin homing peptide, a retina homing peptide, a pancreas homing peptide, a liver homing peptide, a lymph node homing peptide, an adrenal gland homing peptide, a thyroid homing peptide, a bladder homing peptide, a breast homing peptide, a neuroblastoma homing peptide, a lymphona homing peptide, a muscle homing peptide, a wound vasculature homing peptide, an adipose tissue homing peptide, a virus binding peptide, or a fusogenic peptide. Such targeting groups are well known in the art and are described in detail in WO 2008/134761.

In some embodiments, the T targeting group is a moiety selected from a tumor homing group, a prostate specific membrane antigen homing peptide, an aminopeptidate N homing peptide, a Her-2 homing peptide, a colon cancer homing peptide, a VEGFR1 homing peptide, or a CXCR4 homing peptide.

In certain embodiments, the present invention provides a micelle, having SN-38 encapsulated therein, comprising a multiblock copolymer of formula VII, as defined above and described herein.

In some embodiments, the present invention provides a micelle, having SN-38 encapsulated therein, comprising a multiblock copolymer of formula I and a multiblock copolymer of formula VII, wherein each of formula I and formula VII are as defined above and described herein, wherein the ratio of Formula I to Formula VII is between about 1000:1 and about 1:1. In other embodiments, the ratio is about 1000:1, about 100:1, about 50:1, about 33:1, about 25:1, about 20:1, about 10:1, about 5:1, or about 4:1. In yet other embodiments, the ratio is between about 100:1 and about 25:1.

In another embodiment, the present invention provides a micelle, having SN-38 encapsulated therein, comprising a multiblock copolymer of formula I, and two or more multiblock copolymers selected from any of formula II, formula III, formula V, formula VI, or formula VII.

In certain embodiments, the present invention provides a micelle, having SN-38 encapsulated therein, comprising a multiblock copolymer of formula VIII:

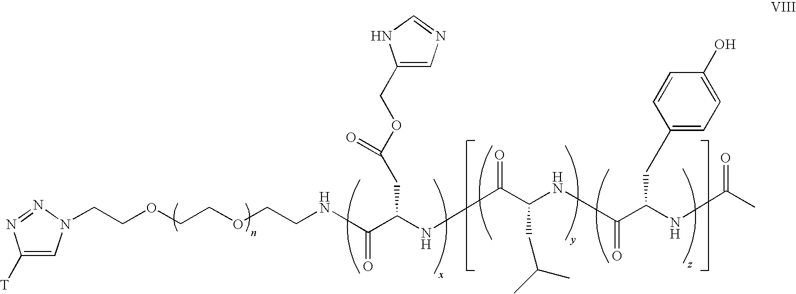

wherein:
T is a targeting group moiety;
n is 110 to 450;
x is 3 to 50;
y is 5 to 50;
z is 5 to 50.

As defined generally above, the n group of formula VIII is 110-450. In certain embodiments, the present invention provides compounds of formula VIII, as described above, wherein n is about 225. In other embodiments, n is about 270. In other embodiments, n is about 350. In other embodiments, n is about 110. In other embodiments, n is about 450. In other embodiments, n is selected from 110±10, 180±10, 225±10, 275±10, 315±10, or 450±10.

In certain embodiments, the x group of formula VIII is about 3 to about 50. In certain embodiments, the x group of formula VIII is about 10. In other embodiments, x is about 20. According to yet another embodiment, x is about 15. In other embodiments, x is about 5. In other embodiments, x is selected from 5±3, 10±3, 10±5, 15±5, or 20±5.

In certain embodiments, the y group of formula VIII is about 5 to about 50. In certain embodiments, the y group of formula VIII is about 10. In other embodiments, y is about 20. According to yet another embodiment, y is about 15. In other embodiments, y is about 30. In other embodiments, y is selected from 10±3, 15±3, 17±3, 20±5, 30±5, or 40±5.

In certain embodiments, the z group of formula VIII is about 5 to about 50. In certain embodiments, the z group of formula VIII is about 10. In other embodiments, z is about 20. According to yet another embodiment, z is about 15. In other embodiments, z is about 30. In other embodiments, z is selected from 10±3, 15±3, 17±3, 20±5, 30±5, or 40±5.

In some embodiments, the T targeting group moiety of formula VIII is a moiety selected from folate, a Her-2 binding peptide, a urokinase-type plasminogen activator receptor (uPAR) antagonist, a CXCR4 chemokine receptor antagonist, a GRP78 peptide antagonist, an RGD peptide, an RGD cyclic peptide, a luteinizing hormone-releasing hormone (LHRH) antagonist peptide, an aminopeptidase targeting peptide, a brain homing peptide, a kidney homing peptide, a heart homing peptide, a gut homing peptide, an integrin homing peptide, an angiogencid tumor endothelium homing peptide, an ovary homing peptide, a uterus homing peptide, a sperm homing peptide, a microglia homing peptide, a synovium homing peptide, a urothelium homing peptide, a prostate homing peptide, a lung homing peptide, a skin homing peptide, a retina homing peptide, a pancreas homing peptide, a liver homing peptide, a lymph node homing peptide, an adrenal gland homing peptide, a thyroid homing peptide, a bladder homing peptide, a breast homing peptide, a neuroblastoma homing peptide, a lymphona homing peptide, a muscle homing peptide, a wound vasculature homing peptide, an adipose tissue homing peptide, a virus binding peptide, or a fusogenic peptide. Such targeting groups are well known in the art and are described in detail in WO 2008/134761.

In some embodiments, the T targeting group is a moiety selected from a tumor homing group, a prostate specific membrane antigen homing peptide, an aminopeptidate N homing peptide, a Her-2 homing peptide, a colon cancer homing peptide, a VEGFR1 homing peptide, or a CXCR4 homing peptide.

In certain embodiments, the present invention provides a micelle, having SN-38 encapsulated therein, comprising a multiblock copolymer of formula VIII, as defined above and described herein.

In certain embodiments, the present invention provides a micelle, having SN-38 encapsulated therein, comprising a multiblock copolymer of formula IX:

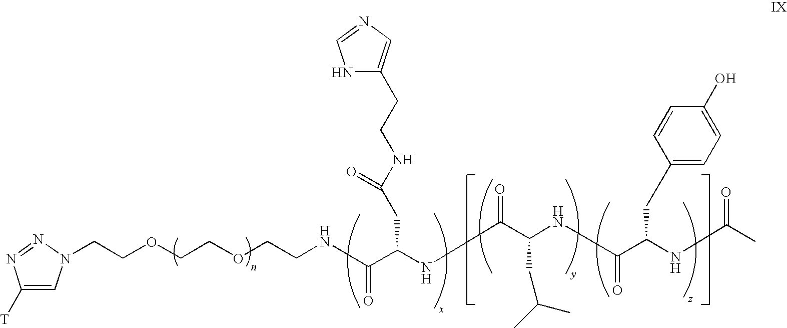

wherein:
T is a targeting group moiety;
n is 110 to 450;
x is 3 to 50;
y is 5 to 50;
z is 5 to 50.

As defined generally above, the n group of formula IX is 110-450. In certain embodiments, the present invention provides compounds of formula IX, as described above, wherein n is about 225. In other embodiments, n is about 270. In other embodiments, n is about 350. In other embodiments, n is about 110. In other embodiments, n is about 450. In other embodiments, n is selected from 110±10, 180±10, 225±10, 275±10, 315±10, or 450±10.

In certain embodiments, the x group of formula IX is about 3 to about 50. In certain embodiments, the x group of formula IX is about 10. In other embodiments, x is about 20. According to yet another embodiment, x is about 15. In other embodiments, x is about 5. In other embodiments, x is selected from 5±3, 10±3, 10±5, 15±5, or 20±5.

In certain embodiments, the y group of formula IX is about 5 to about 50. In certain embodiments, the y group of formula IX is about 10. In other embodiments, y is about 20. According to yet another embodiment, y is about 15. In other embodiments, y is about 30. In other embodiments, y is selected from 10±3, 15±3, 17±3, 20±5, 30±5, or 40±5.

In certain embodiments, the z group of formula IX is about 5 to about 50. In certain embodiments, the z group of formula IX is about 10. In other embodiments, z is about 20. According to yet another embodiment, z is about 15. In other embodiments, z is about 30. In other embodiments, z is selected from 10±3, 15±3, 17±3, 20±5, 30±5, or 40±5.

In some embodiments, the T targeting group moiety of formula IX is a moiety selected from folate, a Her-2 binding peptide, a urokinase-type plasminogen activator receptor (uPAR) antagonist, a CXCR4 chemokine receptor antagonist, a GRP78 peptide antagonist, an RGD peptide, an RGD cyclic peptide, a luteinizing hormone-releasing hormone (LHRH) antagonist peptide, an aminopeptidase targeting peptide, a brain homing peptide, a kidney homing peptide, a heart homing peptide, a gut homing peptide, an integrin homing peptide, an angiogencid tumor endothelium homing peptide, an ovary homing peptide, a uterus homing peptide, a sperm homing peptide, a microglia homing peptide, a synovium homing peptide, a urothelium homing peptide, a prostate homing peptide, a lung homing peptide, a skin homing peptide, a retina homing peptide, a pancreas homing peptide, a liver homing peptide, a lymph node homing peptide, an adrenal gland homing peptide, a thyroid homing peptide, a bladder homing peptide, a breast homing peptide, a neuroblastoma homing peptide, a lymphona homing peptide, a muscle homing peptide, a wound vasculature homing peptide, an adipose tissue homing peptide, a virus binding peptide, or a fusogenic peptide. Such targeting groups are well known in the art and are described in detail in WO 2008/134761.

In some embodiments, the T targeting group is a moiety selected from a tumor homing group, a prostate specific membrane antigen homing peptide, an aminopeptidate N homing peptide, a Her-2 homing peptide, a colon cancer homing peptide, a VEGFR1 homing peptide, or a CXCR4 homing peptide.

In certain embodiments, the present invention provides a micelle, having SN-38 encapsulated therein, comprising a multiblock copolymer of formula IX, as defined above and described herein.

In certain embodiments, the present invention provides a micelle, having SN-38 encapsulated therein, comprising a multiblock copolymer of formula X:

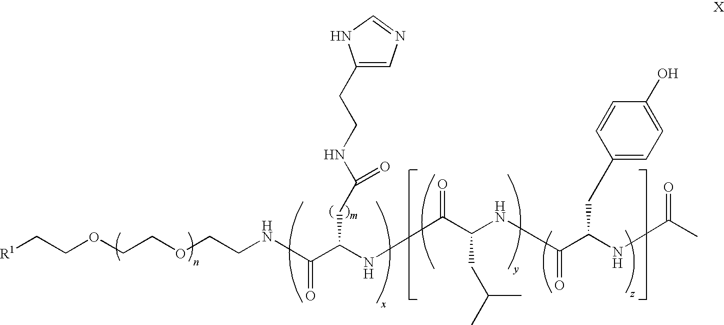

wherein:
R¹ is —OCH₃, —N₃, or

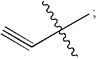

m is 1 or 2
n is 110 to 450;
x is 3 to 50;
y is 5 to 50;
z is 5 to 50.

As defined generally above, the n group of formula X is 110-450. In certain embodiments, the present invention provides compounds of formula X, as described above, wherein n is about 225. In other embodiments, n is about 270. In other embodiments, n is about 350. In other embodiments, n is about 110. In other embodiments, n is about 450. In other embodiments, n is selected from 110±10, 180±10, 225±10, 275±10, 315±10, or 450±10.

In certain embodiments, the x group of formula X is about 3 to about 50. In certain embodiments, the x group of formula X is about 10. In other embodiments, x is about 20. According to yet another embodiment, x is about 15. In other embodiments, x is about 5. In other embodiments, x is selected from 5±3, 10±3, 10±5, 15±5, or 20±5.

In certain embodiments, the y group of formula X is about 5 to about 50. In certain embodiments, the y group of formula X is about 10. In other embodiments, y is about 20. According to yet another embodiment, y is about 15. In other embodiments, y is about 30. In other embodiments, y is selected from 10±3, 15±3, 17±3, 20±5, 30±5, or 40±5.

In certain embodiments, the z group of formula X is about 5 to about 50. In certain embodiments, the z group of formula X is about 10. In other embodiments, z is about 20. According to yet another embodiment, z is about 15. In other embodiments, z is about 30. In other embodiments, z is selected from 10±3, 15±3, 17±3, 20±5, 30±5, or 40±5.

In some embodiments, the present invention provides a micelle, having an anthracycline encapsulated therein, comprising a multiblock copolymer of formula X and a multiblock copolymer of formula IX, wherein each of formula X and formula IX are as defined above and described herein, wherein the ratio of Formula X to Formula IX is between about 1000:1 and about 1:1. In other embodiments, the ratio is about 1000:1, about 100:1, about 50:1, about 33:1, about 25:1, about 20:1, about 10:1, about 5:1, or about 4:1. In yet other embodiments, the ratio is between about 100:1 and about 25:1.

### B. Crosslinked SN-38 Loaded Micelles

Crosslinking reactions designed for drug delivery preferably meet a certain set of requirements to be deemed safe and useful for in vivo applications. For example, in other embodiments, the crosslinking reaction would utilize non-cytotoxic reagents, would be insensitive to water, would not alter the drug to be delivered, and in the case of cancer therapy, would be reversible at pH levels commonly encountered in tumor tissue (pH ˜6.8) or acidic organelles in cancer cells (pH ˜5.0-6.0).

In certain embodiments, the crosslinking chemistry utilizes zinc-mediated coupling of carboxylic acids, a highly selective and pH-sensitive reaction that is performed in water. This reaction, which is widely used in cough lozenge applications, involves the association of zinc ions with carboxylic acids at basic pH. See Bakar, N. K. A.; Taylor, D. M.; Williams, D. R. Chem. Spec. Bioavail. 1999, 11, 95-101; and Eby, G. A. J. Antimicrob. Chemo. 1997, 40, 483-493. These zinc-carboxylate bonds readily dissociate in the presence of acid.

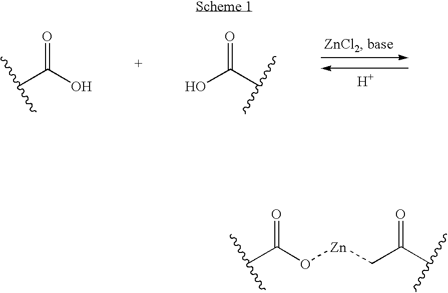

Scheme 1 above illustrates the reaction of an aqueous zinc ion (e.g. from zinc chloride) with two equivalents of an appropriate carboxylic acid to form the zinc dicarboxylate. This reaction occurs rapidly and irreversibly in a slightly basic pH environment but upon acidification, is reversible within a tunable range of pH 4.0-6.8 to reform ZnX₂, where X is the conjugate base. One of ordinary skill in the art will recognize that a variety of natural and unnatural amino acid side-chains have a carboxylic acid moiety that can be crosslinked by zinc or another suitable metal.

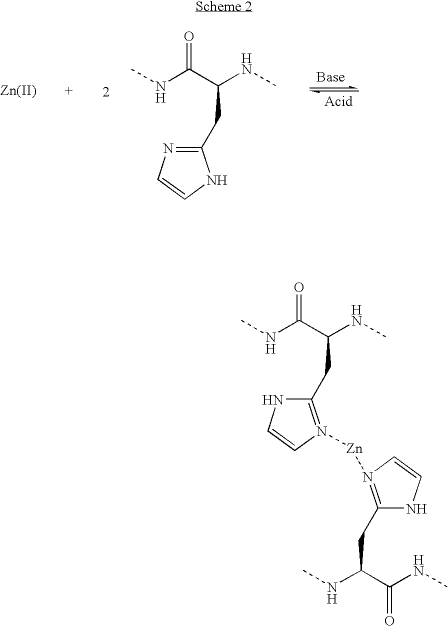

Scheme 2 above illustrates the reaction of an aqueous zinc (II) ion (e.g. from zinc chloride or zinc acetate) with two equivalents of an appropriate imidazole (e.g. histidine) to form a zinc-histidine complex. This reaction occurs rapidly in a slightly basic pH environment and is reversible upon acidification to pH less than 6. (Tezcan, et. al. J. Am. Chem. Soc. 2007, 129, 13347-13375.)

In certain embodiments, R^{x} is a histidine side-chain crosslinked with zinc. Without wishing to be bound by any particular theory, it is believed that zinc-histidine crosslinks are stable in the blood compartment (pH 7.4), allowing for effective accumulation of therapeutic loaded micelles in solid tumors by passive and/or active targeting mechanisms. In the presence of lactic acid concentrations commonly encountered in solid tumors or hydrochloric acid in acidic organelles of cancer cells, rapid degradation of the metal crosslinks occurs which leads to micelle dissociation and release of SN-38 at the tumor site.

The choice of zinc as a crosslinking metal is advantageous for effective micelle crosslinking Zinc chloride and the zinc lactate by-product are generally recognized as non-toxic, and other safety concerns are not anticipated. Pharmaceutical grade zinc chloride is commonly used in mouthwash and as a chlorophyll stabilizer in vegetables while zinc lactate is used as an additive in toothpaste and drug preparation. While zinc has been chosen as an exemplary metal for micelle crosslinking, it should be noted that many other metals undergo acid sensitive coupling with imidazole derivatives. These metals include calcium, iron, copper, nickel and other transition metals. One or more of these metals can be substituted for zinc.

The ultimate goal of metal-mediated crosslinking is to ensure micelle stability when diluted in the blood (pH 7.4) followed by rapid dissolution and polynucleotide release in response to a finite pH change such as those found in tumor environments or in intracellular compartments. Previous reports suggest that the zinc-histidine bonds are stable above a threshold pH, below which dissociation to zinc ions and histidine occurs. (Tezcan, et. al. J. Am. Chem. Soc. 2007, 129, 13347-13375.)

In certain embodiments, R^{x} is a imidazole-containing side-chain group crosslinked with nickel. Without wishing to be bound to any particular theory, it is believed that the nickel will interact with the imidazole moiety in a pH dependent fashion.

In certain embodiments, SN-38 loaded micelles of the present invention comprise a crosslinked multiblock polymer of formula XI:

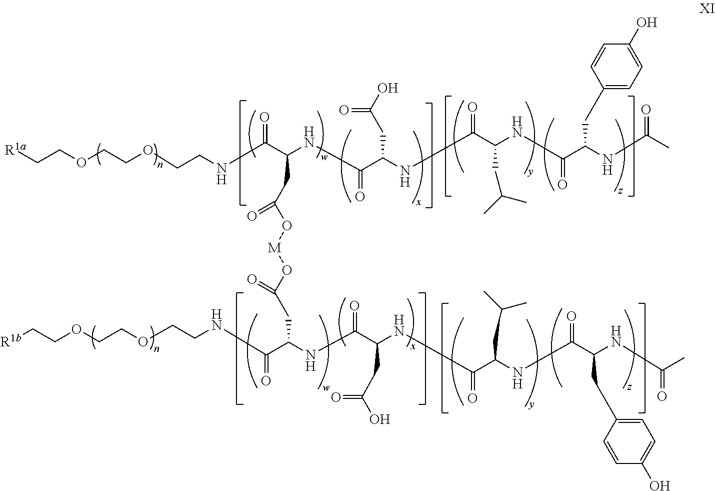

wherein:
R^{1a} and R^{1b} are independently selected from —OCH₃, —N₃,

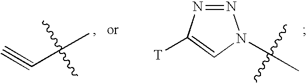

T is a targeting group moiety;
M is a suitable metal ion;
n is 110 to 450;
w is 3 to 50;
x is 0 to 50, provided that the sum of w and x is no more than 50;
y is 5 to 50; and
z is 5 to 50.

As defined generally above, the n group of formula XI is 110-450. In certain embodiments, the present invention provides compounds of formula XI, as described above, wherein n is about 225. In other embodiments, n is about 270. In other embodiments, n is about 350. In other embodiments, n is about 110. In other embodiments, n is about 450. In other embodiments, n is selected from 110±10, 180±10, 225±10, 275±10, 315±10, or 450±10.

In certain embodiments, the w group of formula XI is about 3 to about 50. In certain embodiments, the w group of formula XI is 10. In other embodiments, w is about 5-10. According to yet another embodiment, w is about 1-10. In other embodiments, w is about 5. In other embodiments, w is selected from 5±3, 10±3, 10±5, 15±5, or 20±5.

In certain embodiments, the x group of formula XI is about 0 to about 50. In certain embodiments, the x group of formula XI is 0. In other embodiments, x is about 0-5. According to yet another embodiment, x is about 10. In other embodiments, x is about 5. In other embodiments, x is selected from 3±3, 5±3, 10±5, 15±5, or 20±5.

In certain embodiments, the y group of formula XI is about 5 to about 50. In certain embodiments, the y group of formula XI is about 10. In other embodiments, y is about 20. According to yet another embodiment, y is about 15. In other embodiments, y is about 30. In other embodiments, y is selected from 10±3, 15±3, 17±3, 20±5, 30±5, or 40±5.

In certain embodiments, the z group of formula XI is about 5 to about 50. In certain embodiments, the z group of formula XI is about 10. In other embodiments, z is about 20. According to yet another embodiment, z is about 15. In other embodiments, z is about 30. In other embodiments, z is selected from 10±3, 15±3, 17±3, 20±5, 30±5, or 40±5.

In some embodiments, the T targeting group moiety of formula XI is a moiety selected from folate, a Her-2 binding peptide, a urokinase-type plasminogen activator receptor (uPAR) antagonist, a CXCR4 chemokine receptor antagonist, a GRP78 peptide antagonist, an RGD peptide, an RGD cyclic peptide, a luteinizing hormone-releasing hormone (LHRH) antagonist peptide, an aminopeptidase targeting peptide, a brain homing peptide, a kidney homing peptide, a heart homing peptide, a gut homing peptide, an integrin homing peptide, an angiogencid tumor endothelium homing peptide, an ovary homing peptide, a uterus homing peptide, a sperm homing peptide, a microglia homing peptide, a synovium homing peptide, a urothelium homing peptide, a prostate homing peptide, a lung homing peptide, a skin homing peptide, a retina homing peptide, a pancreas homing peptide, a liver homing peptide, a lymph node homing peptide, an adrenal gland homing peptide, a thyroid homing peptide, a bladder homing peptide, a breast homing peptide, a neuroblastoma homing peptide, a lymphona homing peptide, a muscle homing peptide, a wound vasculature homing peptide, an adipose tissue homing peptide, a virus binding peptide, or a fusogenic peptide. Such targeting groups are well known in the art and are described in detail in WO 2008/134761.

In some embodiments, the T targeting group is a moiety selected from a tumor homing group, a prostate specific membrane antigen homing peptide, an aminopeptidate N homing peptide, a Her-2 homing peptide, a colon cancer homing peptide, a VEGFR1 homing peptide, or a CXCR4 homing peptide.

In certain embodiments, the -M- moiety of formula XI is zinc. In other embodiments, M is selected from Ag, Fe, Cu, Ca, Mg, Ni, or Co. One of ordinary skill in the art will recognize that an SN-38 loaded micelle of formula X can be prepared from a mixture of Formula I and one or more polymers selected from formula II, III, IV, or V.

In certain embodiments, SN-38 loaded micelles of the present invention comprise a crosslinked multiblock polymer of formula XII:

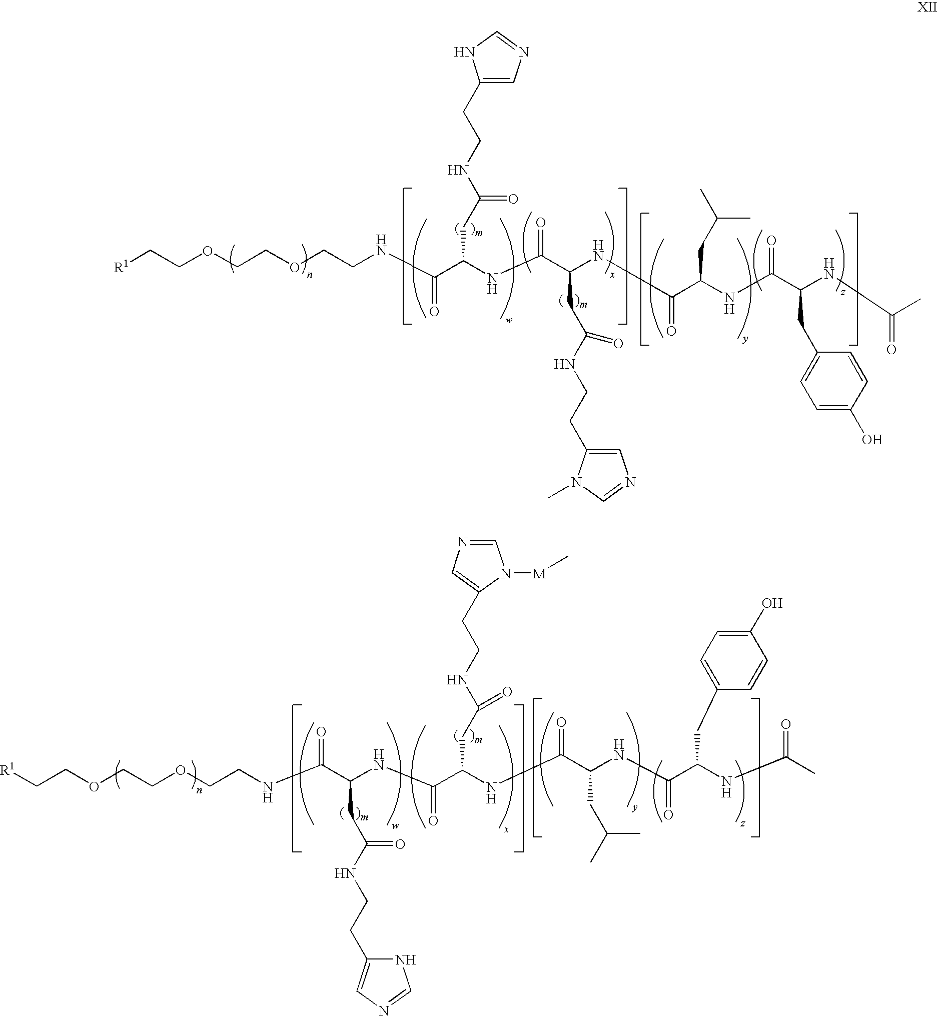

wherein:
each n is independently 110 to 450;
each m is independently 1 or 2;
each w is independently 0-20;
each x is independently 1-20;
each y is independently 5 to 50;
each z is independently 5 to 50.
M is Zn, Fe, Co, or Ni; and
each R¹ is independently —N₃, —OCH₃ or

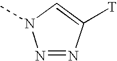

wherein T is a targeting group moiety.

As defined generally above, each n group of formula XII is independently 110-450. In certain embodiments, the present invention provides compounds of formula XII, as described above, wherein n is about 225. In other embodiments, n is about 270. In other embodiments, n is about 350. In other embodiments, n is about 110. In other embodiments, n is about 450. In other embodiments, n is selected from 110±10, 180±10, 225±10, 275±10, 315±10, or 450±10.

In certain embodiments, each x group of formula XII is independently about 1 to about 30. In certain embodiments, the x group of formula X is about 10. In other embodiments, x is about 20. According to yet another embodiment, x is about 15. In other embodiments, x is about 5. In other embodiments, x is selected from 3±2, 5±3, 10±3, 10±5, 15±5, or 20±5.

In certain embodiments, each y group of formula XII is independently about 5 to about 50. In certain embodiments, the y group of formula XII is about 10. In other embodiments, y is about 20. According to yet another embodiment, y is about 15. In other embodiments, y is about 30. In other embodiments, y is selected from 10±3, 15±3, 17±3, 20±5, 30±5, or 40±5.

In certain embodiments, each z group of formula XII is independently about 5 to about 50. In certain embodiments, the z group of formula XII is about 10. In other embodiments, z is about 20. According to yet another embodiment, z is about 15. In other embodiments, z is about 30. In other embodiments, z is selected from 10±3, 15±3, 17±3, 20±5, 30±5, or 40±5.

In some embodiments, each T targeting group moiety of formula XII is independently a moiety selected from folate, a Her-2 binding peptide, a urokinase-type plasminogen activator receptor (uPAR) antagonist, a CXCR4 chemokine receptor antagonist, a GRP78 peptide antagonist, an RGD peptide, an RGD cyclic peptide, a luteinizing hormone-releasing hormone (LHRH) antagonist peptide, an aminopeptidase targeting peptide, a brain homing peptide, a kidney homing peptide, a heart homing peptide, a gut homing peptide, an integrin homing peptide, an angiogencid tumor endothelium homing peptide, an ovary homing peptide, a uterus homing peptide, a sperm homing peptide, a microglia homing peptide, a synovium homing peptide, a urothelium homing peptide, a prostate homing peptide, a lung homing peptide, a skin homing peptide, a retina homing peptide, a pancreas homing peptide, a liver homing peptide, a lymph node homing peptide, an adrenal gland homing peptide, a thyroid homing peptide, a bladder homing peptide, a breast homing peptide, a neuroblastoma homing peptide, a lymphona homing peptide, a muscle homing peptide, a wound vasculature homing peptide, an adipose tissue homing peptide, a virus binding peptide, or a fusogenic peptide. Such targeting groups are well known in the art and are described in detail in WO 2008/134761.

In some embodiments, the T targeting group is a moiety selected from a tumor homing group, a prostate specific membrane antigen homing peptide, an aminopeptidate N homing peptide, a Her-2 homing peptide, a breast cancer homing peptide, a VEGFR1 homing peptide, or a CXCR4 homing peptide.

### 4. General Methods for Providing Compounds of the Present Invention

Bifunctional PEG's are prepared according to U.S. Patent Application Publication Numbers 2006/0240092, 2006/0172914, 2006/0142506, and 2008/0035243, and Published PCT Applications WO07/127473, WO07/127440, and WO06/86325, the entirety of each of which is hereby incorporated by reference.

Multiblock copolymers of the present invention are prepared by methods known to one of ordinary skill in the art and those described in detail in U.S. patent application Ser. No. 11/325,020 filed Jan. 4, 2006 and published as US 20060172914 on Aug. 3, 2006, the entirety of which is hereby incorporated herein by reference. Generally, such multiblock copolymers are prepared by sequentially polymerizing one or more cyclic amino acid monomers onto a hydrophilic polymer having a terminal amine salt wherein said polymerization is initiated by said amine salt. In certain embodiments, said polymerization occurs by ring-opening polymerization of the cyclic amino acid monomers. In other embodiments, the cyclic amino acid monomer is an amino acid NCA, lactam, or imide. Details of preparing exemplary multiblock copolymers of the present invention are set forth in the Exemplification, infra.

Methods of preparing micelles are known to one of ordinary skill in the art. Micelles can be prepared by a number of different dissolution methods. In the direct dissolution method, the block copolymer is added directly to an aqueous medium with or without heating and micelles are spontaneously formed up dissolution. The dialysis method is often used when micelles are formed from poorly aqueous soluble copolymers. The copolymer is dissolved in a water miscible organic solvent such as N-methyl pyrrolidinone, dimethylformamide, dimethylsulfoxide, tetrahydrofuran, or dimethylacetamide, and this solution is then dialyzed against water or another aqueous medium. During dialysis, micelle formation is induced and the organic solvent is removed. Alternatively, the block copolymer can be dissolved in in a water miscible organic solvent such as N-methyl pyrrolidinone, dimethylformamide, dimethylsulfoxide, tetrahydrofuran, or dimethylacetamide and added dropwise to water or another aqueous medium. The micelles can then be isolated by filtration or lyophilization.

Emulsification methods can also be employed for micelle formation. For example, the block copolymer is dissolved in a water-immiscible, volatile solvent (e.g. dichloromethane) and added to water with vigorous agitation. As the solvent is removed by evaporation, micelles spontaneously form. Prepared micelles can then be filtered and isolated by lyophilization.

Micelles can be prepared by a number of different dissolution methods. In the direct dissolution method, the block copolymer is added directly to an aqueous medium, with or without heating, and micelles are spontaneously formed up dissolution. The dialysis method is often used when micelles are formed from poorly aqueous soluble copolymers. The copolymer is dissolved in a water miscible organic solvent such as N-methyl pyrrolidinone, dimethylformamide, dimethylsulfoxide, tetrahydrofuran, or dimethylacetamide, and this solution is then dialyzed against water or another aqueous medium. During dialysis, micelle formation is induced and the organic solvent is removed. Alternatively, the block copolymer can be dissolved in in a water miscible organic solvent such as N-methyl pyrrolidinone, dimethylformamide, dimethylsulfoxide, tetrahydrofuran, or dimethylacetamide and added dropwise to water or another aqueous medium. The micelles can then be isolated by filtration or lyophilization.

Many traditional encapsulation methods failed to allow for effective encapsulation of SN-38. We have suprisingly found that high shear environments, controlled solvent evaporation, and selection of the appropriate polymer result in successful encapsulation of SN-38. Examples of high shear environments include high shear mixers (e.g. Silverson Mixers), sonication, microfluidization, and high pressures (from about 5,000 psi to about 30,000 psi). Such methods are described in detail in the Exemplification, infra.

In one embodiment, drug-loaded micelles possessing carboxylic acid functionality in the outer core are crosslinked by addition of zinc chloride to the micelle solution along with a small amount of sodium hydroxide to neutralize any hydrochloric acid by-product. In this basic pH environment, the reaction of zinc chloride with the poly(aspartic acid) crosslinking block should be rapid and irreversible.

In another embodiment, drug loaded micelles possessing amine functionality in the outer core are crosslinked by the addition of a bifunctional, or multi-functional aldehyde-containing molecule which forms pH-reversible imine crosslinks. In another embodiment, drug loaded micelles possessing aldehyde functionality in the outer core are crosslinked by the addition of a bifunctional, or multi-functional amine-containing molecule which forms pH-reversible imine crosslinks

In another embodiment, drug loaded micelles possessing alcohol or amine functionality in the outer core are crosslinked by the addition of a bifunctional, or multi-functional carboxylic acid-containing molecules and a coupling agent to form amide or ester crosslinks. In yet another embodiment, drug loaded micelles possessing carboxylic acid functionality in the outer core are crosslinked by the addition of a bifunctional, or multi-functional amine or alcohol-containing molecules and a coupling agent to form amide or ester crosslinks. Such coupling agents include, but are not limited to, carbodiimides (e.g. 1-ethyl-3-(3-dimethylaminopropyl)-carbodiimide (EDC), diisopropyl carbodiimide (DIC), dicyclohexyl carbodiimide (DCC)), aluminium or phosphonium derivatives (e.g. PyBOP, PyAOP, TBTU, HATU, HBTU), or a combination of 1-hydroxybenzotriazole (HOBt) and a aminium or phosphonium derivative.

In another embodiment, drug loaded micelles possessing aldehyde or ketone functionality in the outer core are crosslinked by the addition of a bifunctional, or multifunctional hydrazine or hydrazide-containing molecule to form pH-reversible hydrazone crosslinks. In still other embodiments, drug loaded micelles hydrazine or hydrazide-functionality in the outer core are crosslinked by the addition of a bifunctional, or multifunctional aldehyde or ketone-containing molecule to form pH-reversible hydrazone crosslinks.

In another embodiment, drug loaded micelles possessing thiol functionality in the outer core are crosslinked by the addition of an oxidizing agent (e.g. metal oxides, halogens, oxygen, peroxides, ozone, peroxyacids, etc.) to form disulfide crosslinks It will be appreciated that disulfide crosslinks are reversible in the presence of a suitable reducing agent (e.g. glutathione, dithiothreitol (DTT), etc.).

In yet another embodiment, drug loaded micelles possessing both carboxylic acid and thiol functionality in the outer core can be dual crosslinked by the addition of an oxidizing agent (e.g. metal oxides, halogens, oxygen, peroxides, ozone, peroxyacids, etc.) to form disulfide crosslinks followed by the addition of zinc chloride to the micelle solution along with a small amount of sodium bicarbonate to neutralize any hydrochloric acid by-product. It will be appreciated that such a dual-crosslinked micelle is reversible only in the presence of acid and a reducing agent (e.g. glutathione, dithiothreitol (DTT), etc.).

### 5. Uses, Methods, and Compositions

Compositions

As described herein, micelles of the present invention having SN-38 encapsulated therein are useful for treating cancer. According to one embodiment, the present invention relates to the treatment of colorectal cancer. In another embodiment, the present invention relates to the treatment of pancreatic cancer. According to another embodiment, the present invention relates to a method of treating breast cancer. In another embodiment, the present invention relates to the treatment of prostate cancer. According to another embodiment, the present invention relates to a method of treating a cancer selected from ovary, cervix, testis, genitourinary tract, esophagus, larynx, glioblastoma, neuroblastoma, stomach, skin, keratoacanthoma, lung, epidermoid carcinoma, large cell carcinoma, small cell carcinoma, lung adenocarcinoma, bone, colon, adenoma, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, Hodgkin's, hairy cells, buccal cavity and pharynx (oral), lip, tongue, mouth, pharynx, small intestine, large intestine, rectum, brain and central nervous system, and leukemia, comprising administering a micelle in accordance with the present invention having SN-38 encapsulated therein.

P-glycoprotein (Pgp, also called multidrug resistance protein) is found in the plasma membrane of higher eukaryotes where it is responsible for ATP hydrolysis-driven export of hydrophobic molecules. In animals, Pgp plays an important role in excretion of and protection from environmental toxins; when expressed in the plasma membrane of cancer cells, it can lead to failure of chemotherapy by preventing the hydrophobic chemotherapeutic drugs from reaching their targets inside cells. Indeed, Pgp is known to transport hydrophobic chemotherapeutic drugs out of tumor cells. According to one aspect, the present invention provides a method for delivering a SN-38 to a cancer cell while preventing, or lessening, Pgp excretion of that chemotherapeutic drug, comprising administering a drug-loaded micelle comprising a multiblock polymer of the present invention loaded with SN-38.

Compositions

According to another embodiment, the invention provides a composition comprising a micelle of this invention or a pharmaceutically acceptable derivative thereof and a pharmaceutically acceptable carrier, adjuvant, or vehicle. In certain embodiments, the composition of this invention is formulated for administration to a patient in need of such composition. In other embodiments, the composition of this invention is formulated for oral administration to a patient.

The term “patient”, as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts.

Salts derived from appropriate bases include alkali metal (e.g., sodium and potassium), alkaline earth metal (e.g., magnesium), ammonium and N⁺(C₁₋₄ alkyl)₄ salts. This invention also envisions the quaternization of any basic nitrogen-containing groups of the compounds disclosed herein. Water or oil-soluble or dispersible products may be obtained by such quaternization.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. In certain embodiments, pharmaceutically acceptable compositions of the present invention are enterically coated.

Alternatively, the pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

The pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutically acceptable compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

The pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In certain embodiments, the pharmaceutically acceptable compositions of this invention are formulated for oral administration.

The amount of the compounds of the present invention that may be combined with the carrier materials to produce a composition in a single dosage form will vary depending upon the host treated, the particular mode of administration. Preferably, the compositions should be formulated so that a dosage of between 0.01-100 mg/kg body weight/day of the drug can be administered to a patient receiving these compositions.

It will be appreciated that dosages typically employed for the encapsulated drug are contemplated by the present invention. In certain embodiments, a patient is administered a drug-loaded micelle of the present invention wherein the dosage of the drug is equivalent to what is typically administered for that drug. In other embodiments, a patient is administered a drug-loaded micelle of the present invention wherein the dosage of the drug is lower than is typically administered for that drug.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

In order that the invention described herein may be more fully understood, the following examples are set forth. It will be understood that these examples are for illustrative purposes only and are not to be construed as limiting this invention in any manner.

### Exemplification

### Preparation of Bifunctional PEGs and Multiblock Copolymers of the Present Invention

As described generally above, multiblock copolymers of the present invention are prepared using the heterobifunctional PEGs described herein and in U.S. patent application Ser. No. 11/256,735, filed Oct. 24, 2005, published as WO2006/047419 on May 4, 2006 and published as US 20060142506 on Jun. 29, 2006, the entirety of which is hereby incorporated herein by reference. The preparation of multiblock polymers in accordance with the present invention is accomplished by methods known in the art, including those described in detail in U.S. patent application Ser. No. 11/325,020, filed Jan. 4, 2006, published as WO2006/74202 on Jul. 13, 2006 and published as US 20060172914 on Aug. 3, 2006, the entirety of which is hereby incorporated herein by reference.

In each of the Examples below, where an amino acid, or corresponding NCA, is designated “D”, then that amino acid, or corresponding NCA, is of the D-configuration. Where no such designation is recited, then that amino acid, or corresponding NCA, is of the L-configuration.

SN-38 loading was determined by weighing ca. 10-20 mg of drug loaded micelle into a 10 mL volumetric flask and filling to volume with 2 mL of DMSO and 8 mL of acetonitrile. 10 μL of this solution was injected onto a Waters 2695 HPLC with a 996 photodiode array detector and ES Industries Chromegabond Alkyl-Phenyl column (300mm) eluting with 50% 25 mM monobasic sodium phosphate buffer (pH ˜3.1) and 50% acetonitrile at 1 mL/min. SN-38 eluted at 4.0 minutes under these conditions. Quantitation was performed from a calibration curve constructed from known concentrations of SN-38 standard injections from chromatograms extracted at 265 nm. Area under the curve (AUC) can be converted to concentration with the following equation:

$$\frac{μ g}{10 μ L} = \frac{AUC}{3936855} = \frac{mg}{10 mL}$$

Particle size distribution was determined by dynamic light scattering. Lyopholyzed polymers were dissolved at 5 mg/mL in phosphate buffered saline at pH 7.4 and equilibrated overnight. Each sample was analyzed in a PSS NICOMP 380 with a 690 nm laser at a 90 degree angle or in a Wyatt Dynapro with a 658 nm laser. DLS sizing data was recorded from the volume weighted Gaussian distribution (Nicomp) or Regularization fit (DynaPro).

### Example 1

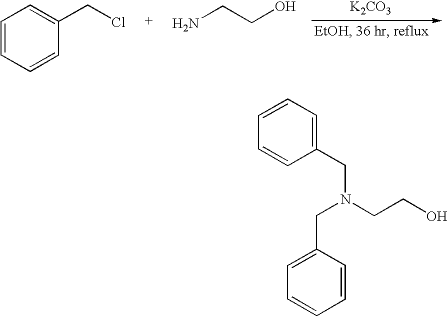

Dibenzylamino Ethanol Benzyl chloride (278.5 g, 2.2 mol), ethanol amine (60 mL, 1 mol), potassium carbonate (283.1 g, 2.05 mol) and ethanol (2 L) were mixed together in a 3 L 3-neck flask, fitted with an overhead stirrer, a condenser and a glass plug. The apparatus was heated up to reflux for 36 hr, after which the insoluble solid was filtered through a medium frit. The filtrate was recovered and ethanol was removed by rotary evaporation. The viscous liquid was redissolved in ether, the solid suspension removed by filtration and extracted twice against water. The ether solution was kept and the aqueous layer was extracted twice with dichloromethane (2×400 mL). The fraction were recombined, dried over MgSO₄, stirred over carbon black for 15 min and filtered through a celite pad. Dichloromethane was removed and the solid was redissolved into a minimal amount of ether (combined volume of 300 mL with the first ether fraction, 300 mL). Hexanes (1700 mL) was added and the solution was heated up gently till complete dissolution of the product. The solution was then cooled down gently, placed in the fridge (+4° C.) overnight and white crystals were obtained. The recrystallization was done a second time. 166.63 g, 69% yield. ¹H NMR (d₆-DMSO) δ 7.39-7.24 (10H), 4.42 (1H), 3.60 (4H), 3.52 (2H), 2.52 (2H).

### Example 2

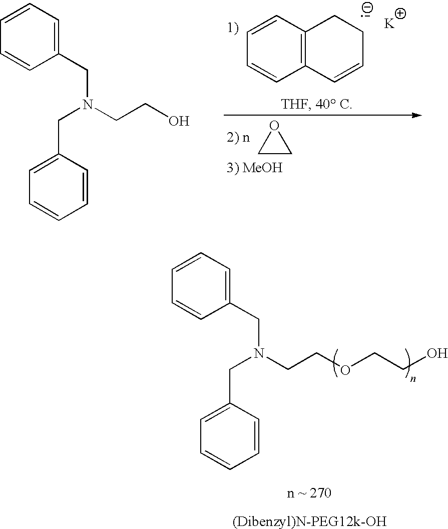

(Dibenzyl)-N-Poly(ethylene oxide)₂₇₀-OH Potassium is freshly cut under dry hexanes to remove all oxide. Potassium (3.13 g, 80 mmol) is weighed in a tared vial containing dry hexanes, then transferred with tweezers to a Schlenk flask with an Argon purge. The flask is then evacuated and any residual hexanes is allowed to evaporate, then the flask backfilled with Argon. Separately, recrystallized, sublimed naphthalene (12.30 g, 100 mmol) is added to a 250 mL round bottom flask. The flask and its contents are dried under vacuum for 15 minutes, then backfilled with Argon. Dry THF (200 mL) is then added to the Schlenk flask containing the potassium, and dry THF (200 mL) is added to the flask containing the naphthalene. Once the naphthalene is completely dissolved in the THF, the entire solution is transferred to the Schlenk flask. A green color begins to appear within 1 minute of the naphthalene solution addition. The solution is stirred overnight to allow for complete reaction, yielding ˜400 mL of a 0.2 M potassium naphtalenide solution. The solution is used within 48 hours of preparation. Any unused solution is quenched by the addition of isopropyl alcohol.

The glassware was assembled while still warm. Vacuum was then applied to the assembly and the ethylene oxide line to about 10 mTorr. The setup was backfilled with argon. 2-Dibenzylamino ethanol from Example 1 (3.741 g, 40.4 mmol) was introduced via the sidearm of the jacketed flask under argon overpressure. Two vacuum/argon backfill cycles were applied to the whole setup. THF line was connected to the 14/20 side-arm and vacuum was applied to the whole setup. At this stage, the addition funnel was closed and left under vacuum. THF (4 L) was introduced via the side-arm in the round bottom flask under an argon overpressure. An aliquot of the THF added to the reaction vessel was collected and analyzed by Karl-Fisher colorometric titration to ensure water content of the THF is less than 6 ppm. Next, 2-dibenzylamino ethanol was converted to potassium 2-dibenzylamino ethoxide via addition of potassium naphthalenide (200 mL). Ethylene oxide (500 ml, 10.44 mol) was condensed under vacuum at −30° C. into the jacketed addition funnel, while the alkoxide solution was cooled to 10° C. Once the appropriate amount of ethylene oxide was condensed, the flow of ethylene oxide was stopped, and the liquid ethylene oxide added directly to the cooled alkoxide solution. After complete ethylene oxide addition, the addition funnel was closed and the reaction flask backfilled with argon. While stirring, the following temperature ramp was applied to the reaction: 12 hrs at 20° C., 1 hr from 20° C. to 40° C. and 3 days at 40° C. The reaction went from a light green tint to a golden yellow color. Upon termination with an excess methanol, the solution color changed to light green. The solution was precipitated into ether and isolated by filtration. 459 g, 99% yield was recovered after drying in a vacuum oven overnight. ¹H NMR (d6-DMSO) δ 7.4-7.2 (10H), 4.55 (1H), 3.83-3.21 (910 H) ppm.

### Example 3

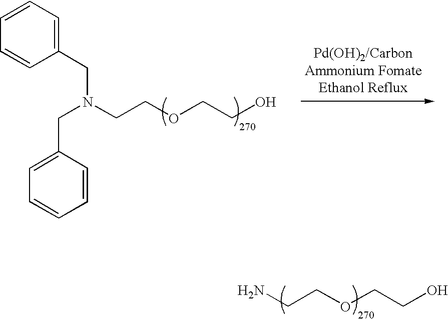

NH₂-Poly(ethylene oxide)₂₇₀-OH (Dibenzyl)-N-poly(ethylene oxide)₂₇₀-OH from Example 2 (455 g, 39.56 mmol) was split into two equal amounts and was introduced into two 2L flasks. A separate batch of (dibenzyl)-N-poly(ethylene oxide)₂₇₀-OH (273 g, 23.74 mmol) was added to a third 2 L flask. The following steps were repeated for each flask. The following steps were repeated for each flask. (Dibenzyl)-N-poly(ethylene oxide)₂₇₀-OH (˜225 g), Pd(OH)₂/C (32 g, 45.6 mmol), ammonium formate (80 g, 1.27 mol) and ethanol (1.2 L) were mixed together in a 2 L flask. The reaction was heated to 80° C. while stirring for 24 hrs. The reaction was cooled to room temperature and filtered through a triple layer Celite/MgSO₄/Celite pad. The MgSO₄ powder is fine enough that very little Pd(OH)₂/C permeates through the pad. Celite helps prevent the MgSO₄ layer from cracking At this stage, the three filtrates were combined, precipitated into ˜30 L of ether and filtered through a medium glass frit. The wet polymer was then dissolved into 4 L of water, 1 L of brine and 400 mL of saturated K₂CO₃ solution. The pH was checked to be ˜11 by pH paper. The aqueous solution was introduced into a 12 L extraction funnel, rinsed once with 4 L of ether and extracted 4 times with dichloromethane (6 L, 6 L, 6 L, 2 L). Dichloromethane fractions were recombined, dried over MgSO₄ (3 kg) filtered, concentrated to ˜3 L by rotary evaporation and precipitated into diethyl ether (30 L). 555 g, 75% yield of the title compound was recovered after filtration and evaporation to dryness in a vacuum oven. ¹H NMR (d6-DMSO) 4.55 (1H), 3.83-3.21 (910 H), 2.96 (2H) ppm.

### Example 4

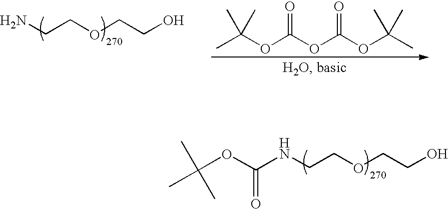

Boc-NH-Poly(ethylene oxide)₂₇₀-OH NH₂-Poly(ethylene oxide)₂₇₀-OH (555 g, 48.26 mmol) from Example 3 was dissolved into 4 L of DI water. A saturated solution of K₂CO₃ (120 mL) was added, to keep the pH basic (pH ˜11 with pH paper). Di-tert-butyl dicarbonate (105 g, 0.48 mol) was added to the aqueous solution of NH₂-poly(ethylene oxide)₂₇₀-OH and allowed to stir at room temperature overnight. At this stage, a 5 mL aliquot of the reaction was extracted with 10 mL of dichloromethane and the dichloromethane extract precipitated into ether. A ¹H NMR was run to ensure completion of the reaction. Thereafter, the aqueous solution was placed into a 12 L extraction funnel, was rinsed once with ether (4 L) and extracted three times with dichloromethane (6 L, 6 L and 6 L). The organic fractions were recombined, dried over MgSO₄ (3 kg), filtered, concentrated to ˜4 L and precipitated into 30 L of ether. The white powder was filtered and dried overnight in a vacuum oven, giving 539 g of the title compound in 97% yield. ¹H NMR (d₆-DMSO) δ 6.75 (1H), 4.55 (1H), 3.83-3.21 (910 H), 3.06 (2H), 1.37 (9H) ppm.

### Example 5

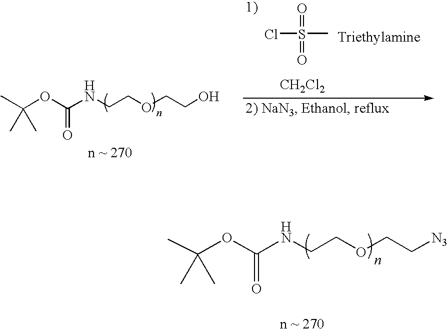

Boc-NH-Poly(ethylene oxide)₂₇₀-N₃ Boc-NH-Poly(ethylene oxide)₂₇₀-OH (539 g, 49.9 mmol) from Example 4 was placed into a 6 L jacketed flask and dried by azeotropic distillation from toluene (3 L). It was then dissolved into 3 L of dry dichloromethane under inert atmosphere. The solution was cooled to 0° C., methanesulfonyl chloride (10.9 mL, 140.8 mmol) was added followed by triethylamine (13.1 mL, 94 mmol). The reaction was allowed to warm to room temperature and proceeded overnight under inert atmosphere. The solution was evaporated to dryness by rotary evaporation and used as-is for the next step.

NaN₃ (30.5 g, 470 mmol) and 3 L of ethanol were added to the flask containing the polymer. The solution was heated to 80° C. and allowed to react overnight. It was then evaporated to dryness by rotary evaporation (bath temperature of 55° C.) and dissolved in 2 L of dichloromethane. The latter solution was the filtered through a Buchner funnel fitted with a Whatman paper #1 to remove most of the salts. The solution was concentrated down to ˜1 L by rotary evaporation. The product was purified by silica gel flash column chromatography using a 8 in. diameter column with a coarse frit. About 7 L of dry silica gel were used. The column was packed with 1:99 MeOH/CH₂Cl₂ and the product was loaded and eluted onto the column by pulling vacuum from the bottom of the column. The elution profile was the following: 1:99 MeOH/CH₂Cl₂ for 1 column volume (CV), 3:97 MeOH/CH₂Cl₂ for 2 CV and 10:90 MeOH/CH₂Cl₂ for 6 CV. The different polymer-containing fractions were recombined (˜40 L of dichloromethane), concentrated by rotary evaporation and precipitated into a 10-fold excess of diethyl ether. The title compound was recovered by filtration as a white powder and dried overnight in vacuo, giving 446.4 g, 82% yield. ¹H NMR (d₆-DMSO) δ 6.75 (1H), 3.83-3.21 (910 H), 3.06 (2H), 1.37 (9H) ppm. Mₙ (MALDI-TOF)=11,554 g/mol. PDI (DMF GPC)=1.04

### Example 6

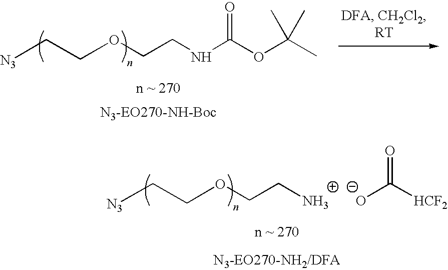

DFA^{+ -}NH₃-Poly(ethylene oxide)₂₇₀-N₃ Boc-NH-Poly(ethylene oxide)₂₇₀-N₃ (313 g, 27.2 mmol) from Example 5 was weighed into a 2 L beaker, 600 mL of DFA, 600 mL of dichloromethane were added. The solution was stirred at room temperature for 32 hr and the polymer was recovered by two consecutive precipitation in ether (2×30 L). The white powder was dried overnight in a vacuum oven to afford the title compound. (306 g, 98% yield). ¹H NMR (d₆-DMSO) δ 7.67 (3H), 6.13 (1H), 3.82-3.00 (1060H), 2.99 (2H).

### Example 7

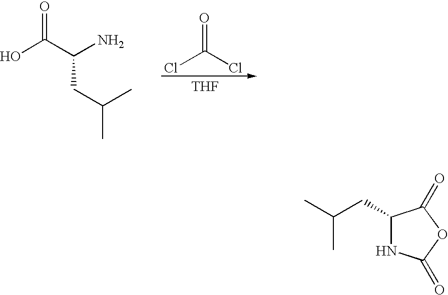

D-Leucine NCA H-D-Leu-OH (100 g, 0.76 mol) was suspended in 1 L of anhydrous THF and heated to 50° C. while stirring heavily. Phosgene (20% in toluene) (500 mL, 1 mol) was added the amino acid suspension. After 1 h 20 min, the amino acid dissolved, forming a clear solution. The solution was concentrated on the rotovap, transferred to a beaker, and hexane was added to precipitate the product. The white solid was isolated by filtration and dissolved in toluene (˜700 mL) with a small amount of THF (˜60 mL). The solution was filtered over a bed of Celite to remove any insoluble material. An excess of hexane (˜4 L) was added to the filtrate to precipitate the product. The NCA was isolated by filtration and dried in vacuo. (91 g, 79% yield) D-Leu NCA was isolated as a white, crystalline solid. ¹H NMR (d₆-DMSO) δ 9.13 (¹H), 4.44 (1H), 1.74 (1H), 1.55 (2H), 0.90 (6H) ppm.

### Example 8

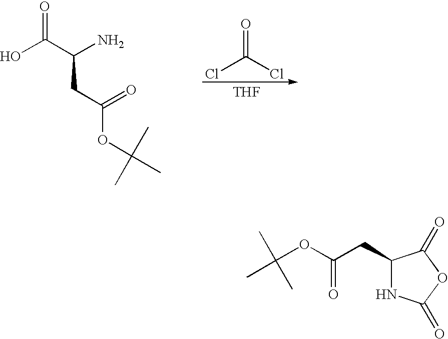

tert-Butyl Aspartate NCA H-Asp(OBu)-OH (120 g, 0.63 mol) was suspended in 1.2 L of anhydrous THF and heated to 50° C. while stirring heavily. Phosgene (20% in toluene) (500 mL, 1 mol) was added the amino acid suspension. After 1 h 30 min, the amino acid dissolved, forming a clear solution. The solution was concentrated on the rotovap, transferred to a beaker, and hexane was added to precipitate the product. The white solid was isolated by filtration and dissolved in anhydrous THF. The solution was filtered over a bed of Celite to remove any insoluble material. An excess of hexane was added to precipitate the product. The NCA was isolated by filtration and dried in vacuo. 93 g (68%) of Asp(OBu) NCA was isolated as a white, crystalline solid. ¹H NMR (d₆-DMSO) δ 8.99 (1H), 4.61 (1H), 2.93 (1H), 2.69 (1H), 1.38 (9H) ppm.

### Example 9

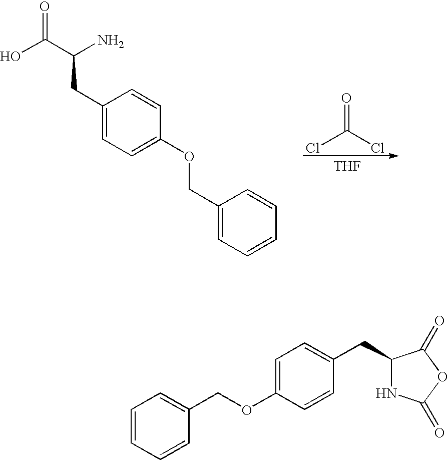

Benzyl Tyrosine NCA H-Tyr(OBzl)-OH (140 g, 0.52 mol) was suspended in 1.5 L of anhydrous THF and heated to 50° C. while stirring heavily. Phosgene (20% in toluene) (500 mL, 1 mol) was added the amino acid suspension via cannulation. The amino acid dissolved over the course of approx. 1 h 30, forming a pale yellow solution. The solution was first filtered through a Buchner fitted with a Whatman paper #1 to remove any particles still in suspension. Then, the solution was concentrated by rotary evaporation, transferred to a beaker, and hexane was added to precipitate the product. The off-white solid was isolated by filtration and dissolved in anhydrous THF (˜600 mL). The solution was filtered over a bed of Celite to remove any insoluble material. An excess of hexane (˜6 L) was added to the filtrate to precipitate the product. The NCA was isolated by filtration and dried in vacuo. 114.05 g, 74.3% of Tyr(OBzl) NCA was isolated as a off-white powder. ¹H NMR (d₆-DMSO) δ 9.07 (1H), 7.49-7.29 (5H), 7.12-7.07 (2H), 6.98-6.94 (2H), 5.06 (2H), 4.74 (1H), 3.05-2.88 (2H) ppm.

### Example 10

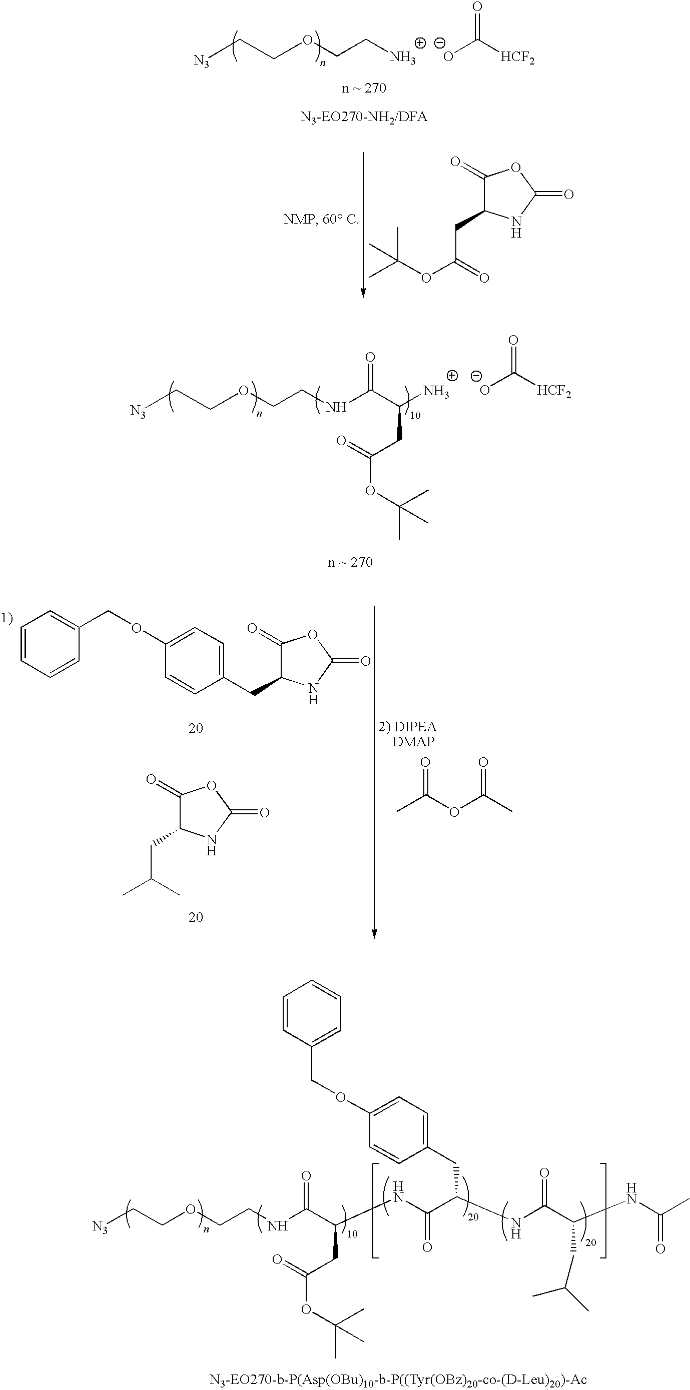

### N₃-Poly(ethylene oxide)₂₇₀-b-Poly(Asp(OBu)₁₀)-b-Poly(dLeu₂₀-co-Tyr(OBzl)₂₀)-Ac

Step A: DFA^{- +}NH₃-Poly(ethylene oxide)₂₇₀-N₃ (294 g, 25.6 mmol) from Example 6 was weighed into an oven-dried, 6 L jacketed round-bottom flask, dissolved in toluene (2 L), and dried by azeotropic distillation. After distillation, the polymer was left under vacuum overnight before adding the NCA. Asp(OBu) NCA (55 g, 256 mmol) from Example 8 was added to the flask, the flask was evacuated under reduced pressure, and subsequently backfilled with nitrogen gas. Dry N-methylpyrrolidone (NMP) (1.8 L) was introduced by cannula and the solution was heated to 60° C. The reaction mixture was allowed to stir for 48 hours at 60° C. under nitrogen gas.

Step B: D-Leu NCA (82 g, 0.522 mol) (Example 7) and Tyr (OBzl) NCA (155 g, 0.522 mol) (Example 9) were dissolved under nitrogen gas into 360 ml of NMP into an oven-dried, round bottom flask and the mixture was subsequently cannulated to the polymerization reaction via a syringe. The solution was allowed to stir at 60° C. for another three days and 12 hrs at which point the reaction was complete (by HPLC). The solution was cooled to room temperature and 25 mL were precipitated into 1 L of ether.

Step C: Diisopropylethylamine (DIPEA) (50 mL), dimethylaminopyridine (DMAP) (5 g), and acetic anhydride (50 mL) were added to the rest of the solution. Stirring was continued overnight at room temperature. The polymer was precipitated into diethyl ether (50 L) and isolated by filtration. The title product was isolated by filtration and dried in vacuo to give the block copolymer as an off-white powder (426 g, Yield=73%). ¹H NMR (d₆-DMSO) δ 8.43-7.62 (50H), 7.35 (100H), 7.1 (40H), 6.82 (40H), 4.96 (40H), 4.63-3.99 (50H), 3.74-3.2 (1500H), 3.06-2.6 (60H), 1.36 (90H), 1.27-0.47 (180).

### Example 11

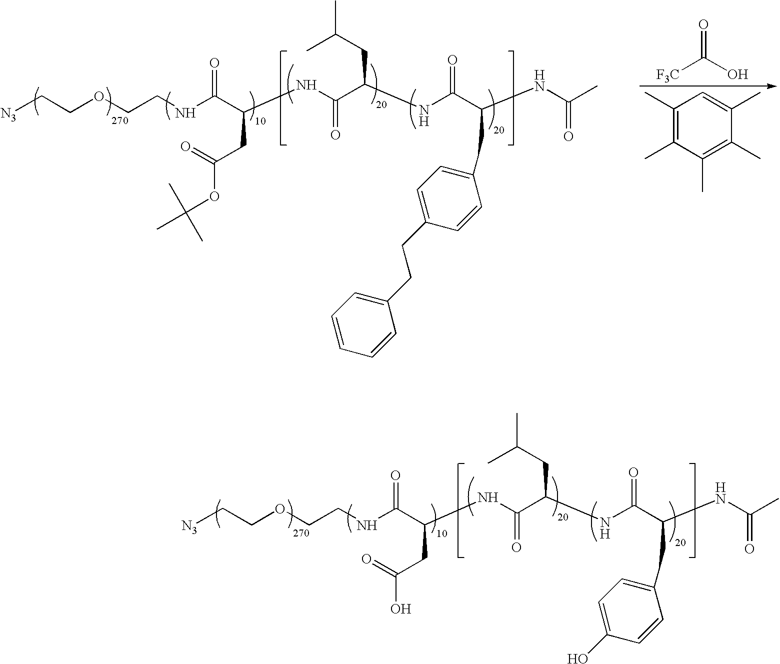

N₃-Poly(ethylene oxide)₂₇₀-b-Poly(Asp₁₀)-b-Poly(dLeu₂₀-co-Tyr₂₀)-Ac N₃-Poly(ethylene oxide)₂₇₀-b-Poly(Asp(OBu)₁₀)-b-Poly(dLeu₂₀-co-Tyr(OBzl)₂₀)-Ac (420 g, 20.5 mmol) from Example 10 was dissolved into 3 L of a solution of pentamethyl benzene (PMB, 0.5M) in trifluoroacetic acid (TFA). The reaction was allowed to stir for five hours at room temperature. The solution was precipitated into diethyl ether (50 L) and the solid was recovered by filtration through a 2 L medium frit. The polymer was redissolved into 4 L of dichloromethane and precipitated into diethyl ether (˜50 L). The polymer was redissolved one more time into a 50:50 dichloromethane:isopropanol mixture and diethyl ether was poured on the top of the solution (˜50 L). The title compound was obtained as an off-white polymer after drying the product overnight in vacuo (309.3 g, 83% yield). ¹H NMR (d₆-DMSO) δ 12.2 (2H), 9.1 (13H), 8.51-7.71 (49H), 6.96 (29H), 6.59 (26H), 4.69-3.96 (59H), 3.81-3.25 (1040H), 3.06-2.65 (45H), 1.0-0.43 (139). ¹³C NMR (d₆-DMSO) δ 171.9, 171, 170.5, 170.3, 155.9, 130.6, 129.6, 127.9 115.3, 114.3, 70.7, 69.8, 54.5, 51.5, 50, 49.8, 49.4, 36.9, 36, 24.3, 23.3, 22.3, 21.2. IR (ATR) 3290, 2882, 1733, 1658, 1342, 1102, 962 cm⁻¹. Mₙ (MALDI-TOF)=17,300 g/mol. PDI (DMF GPC)=1.1

### Example 12

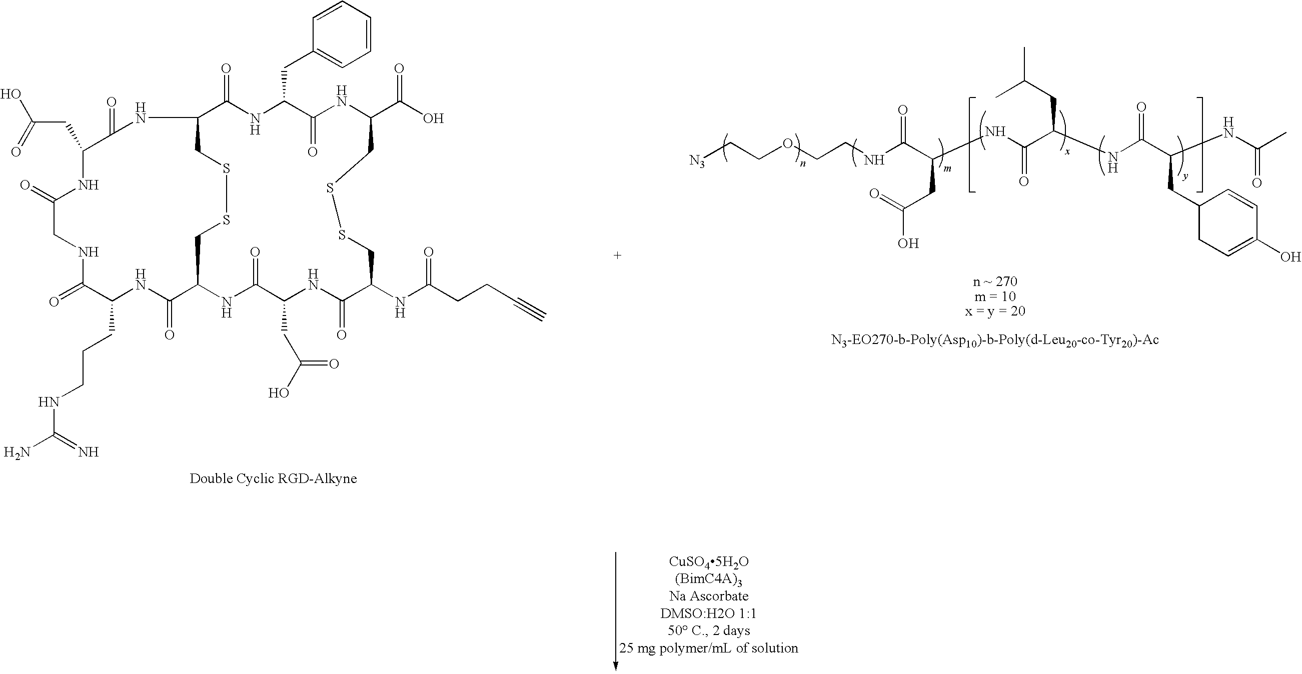

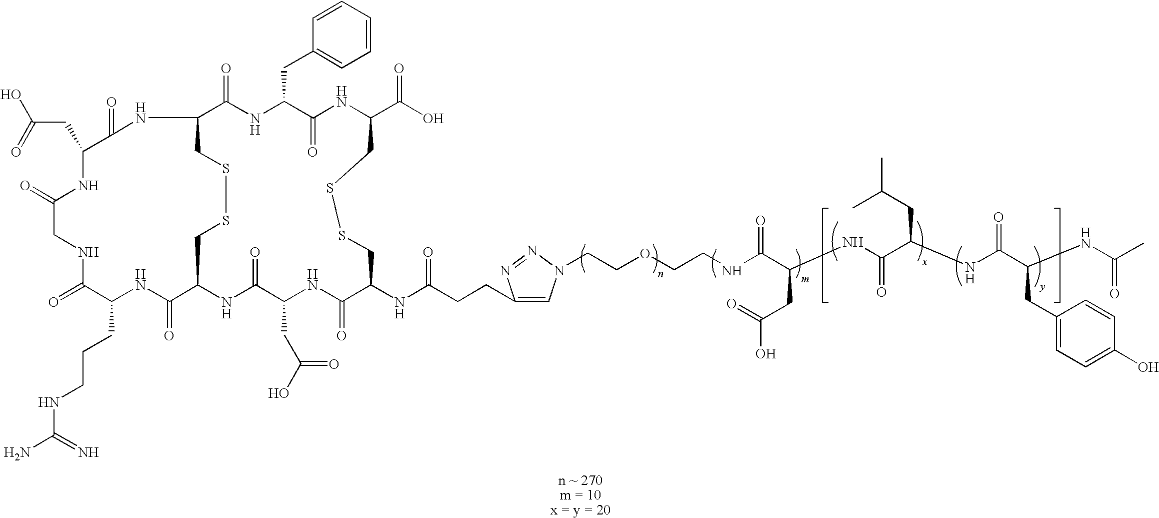

Double Cyclic RGD-Poly(ethylene oxide)₂₇₀-b-Poly(Asp₁₀)-b-Poly(dLeu₂₀-co-Tyr₂₀)-Ac N₃-Poly(ethylene oxide)₂₇₀-b-Poly(Asp₁₀)-b-Poly(dLeu₂₀-co-Tyr₂₀)-Ac from Example 11 (512.1 mg, 27.6 μmol), double cyclic RGD-alkyne (37.1 mg, 33.8 μmol), sodium ascorbate (145.7 mg, 0.73 mmol), (BimC4A)3 (Finn, M. G. et. al. J. Am. Chem. Soc. 2007, 129, 12696-12704) (43.1 mg, 60.8 μmol), Cu50₄. 5H₂O (6.77 mg, 27.1 μmol), DMSO (10 mL) and water (10 mL) were added into a 20 mL vial, capped and stirred for 48 hr at 50° C. The light brown solution was dialyzed (3500 MWCO bag) 3 times against deionized water with EDTA (15 g/L) and 2 times against deionized water. The solution was freeze-dried and the title compound was obtained as an off-white powder. (448.1 mg, 82% yield). ¹H NMR (D₂O) δ 8.16 (1H), 7.87 (1H), 7.44-6.72 (10H), 4.35 (4H), 4.06-3.41 (1040H), 3.27-2.73 (14H).

### Example 13

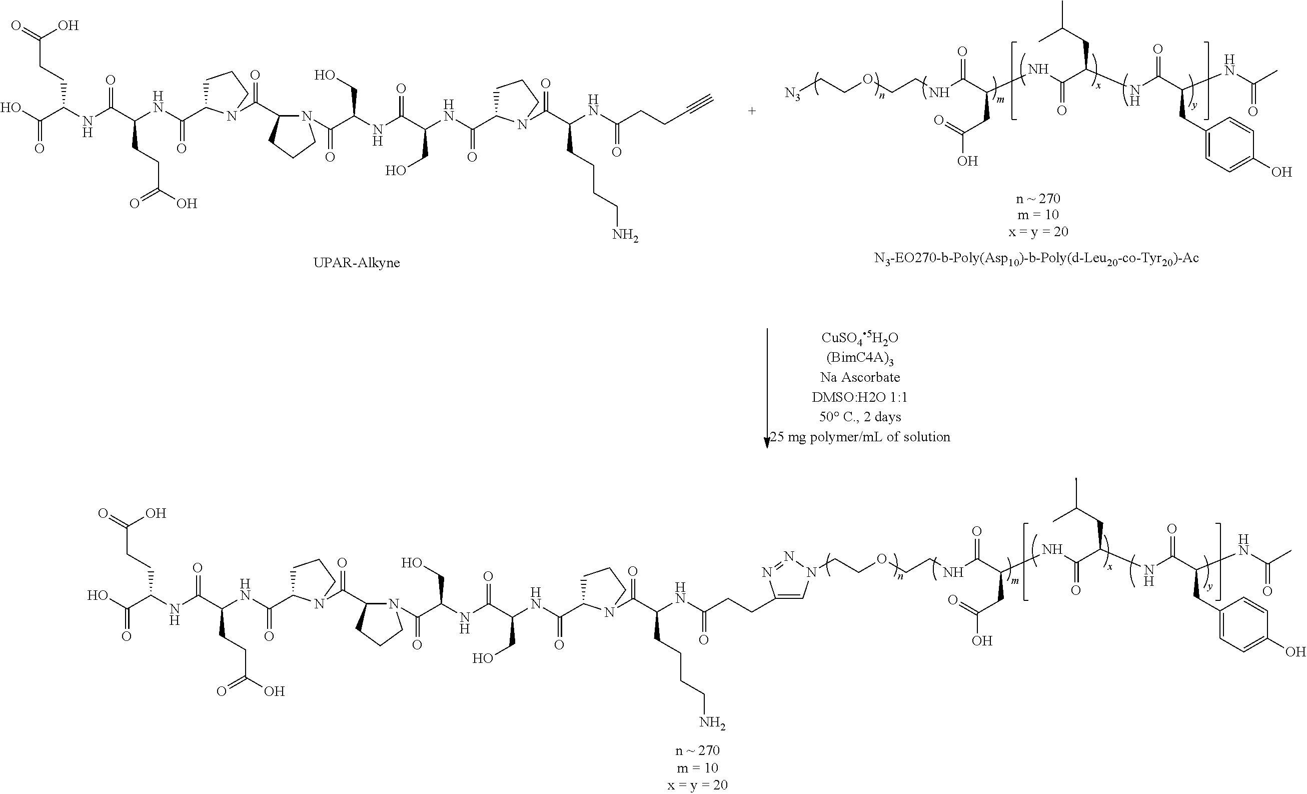

UPAR-Poly(ethylene oxide)₂₇₀-b-Poly(Asp₁₀)-b-Poly(dLeu₂₀-co-Tyr₂₀)-Ac N₃-Poly(ethylene oxide)₂₇₀-b-Poly(Asp₁₀)-b-Poly(dLeu₂₀-co-Tyr₂₀)-Ac (306.2 mg, 16.4 μmol) from Example 11, alkynyl-UPAR (25.0 mg, 21.1 μmol), sodium ascorbate (86.9 mg, 0.44 mmol), (BimC4A)3 (23.4 mg, 33.1 μmol), CuSO₄. 5H₂O (5.44 mg, 21.8 μmol), DMSO (6 mL) and water (6 mL) were added into a 20 mL vial, capped and stirred for 48 hr at 50° C. The light brown solution was dialyzed (3500 MWCO bag) 3 times against deionized water with EDTA (15 g/L) and 2 times against deionized water. The solution was freeze-dried and the title compound was obtained as an off-white powder. (272.2 mg, 85% yield). ¹H NMR (D₂O) δ 8.16 (1H), 7.84 (1H), 7.44-6.72 (4H), 4.60-4.26 (18H), 4.06-3.41 (1040H), 2.99 (6H), 2.73 (1H), 2.58-1.69 (34H).

### Example 14

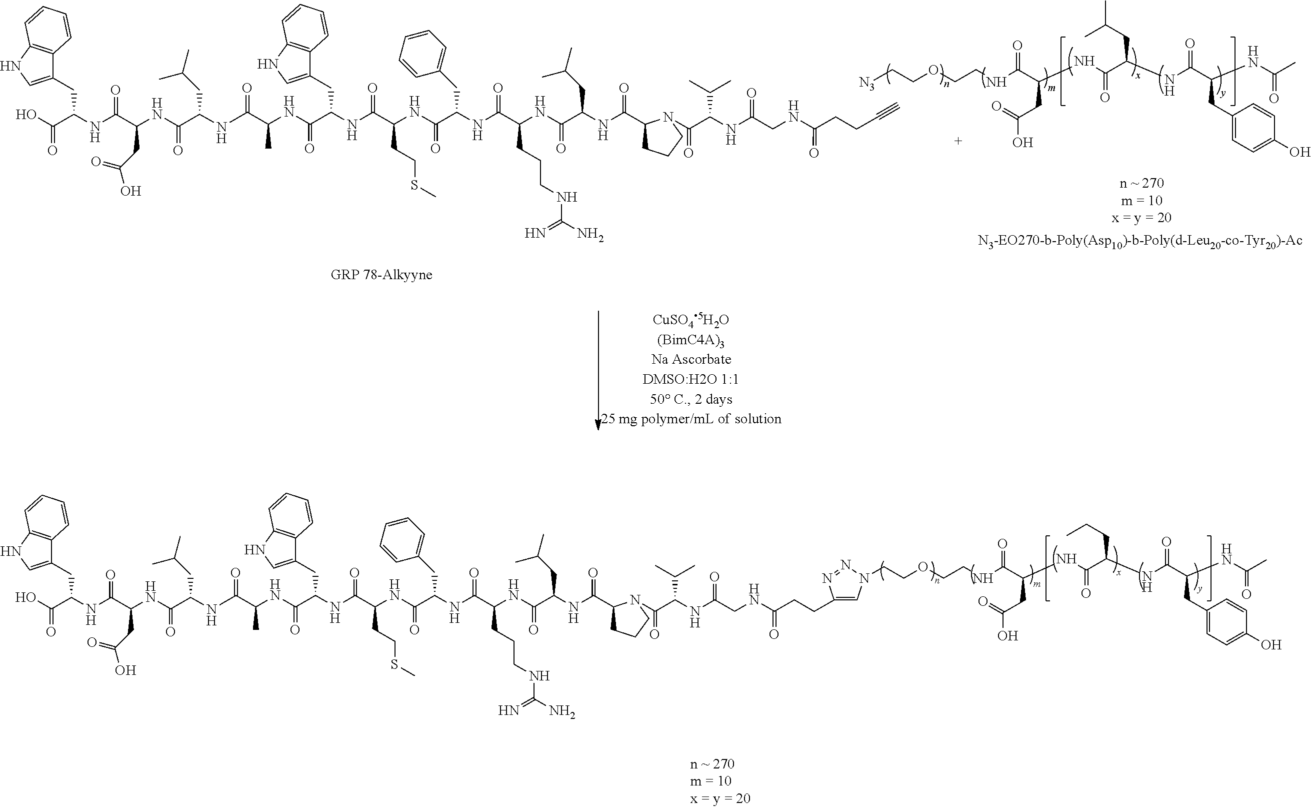

GRP78-Poly(ethylene oxide)₂₇₀-b-Poly(Asp₁₀)-b-Poly(dLeu₂₀-co-Tyr₂₀)-Ac N₃-Poly(ethylene oxide)₂₇₀-b-Poly(Asp₁₀)-b-Poly(dLeu₂₀-co-Tyr₂₀)-Ac (296.6 mg, 15.9 μmol) from Example 11, alkynyl-GRP 78 (32.5 mg, 20.7 μmol), sodium ascorbate (80.55 mg, 0.41 mmol), (BimC4A)3 (24.8 mg, 35 μmol), CuSO₄. 5H₂O (5.30 mg, 21.2 μmol), DMSO (6 mL) and water (6 mL) were added into a 20 mL vial, capped and stirred for 48 hr at 50° C. The light brown solution was dialyzed (3500 MWCO bag) 3 times against DI water with EDTA (15 g/L) and 2 times against DI water. The solution was freeze-dried and an off-white powder was obtained. (244.3 mg, 92% yield). ¹H NMR (D₂O) δ 8.16 (1H), 7.44-6.72 (8H), 4.35 (3H), 4.06-3.41 (1040H), 2.97-2.62 (12H).

### Example 15

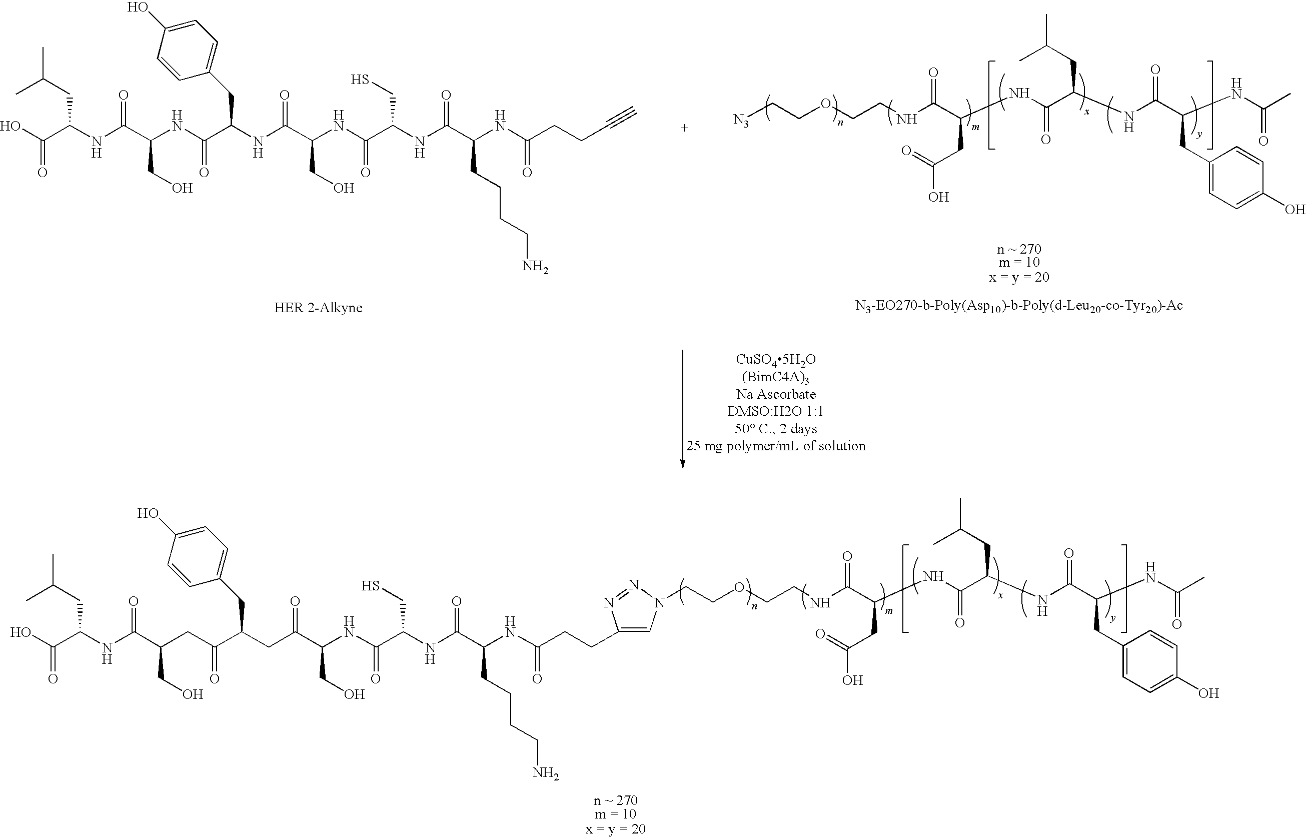

HER2-Poly(ethylene oxide)₂₇₀-b-Poly(Asp₁₀)-b-Poly(dLeu₂₀-co-Tyr₂₀)-Ac N₃-Poly(ethylene oxide)₂₇₀-b-Poly(Asp₁₀)-b-Poly(dLeu₂₀-co-Tyr₂₀)-Ac (299.3 mg, 16 μmol) from Example 11, alkynyl-HER 2 (26.2 mg, 32.9 μmol), sodium ascorbate (79.8 mg, 0.402 mmol), (BimC4A)3 (23.13 mg, 32.6 μmol), CuSO₄. 5H₂O (3.94 mg, 15.8 μmol), DMSO (6 mL) and water (6 mL) were added into a 20 mL vial, capped and stirred for 48 hr at 50° C. The light brown solution was dialyzed (3500 MWCO bag) 3 times against DI water with EDTA (15 g/L) and 2 times against DI water. The solution was freeze-dried and an off-white powder was obtained. (300 mg, 96% yield). ¹H NMR (D₂O) δ 8.16, 7.09, 6.82, 4.35, 4.06-3.41 (1040H), 3.27-2.73 (14H).

### Example 16

### CMC Determination

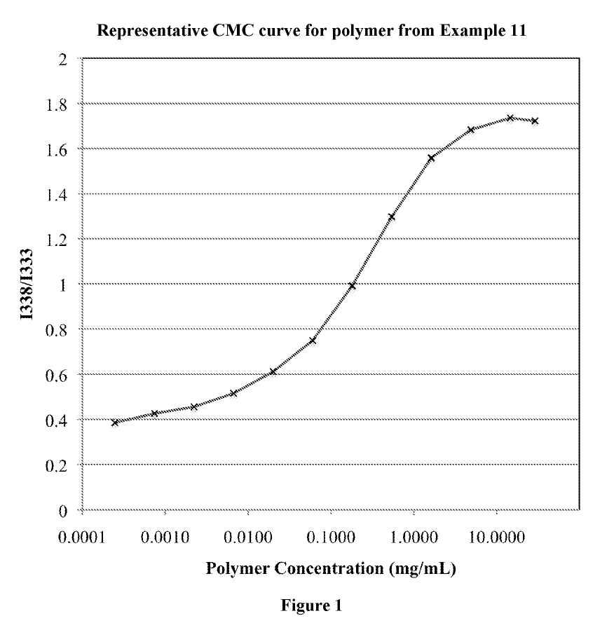

The CMC of micelles prepared from block copolymers, as described above, were determined using the method described by Eisnberg. (Astafieva, I.; Zhong, X. F.; Eisenberg, A. “Critical Micellization Phenomena in Block Copolymer Polyelectrolyte Solutions” Macromolecules 1993, 26, 7339-7352.) To perform these experiments, a constant concentration of pyrene (5×10⁻⁷ M) was equilibrated with varying concentrations of block copolymer (ca. 2×10²-1×10⁻⁴ mg/mL) in phosphate buffered saline at room temperature for 16 hours. Excitation spectra (recorded on a Perkin Elmer LS-55 spectrophotometer with excitation between 328 and 342 nm, emission at 390 nm, 2.5 nm slit width, 15 nm/min scan speed) were recorded for each polymer concentration and the fluorescence intensities recorded at 333 and 338 nm. Eisenberg has shown that the vibrational fine structure of pyrene is highly sensitive to the polarity of its environment. Specifically, the (0,0) excitation band of pyrene will shift from 333 nm in an aqueous environment to 338.5 nm in a hydrophobic environment. The ratio of peak intensities (I₃₃₈/I₃₃₃) reveals the hydrophobicity of the environment surrounding the pyrene. Values of ˜2.0 correspond to a hydrophobic environment such as polystyrene or poly(benzyl glutamate), whereas values of ˜0.35 correspond to an aqueous environment. Plotting this ratio vs. log of the block copolymer concentration allows for the graphical interpretation of the CMC value. A more quantitative number can be obtained by fitting a logarithmic (y=a ln(x)+b) regression to the data points between the two plateaus (at ˜2 and ˜0.35). The CMC can be found by setting y=0.35 and solving for x (concentration in mg/mL). FIG. 1 shows the resulting CMC curve for the copolymer prepared in Example 11 with the CMC found to be 0.2 mg/mL.

### Example 17

### Preparation of IT-141: SN-38 Encapsulation with Bath Sonication

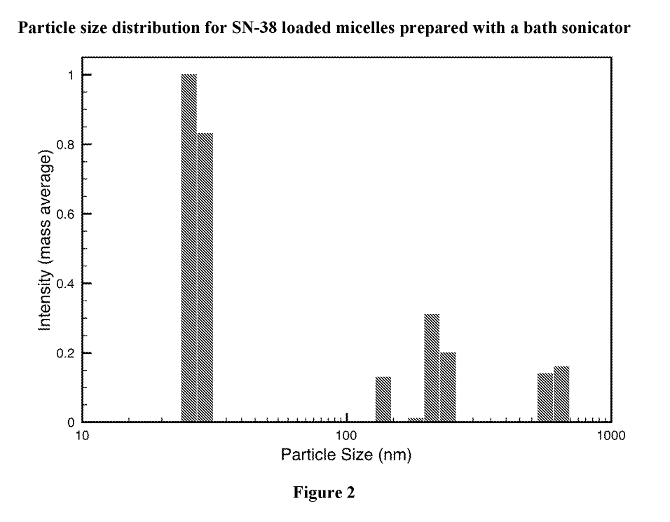

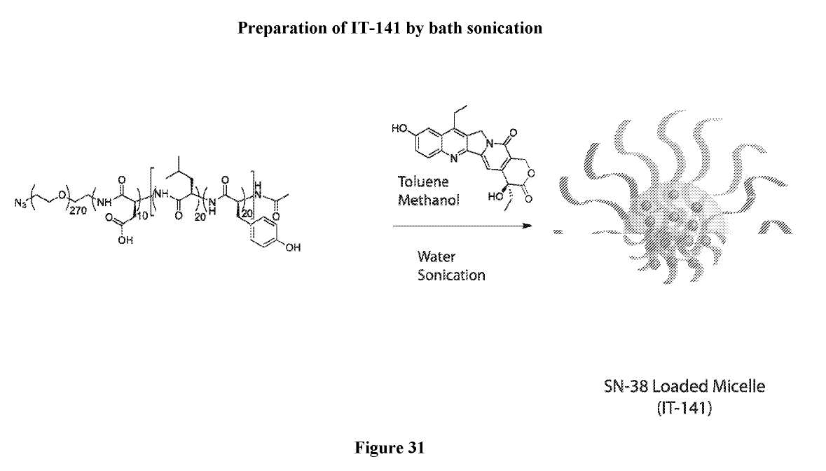

IT-141 N₃-Poly(ethylene oxide)₂₇₀-b-Poly(Asp₁₀)-b-Poly(dLeu₂₀-co-Tyr₂₀)-Ac (500 mg) from Example 11 and SN-38 (25 mg) were weighed into a 125 ml Erlenmeyer flask, dissolved in toluene (35 mL) and methanol (15 mL), then stirred, sonicated and heated until homogeneous. Separately, water (100 mL) was added to a 500 mL beaker, then the beaker submerged in the sonicating water bath (Fisher Scientific). An overhead stirrer was submerged in the beaker, set to stir at 900 RPM, then the sonicator turned on. The organic solution was then added drop-wise to the beaker resulting in a milk-like emulsion. The solution was stirred and sonicated for 4 hours until clear, centrifuged at 2400 rpm for 10 minutes, filtered through a 0.45 mm filter then lyophilized. A yellow powder (400 mg, 72% yield) was recovered after lyophilization. HPLC showed that the yellow powder contained 2% SN-38 by weight for a loading efficiency of 42%. See FIG. 31. Particle size distribution of IT-141 prepared by bath sonication is shown in FIG. 2.

### Example 18

### Preparation of IT-141: SN-38 Encapsulation with Probe Sonication

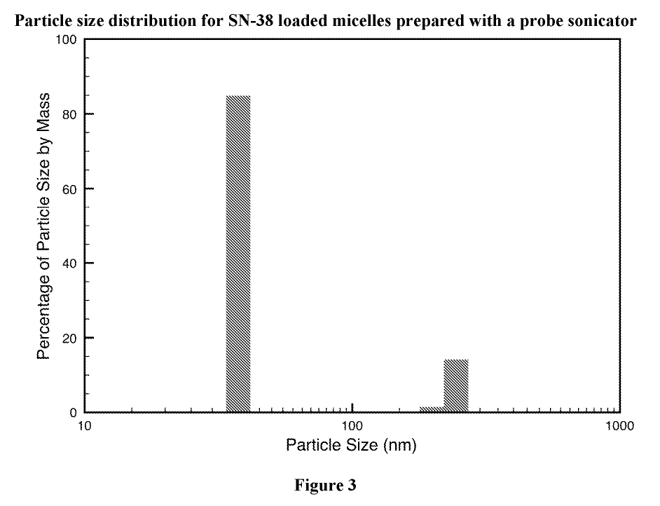

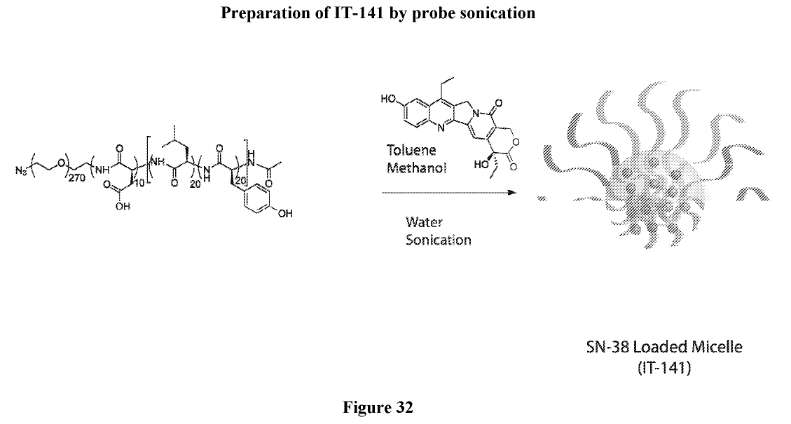

IT-141 N₃-Poly(ethylene oxide)₂₇₀-b-Poly(Asp₁₀)-b-Poly(dLeu₂₀-co-Tyr₂₀)-Ac (250 mg) from Example 11 and SN-38 (37.5 mg) were weighed into a 100 ml Erlenmeyer flask, dissolved in toluene (12 mL) and methanol (6 mL), then stirred, sonicated, and heated until homogeneous. Separately, water (200 mL) was added to a jacketed reaction flask containing a stir bar, with cooling fluid circulating at 16° C. A 1″ titanium sonication horn connected to a Misonix 4000 generator was submerged to a depth of 1.5″ in the water. The sonicator was turned on with 100% amplitude and the solution stirred. The organic solution was then added drop-wise to the reaction flask, resulting in a milk-like emulsion. The solution was stirred and sonicated for 1 hour then poured into a separate beaker. The resulting solution was allowed to stir at room temperature for 36 hours in a fume hood until the solution is opalescent. This solution was centrifuged at 2400 rpm for 10 minutes, filtered through a 0.45 mm filter then lyophilized. A yellow powder (220 mg, 77% yield) was recovered after lypholyzation. HPLC showed that the yellow powder contained 13% SN-38 by weight for a loading efficiency of 93%. See FIG. 32. Particle size distribution of IT-141 prepared by probe sonication is shown in FIG. 3.

### Example 19

### Preparation of IT-141: SN-38 Encapsulation with Silverson Shear Mixer

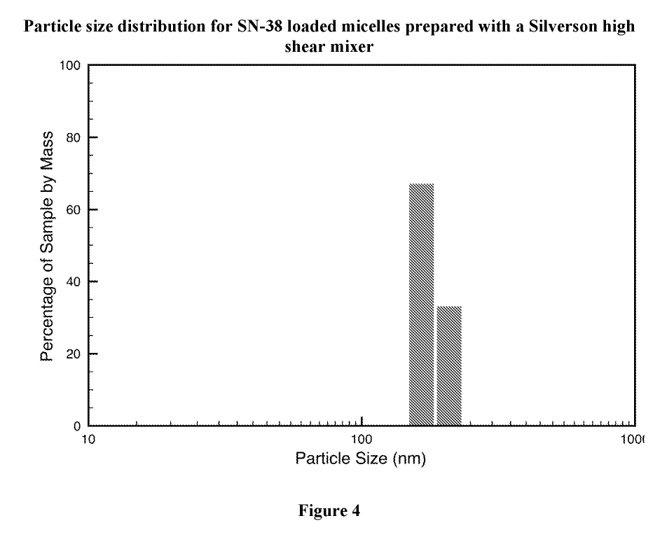

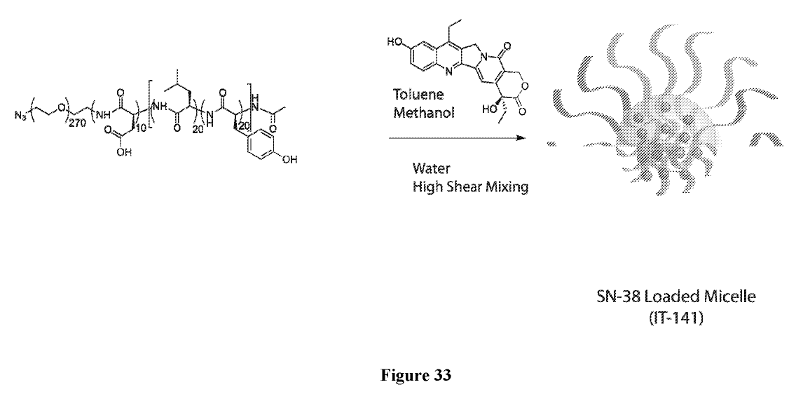

IT-141 N₃-Poly(ethylene oxide)₂₇₀-b-Poly(Asp₁₀)-b-Poly(dLeu₂₀-co-Tyr₂₀)-Ac (500 mg) from Example 11 and SN-38 (70 mg) were weighed into a 100 ml Erlenmeyer flask, dissolved in toluene (16 mL) and methanol (8 mL), then stirred, sonicated and heated until homogeneous. Separately, water (400 mL) was added to a jacketed reaction flask with cooling fluid circulating at 5° C. A Silverson high shear mixer equipped with a fine emulsion screen was submerged 1 inch from the bottom of the reaction beaker in the water. The mixer was turned to 10,000 rpm and the solution stirred. The organic solution was then added drop-wise to the reaction flask, resulting in a milk-like emulsion. The solution was mixed for 1 hour then poured into a separate beaker. The resulting solution was allowed to stir at room temperature for 36 hours in a fume hood until the solution was opalescent. This solution was centrifuged at 2400 rpm for 10 minutes, filtered through a 0.45 mm filter then lyophilized. A yellow powder (400 mg, 72% yield) was recovered after lypholyzation. HPLC showed that the yellow powder contained 11.3% SN-38 by weight for a loading efficiency of 93% efficient. See FIG. 33. Particle size distribution of IT-141 prepared by Silverson shear mixing is shown in FIG. 4. The resulting micelle diameter was 138 nm with a standard deviation of 14 nm.

### Example 20

### SN-38 Loaded, RGD Targeted Micelles

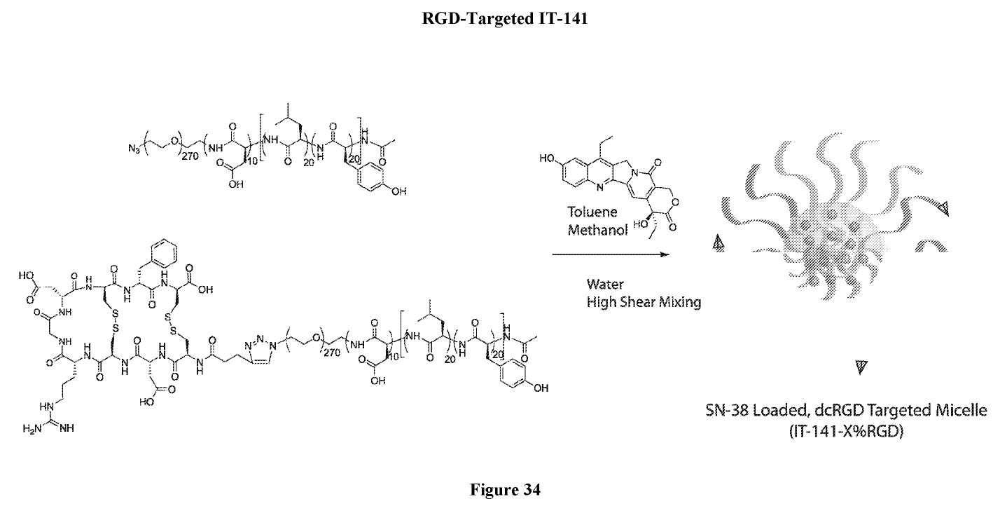

RGD-targeted IT-141 is prepared from double cyclic RGD-poly(ethylene oxide)₂₇₀-b-poly(Asp₁₀)-b-poly(dLeu₂₀-co-Tyr₂₀)-Ac, prepared according to Example 12, and SN-38 according to the method of Example 19 to form the micelles depicted in FIG. 34.

### Example 21

### SN-38 Loaded, HER2 Targeted Micelles

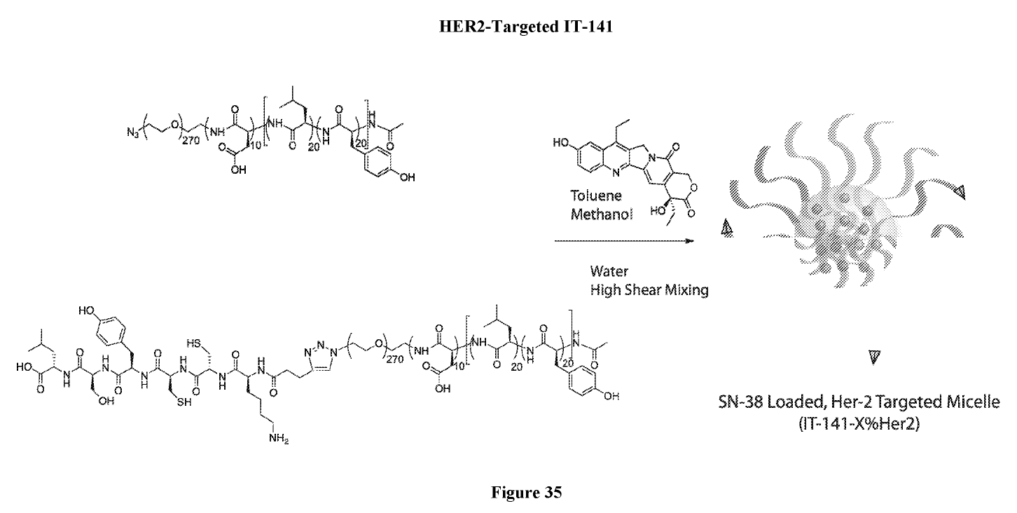

HER2-targeted IT-141 is prepared from HER2-poly(ethylene oxide)₂₇₀-b-poly(Asp₁₀)-b-poly(dLeu₂₀-co-Tyr₂₀)-Ac, prepared according to Example 15, and SN-38 according to the method of Example 19 to form the micelles depicted in FIG. 35.

### Example 22

### SN-38 Loaded, uPAR Targeted Micelles

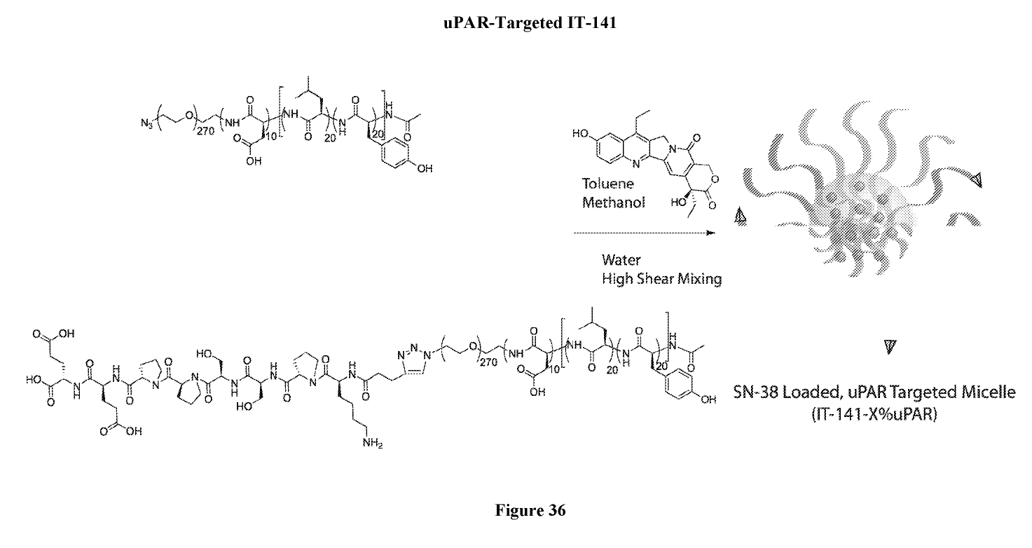

uPAR-targeted IT-141 is prepared from UPAR-poly(ethylene oxide)₂₇₀-b-poly(Asp₁₀)-b-poly(dLeu₂₀-co-Tyr₂₀)-Ac, prepared according to Example 13, and SN-38 according to the method of Example 19 to form the micelles depicted in FIG. 36.

### Example 23

### SN-38 Loaded, GRP78 Targeted Micelles

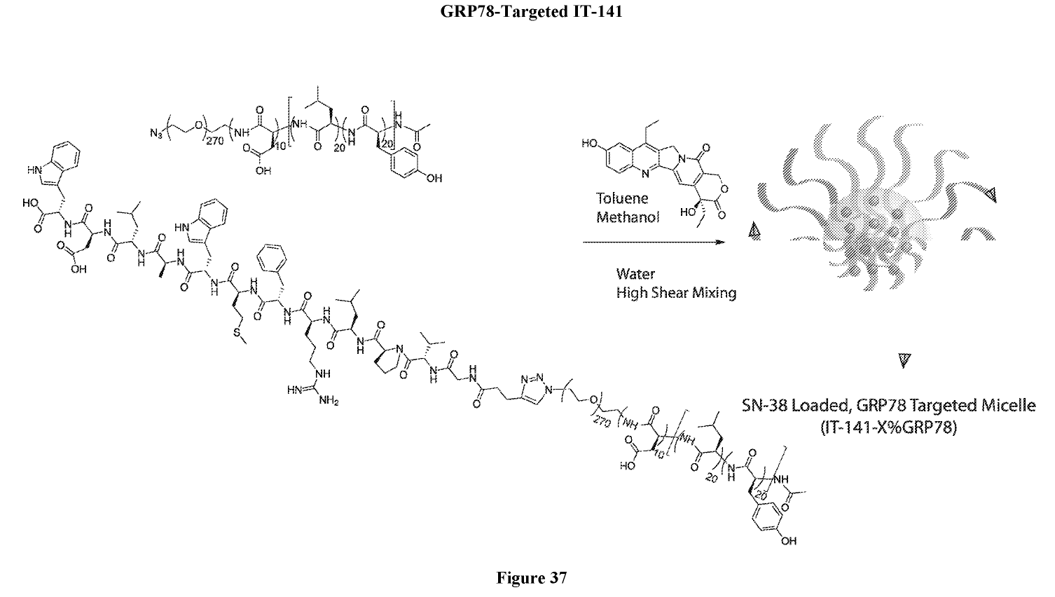

GRP78-targeted IT-141 is prepared from GRP78-poly(ethylene oxide)₂₇₀-b-poly(Asp₁₀)-b-poly(dLeu₂₀-co-Tyr₂₀)-Ac, prepared according to Example 13, and SN-38 according to the method of Example 19 to form the micelles depicted in FIG. 37.

### Example 24

### Cytotoxicity of Polymer Micelles

MCF-7, BT474, LNCaP, and MG-63 cells were maintained in RPMI 1640 supplemented with 10% FBS, 2 mM L-glutamine, 100 IU penilcillin/mL and 100 μg/mL streptomycin/mL. MDA-MB-231 and Saos2 cells were maintained in DMEM with 10% FBS, 2 mM L-glutamine 100 IU penicillin/mL and 100 μg/mL streptomycin/mL. MCF10A cells were maintained in a 50:50 mix of DMEM and Ham's F12 supplemented with 5% FBS, 2 mM L-glutamine, 10 ng/mL EGF, 500 ng/mL hydrocortisone, 0.01 mg/mL insulin, 100 IU penicillin/mL and 100 μg/mL streptomycin/mL. Cells were maintained at 37 degrees Celsius with 5% CO2 and were subcultured weekly. All other cell lines were cultured according to ATCC guidelines.

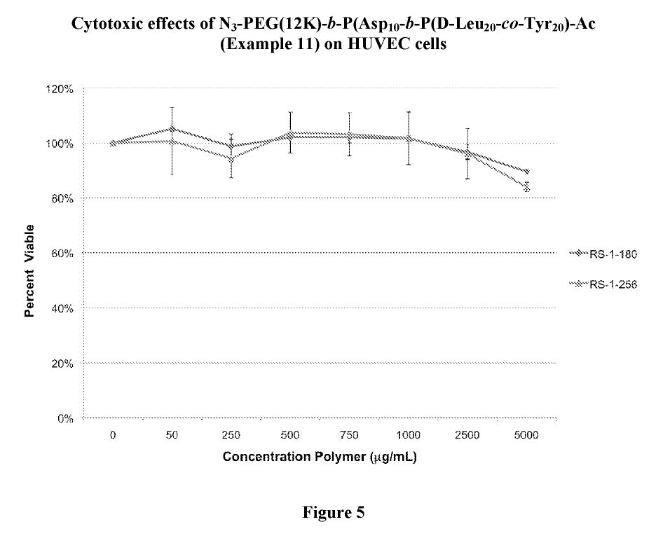

1.2×10⁴ HUVEC cells were plated in 96-well plates. Twenty-four hours later, media was replaced with test micelle diluted in growth media at a final concentration of 0, 100, 250, 500, 750, 1000, 2500 or 5000 μg/mL poly(ethylene oxide)₂₇₀-b-poly(Asp₁₀)-b-poly(dLeu₂₀-co-Tyr₂₀)-Ac from Example 11 (two separated batches of identical polymer were used). After 72 hours, cell viability was determined using the Cell-Titer Glo reagent according to the manufacturer's protocol (Promega, Madison, Wisc.). Data were collected using a plate reader with luminescence detection (BMG Labtech, Durham, N.C.). Experiments were performed in triplicate. As depicted in FIG. 5, cell viability was greater than 85% even for the highest concentration of polymer administered.

### Example 25

### Cytotoxicity of SN-38 Loaded Micelles

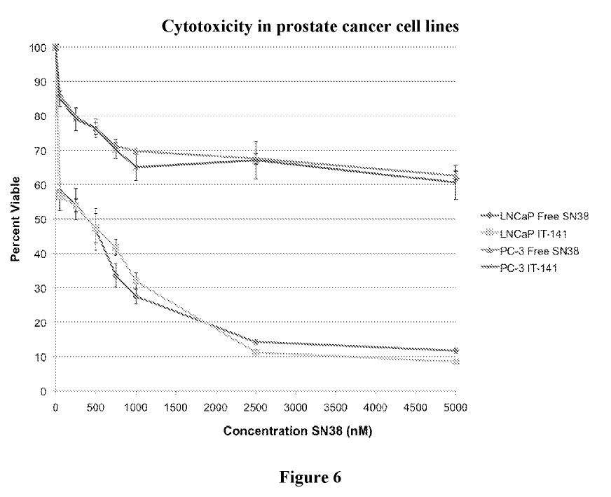

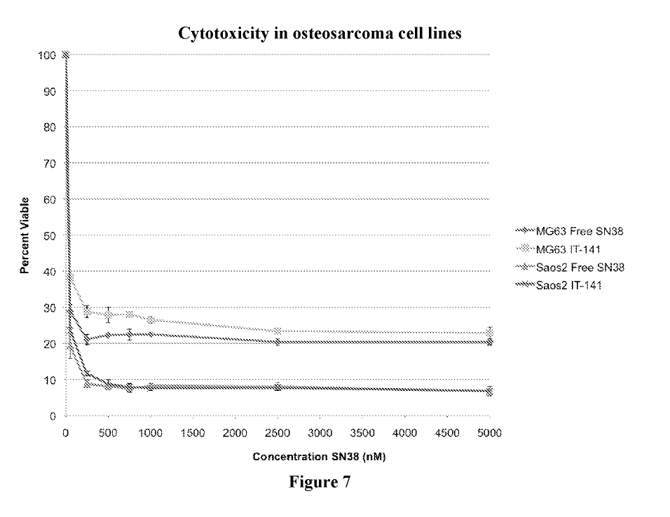

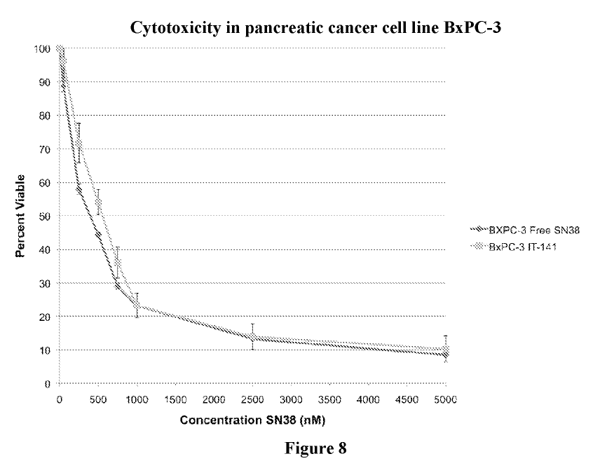

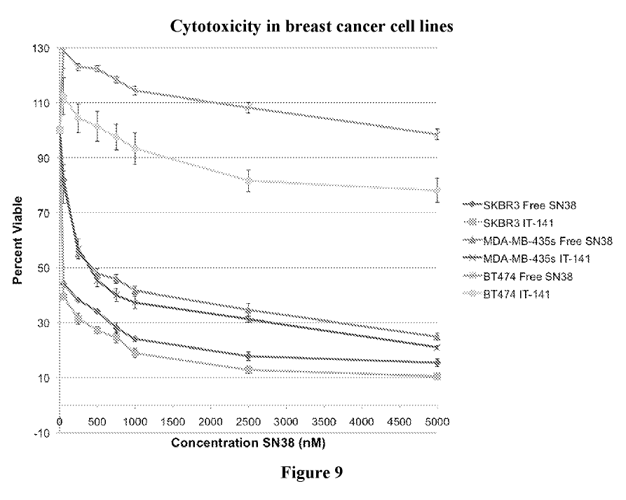

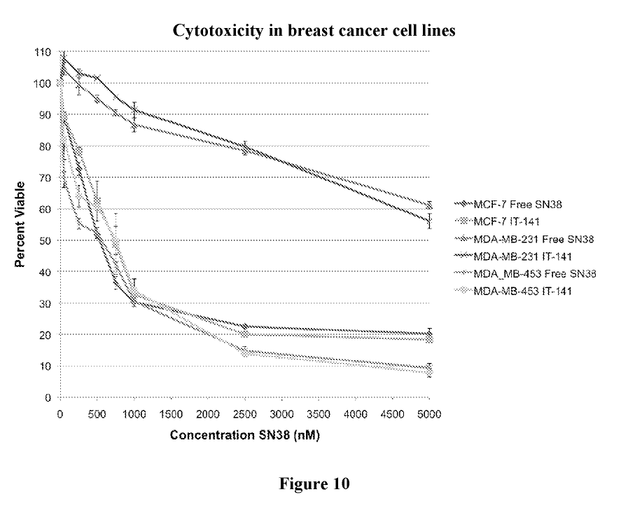

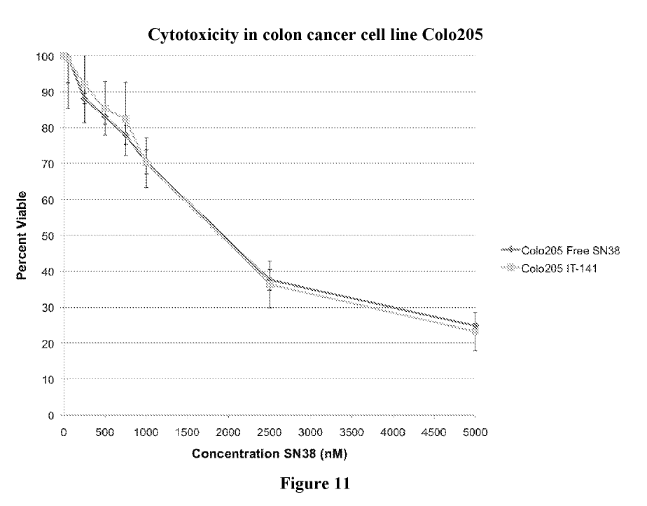

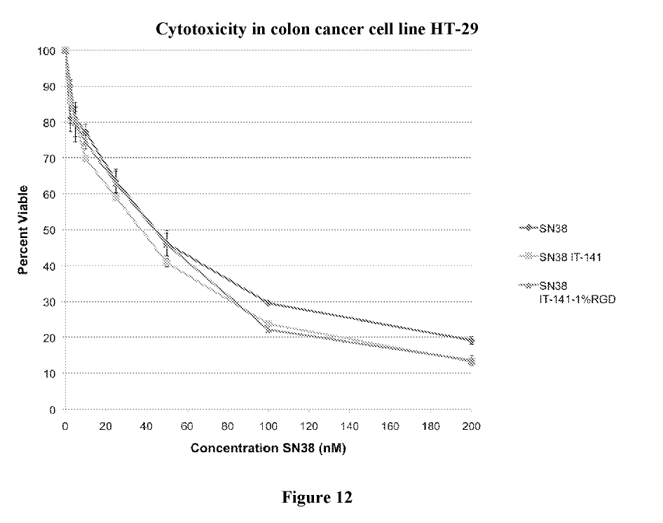

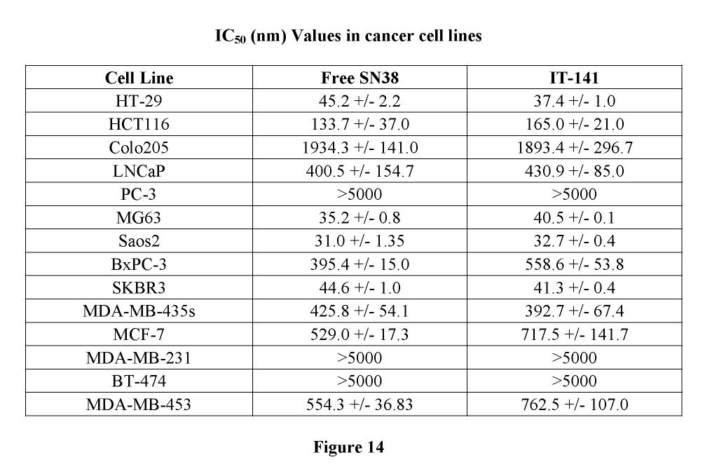

Using the method described in Example 24, approximately 1.2×10⁴ cells of the desired cell line were plated in 96-well plates. Twenty-four hours later, media was replaced with micelle diluted in growth media at a final concentration of 0, 100, 250, 500, 750, 1000, 2500 or 5000 nM SN-38 administered as free SN-38 in DMSO or encapsulated in a polymer micelle comprising poly(ethylene oxide)₂₇₀-b-poly(Asp₁₀)-b-poly(dLeu₂₀-co-Tyr₂₀)-Ac and SN-38 (also referred to as IT-141, from Example 19). After 72 hours, cell viability was determined using the Cell-Titer Glo reagent according to the manufacturer's protocol (Promega, Madison, Wisc.). Data were collected using a plate reader with luminescence detection (BMG Labtech, Durham, N.C.). Experiments were performed in triplicate. The results are depicted in FIG. 6 for prostate cancer cell lines, FIG. 7 for osteosarcoma cell liens, FIG. 8 for pancreatic cancer cell lines, FIGS. 9 and 10 for breast cancer cell lines, and FIGS. 11, 12, and 13 for colon cancer cell lines. IC₅₀ values for these results treatments are summarized in table format in FIG. 14.

### Example 26

### S-Phase Arrest Experiments

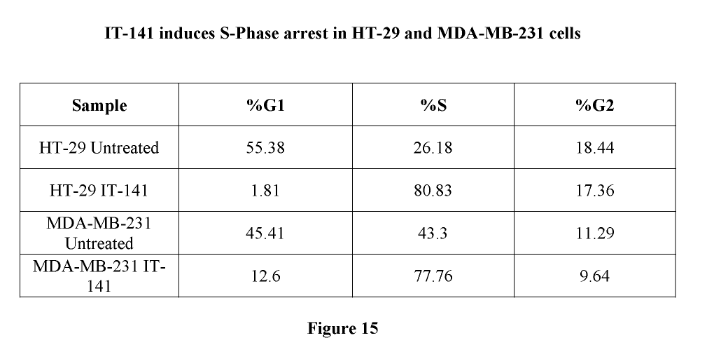

Cells were plated in 60 mm tissue culture dishes and treated with or without 10 μM IT-141, prepared according to Example 19, for 24 hours. Cells were harvested and fixed cold 70% ethanol with vortexing. Cells were washed with PBS and cells were suspended in 40 μg/mL propidium iodide with 100 μg/mL RNAse A for twenty minutes. Single-cells were analyzed for DNA content by flow cytometry. The data are depicted in FIG. 15 and demonstrate an increase in the percentage of cells in S-phase following treatment with IT-141 (Example 19).

### Example 27

### Blocking Experiments

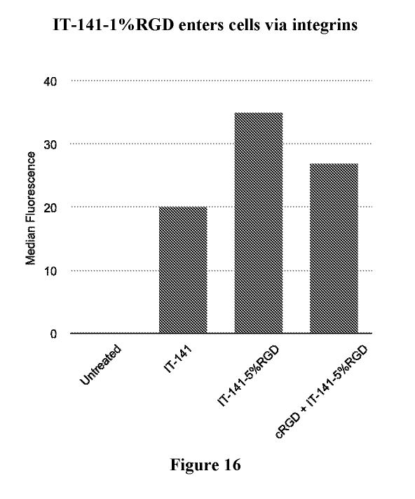

MDA-MB-435s cells were analyzed by flow cytometry for intracellular fluorescence of SN38 following treatment with IT-141-5% RGD. Cells were treated with either 60 μM IT-141 (Example 19), 60 μM IT-141-5% RGD (Example 20), or pre-treated with 750 μg/mL free cRGD peptide prior to treatment with 60 μM IT-141-5% RGD. Ninety minutes after treatment with micelles, cells were harvested and analyzed using an LSR II flow cytometer for SN38 fluorescence. SN38 was excited using a violet laser and emission was detected with a 575/26 bandpass filter. The data shown in FIG. 16 illustrates that cRGD peptide partially inhibits entry of IT-141-5% RGD into MDA-MB-435s cells.

### Example 28

### MTD Study Comparing IT-141 to IT-141-1% RGD in Tumor-Bearing Nude Mice

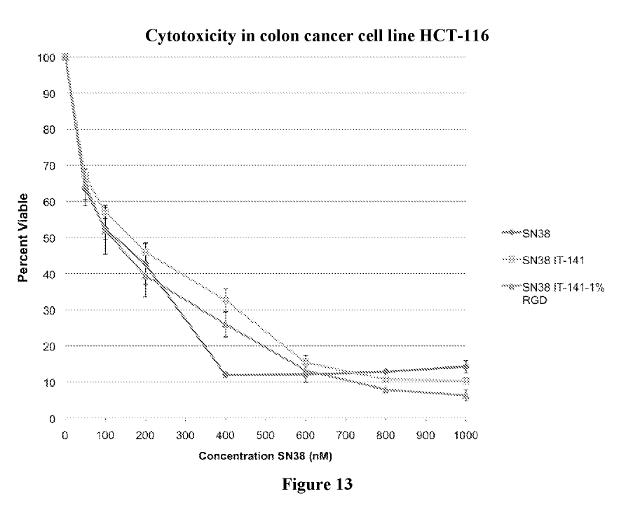

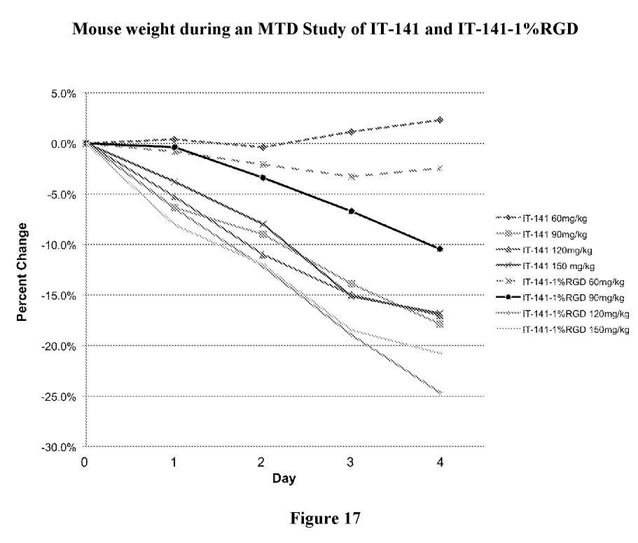

HT-29 cells were injected subcutaneously in nude mice and grown to 100 mm³. Mice were segregated into groups and injected intravenously with varying doses (60 to 150 mg/kg) of IT-141 (Example 19) or IT-141-1% RGD (Example 20). Mice were weighed for four days. Results in FIG. 17 are displayed as the percent change in average body weight from Day 0 in FIG. 13. Seven days after injection with IT-141, 2/4 mice at 150 mg/kg, 4/6 at 120 mg/kg, 5/6 at 90 mg/kg, and 0/6 at 60 mg/kg died. Seven days after injection of IT-141-1% RGD, 5/6 mice at 150 mg/kg, 5/6 at 120 mg/kg, 2/6 at 90 mg/kg, and 0/6 at 60 mg/kg died.

### Example 29

### Study Comparing the MTD of IT-141 in Tumor-Bearing Nude Mice and Healthy CD-1 Mice

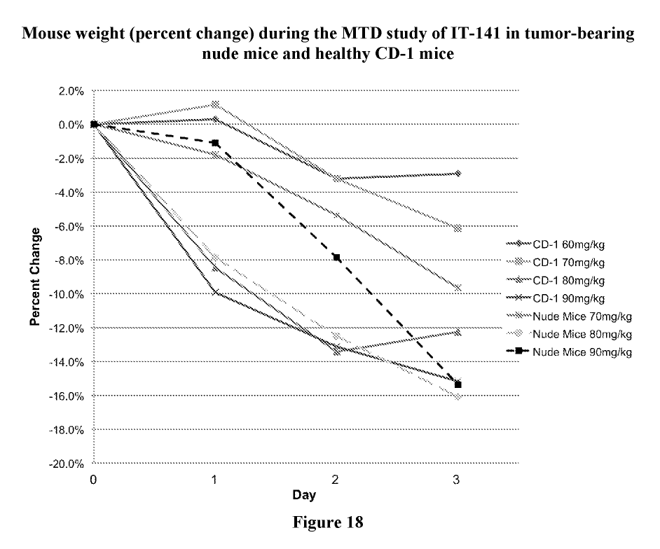

HT-29 cells were injected subcutaneously in nude mice and grown to 100 mm³. Both CD-1 mice and tumor bearing mice were segregated into groups and injected intravenously with varying doses (60 to 90 mg/kg) of IT-141. Mice were weighed for three days. Results displayed in FIG. 18 are displayed as the percent change in average body weight from Day 0 in FIG. 14. Seven days after injection with IT-141, 6/6 CD-1 mice at 90 mg/kg, 5/6 at 80 mg/kg, 4/6 at 70 mg/kg, and 1/3 at 60 mg/kg died. In comparison, 5/6 nude mice at 90 mg/kg, 3/6 at 80 mg/kg, and 3/6 at 70 mg/kg died.

### Example 30

### Antitumor Efficacy of IT-141

HT-29 colon cancer cells were cultured according to ATCC guidelines, harvested by trypsin incubation, and resuspended at a concentration of 2 million cells per 0.1 mL in saline for injection. Mice were inoculated by injecting 0.1 mL (i.e. 2 million cells) subcutaneously into the right flanks of the mice.

When tumors reached approximately 100 mm³ the mice were randomized into treatment groups. Mice were dosed by a fast IV bolus into the tail vein; the injection volume was 0.2 mL. Tumors were measured by digital caliper, and volume (mm³) was calculated using the formula V=(W²×L)/2, where width (W) is the largest diameter measurement and length (L) is the diameter measurement perpendicular to the width. The dosing schedule was once every four days for a total of 3 injections over 8 days. The vehicle for polymer delivery was isotonic saline.

Clinical observations during the study included changes in mouse body weight, morphological observations of sick mouse syndrome (dehydration, spinal curvature, and opportunistic infections of the eyes, genitals, or skin rash), and gross pathological changes determined by necropsies upon termination of the experiment.

Tumor measurements were obtained on an every-other-day schedule, not including weekends. Statistical significance was determined using Student's T-Test, and statistical outliers were determined by Grubb's outlier test. No outliers were detected in this study.

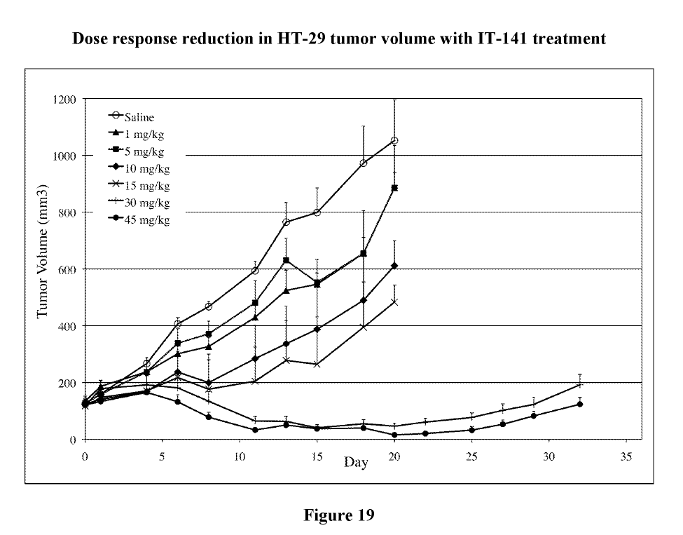

Nude mice bearing HT-29 colon tumor xenografts were treated with IT-141 (Example 19) to determine the antitumor efficacy in a dose dependent manner. Data is shown in FIG. 19 and summarized in Table 1. Treatment with 1 or 5 mg/kg IT-141 did not result in a statistically significant inhibition of tumor growth compared to control. Treatment with 10 and 15 mg/kg resulted in a 47% and 61% inhibition of tumor growth, respectively, compared to control on day 20 (p=0.032 and 0.014). Treatment with 30 mg/kg resulted in a 108% inhibition of tumor growth (p=0.004), and treatment with 45 mg/kg resulted in a 111% inhibition of tumor growth (p=0.004) compared to control at day 20. After the three injections, tumors exhibited 88% regression and 60% of the tumors exhibited complete regression.

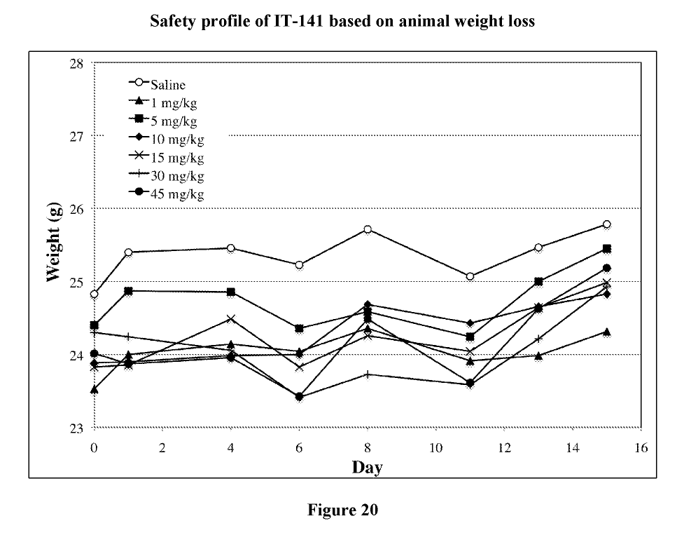

During the study the weight of each animal was recorded along with any clinical signs of toxicity. There were no treatment related deaths or observable gross toxicity in any of the treatment groups. Animal weights remained stable through the experiment, as shown in FIG. 20.

TABLE 1

| | | Tumor Volume | Standard | | |
|---|---|---|---|---|---|
| Treatment | | day 20 (mm³) | Error (mm³) | % Inhibition | P Value |
| Saline | | 1052.0 | 143.5 | 0 | ND |
| 1 | mg/kg | 886.1 | 148.7 | 18.9 | 0.230 |
| 5 | mg/kg | 828.2 | 52.2 | 24.3 | 0.079 |
| 10 | mg/kg | 612.0 | 93.6 | 47.4 | 0.032 |
| 15 | mg/kg | 483.5 | 95.5 | 60.5 | 0.014 |
| 30 | mg/kg | 46.4 | 10.0 | 107.7 | 0.004 |
| 45 | mg/kg | 15.6 | 6.2 | 111.4 | 0.004 |

### Example 31

### Antitumor Efficacy of IT-141

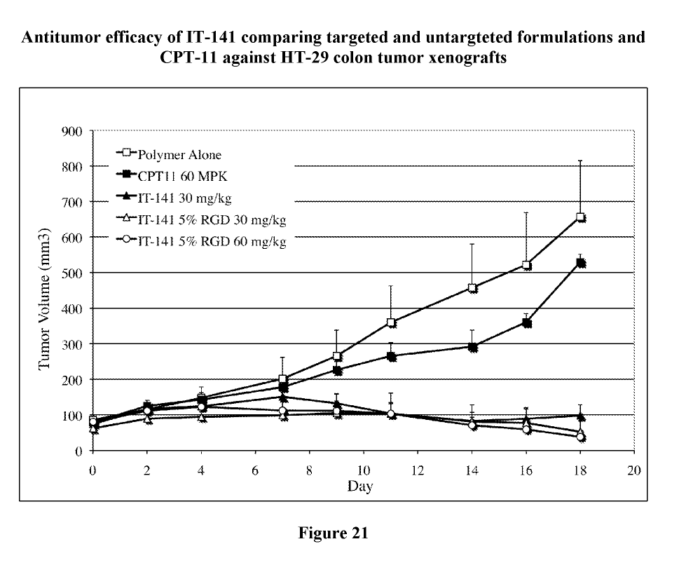

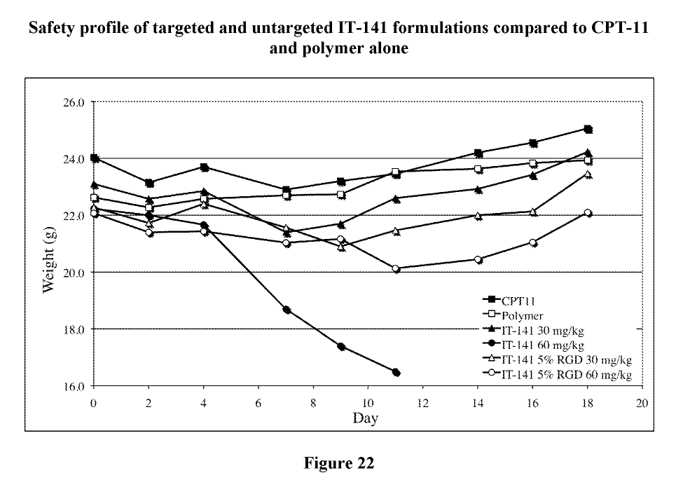

IT-141 (Example 19) dosed at 30 mg/kg resulted in 100% tumor response rate with a 32% growth rate over 18 days, and 96% inhibition of tumor growth compared to polymer alone (p=0.0056). Data is shown in FIG. 21 and summarized in Table 2. IT-141 dosed at 60 mg/kg was toxic to all animals, therefore no antitumor efficacy data was reported. Dosing with IT-141-5% RGD (5% double-cyclic RGD targeting groups, Example 20) resulted in an 83% tumor response, however tumor regression was observed over 18 days with a −15% growth rate and 102% inhibition over polymer alone (p=0.0039). Increasing the dose to 60 mg/kg resulted in 100% tumor response with a −53% growth rate and 107% inhibition compared to polymer alone (p=0.0040). CPT-11 did not significantly inhibit tumor growth compared to polymer alone. During the study the weight of each animal was recorded along with any clinical signs of toxicity. IT-141 at 60 mg/kg was toxic to all animals by day 11. One death occurred in each of the IT-141 30 mg/kg and IT-141-5% RGD 30 and 60 mg/kg groups. As shown in FIG. 22, weight loss of greater than 10% original body weight was seen for the IT-141-5% RGD 60 mg/kg group, however, recovery occurred within 1 week of the final treatment.

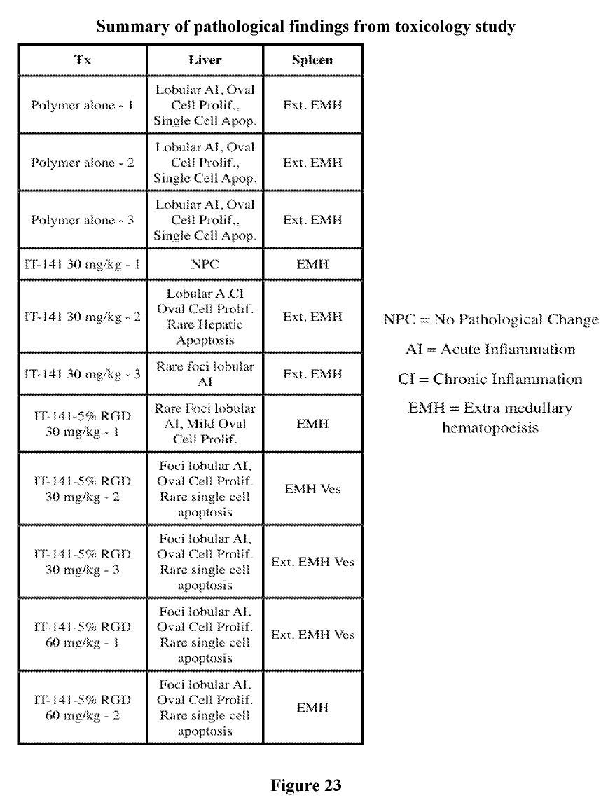

Vital organs and tumor tissues were collected on day 18 of the study for histological processing by H&E staining The summary of these findings is found in FIG. 23. Pathological analysis revealed the major toxicity of treatment to be neutropenia as determined by the presence of extramedullary hematopoiesis in the spleen. Slight liver toxicity was seen in some treatment groups, and was evident by mild lobular inflammation and oval progenitor cell proliferation in the liver samples. No treatment related toxicity was observed in kidney, heart, lung, or brain samples.

TABLE 2

| | Dose | Tumor Volume | Standard | % Inhi- | |
|---|---|---|---|---|---|
| Treatment | (mg/kg) | day 18 (mm³) | Error (mm³) | bition | P Value |
| Polymer | | 656.8 | 158.0 | 0 | ND |
| Alone | | | | | |
| CPT-11 | 60 | 529.5 | 22.2 | 21.0 | 0.211 |
| IT-141 | 30 | 98.0 | 30.0 | 95.9 | 0.006 |
| IT-141 | 30 | 52.5 | 35.6 | 101.7 | 0.004 |
| 5% RGD | | | | | |
| IT-141 | 60 | 37.9 | 9.50 | 107.4 | 0.004 |
| 5% RGD | | | | | |

### Example 32

### Antitumor Efficacy of dcRGD Targeted IT-141 Micelles

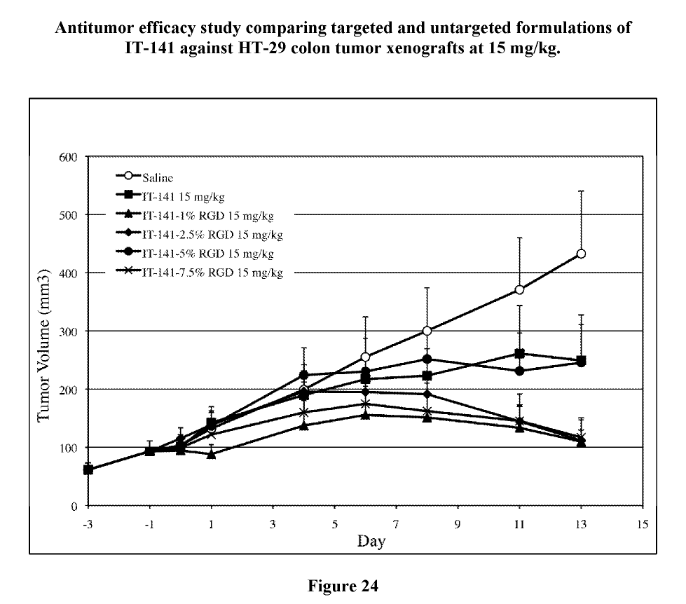

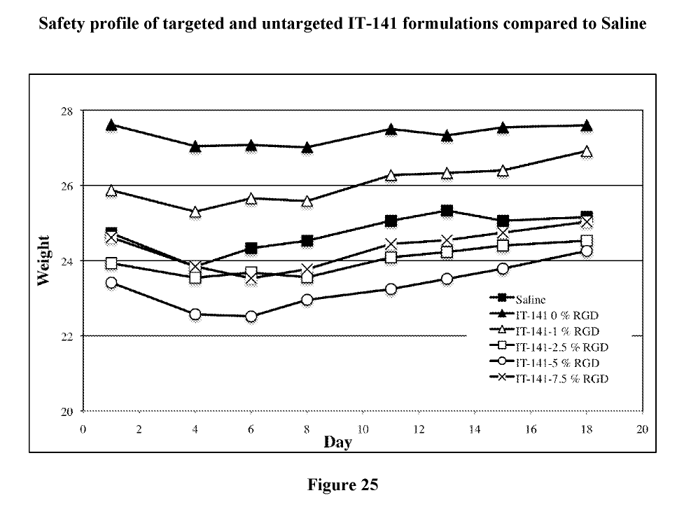

IT-141 formulations with varying per-cent coverage of RGD targeting groups (Example 20) were dosed at 15 mg/kg to mice with HT-29 tumor xenografts to determine the optimum RGD coverage for IT-141 delivery. Data are shown in FIG. 24 and summarized in Table 3. Formulations with 1%, 2.5% and 7.5% RGD all inhibited tumor growth by approximately 75% (74.6%, 74.2% and 73.0% respectively) compared to the saline group. Formulations with 0% and 5% RGD blocked tumor growth by 43.2 and 42.4%, respectively. During the study the weight of each animal was recorded along with any clinical signs of toxicity. There were no treatment related deaths or observable gross toxicity in any of the treatment groups. As seen in FIG. 25, Animal weights remained stable through the experiment.

TABLE 3

| | Dose | Tumor Volume | Standard | % Inhi- | |
|---|---|---|---|---|---|
| Treatment | (mg/kg) | day 13 (mm³) | Error (mm³) | bition | P Value |
| Saline | | 432.5 | 107.7 | 0 | ND |
| IT-141 | 15 | 249.3 | 78.2 | 42.4 | 0.081 |
| IT-141 | 15 | 109.7 | 37.7 | 74.6 | 0.009 |
| 1% RGD | | | | | |
| IT-141 | 15 | 111.6 | 18.1 | 74.2 | 0.004 |
| 2.5% RGD | | | | | |
| IT-141 | 15 | 245.6 | 65.0 | 43.2 | 0.067 |
| 5% RGD | | | | | |
| IT-141 | 15 | 116.9 | 33.7 | 73.0 | 0.005 |
| 7.5% RGD | | | | | |

### Example 33

### Antitumor Efficacy of dcRGD Targeted IT-141 Micelles

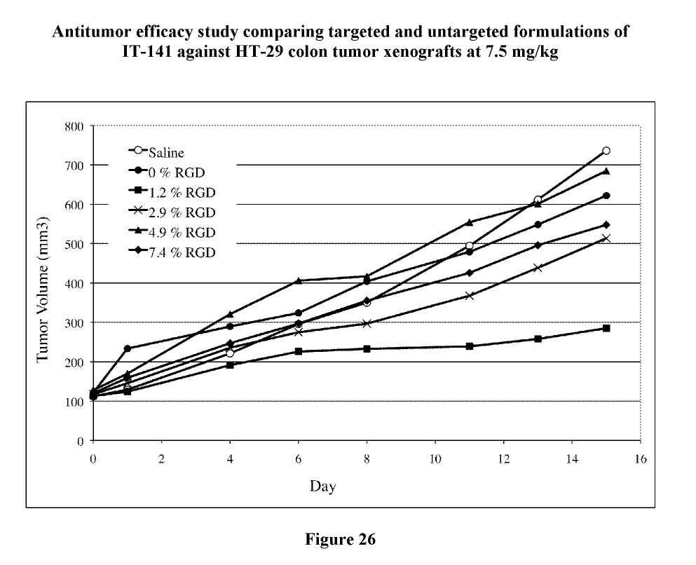

IT-141 formulations with varying per-cent coverage of RGD targeting groups (Example 20) were dosed at 7.5 mg/kg to mice with HT-29 tumor xenografts to determine the optimum RGD coverage for IT-141 delivery. Data are shown in FIG. 26 and summarized in Table 4. IT-141-1.2% RGD demonstrated significantly better efficacy at 7.5 mg/kg compared to untargeted and higher % RGD formulations. IT-141-1.2% RGD treatment resulted in 73% inhibition of tumor growth (p value=0.002) compared to saline control. This experiment shows that 1% RGD targeting exhibits the best antitumor efficacy when administered at 7.5 mg/kg.

TABLE 4

| | Dose | Tumor Volume | Standard | % Inhi- | |
|---|---|---|---|---|---|
| Treatment | (mg/kg) | day 15 (mm³) | Error (mm³) | bition | P Value |
| Saline | — | 736.2 | 121.0 | 0 | ND |
| IT-141 | 7.5 | 621.9 | 102.6 | 19.8 | 0.229 |
| IT-141 | 7.5 | 285.1 | 68.4 | 72.5 | 0.002 |
| 1.2% RGD | | | | | |
| IT-141 | 7.5 | 513.6 | 118.8 | 36.5 | 0.091 |
| 2.9% RGD | | | | | |
| IT-141 | 7.5 | 684.9 | 86.7 | 10.7 | 0.358 |
| 4.9% RGD | | | | | |
| IT-141 | 7.5 | 547.7 | 107.4 | 37.5 | 0.116 |
| 7.4% RGD | | | | | |

### Example 34

### Antitumor Efficacy of IT-141

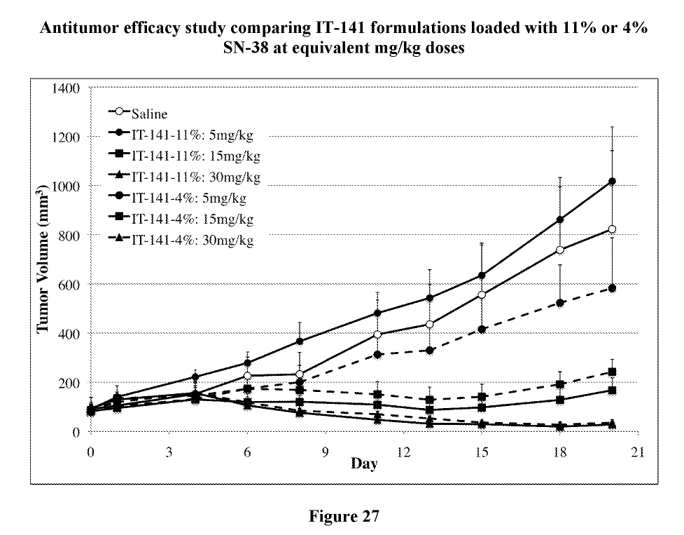

Nude mice bearing HT-29 colon tumor xenografts were dosed with IT-141 (Example 19) formulations with 11% and 4% SN-38 loading at equivalent mg/kg doses of 5, 15 and 30 mg/kg to determine if micelle drug loading effects antitumor efficacy in-vivo. Data is shown in FIG. 27 and summarized in Table 5. There were no statistical differences between the treatments at equivalent SN-38 doses. 5 mg/kg groups did not inhibit tumor growth compared to saline control. 15 mg/kg treatments inhibited tumor growth by 93% and 84% for IT-141-11% and IT-141-4%, respectively (p values=0.015 and 0.023). 30 mg/kg groups inhibited tumor growth by 112% and 110% for IT-141-11% and IT-141-4%, respectively (p values=0.014 and 0.006). All treatments were well tolerated as no treatment related deaths or gross toxicity for any of the animals occurred during this study. This study shows that the weight percentage of SN-38 in IT-141 shows no observable effect on anti-tumor efficacy in tumor bearing mice.

TABLE 5

| | Dose | Tumor Volume | Standard | % Inhi- | |
|---|---|---|---|---|---|
| Treatment | (mg/kg) | day 20 (mm³) | Error (mm³) | bition | P Value |
| Saline | — | 822.6 | 319.1 | 0 | ND |
| IT-141 11% | 5 | 1017.7 | 220.4 | 0 | 0.20 |
| IT-141 11% | 15 | 167.0 | 50.0 | 88.4 | 0.019 |
| IT-141 11% | 30 | 27.5 | 12.7 | 109.1 | 0.015 |
| IT-141 4% | 5 | 582.5 | 204.6 | 31.4 | 0.308 |
| IT-141 4% | 15 | 241.8 | 51.3 | 78.9 | 0.033 |
| IT-141 4% | 30 | 35.5 | 11.4 | 107.2 | 0.007 |

### Example 35

### Antitumor Efficacy of IT-141

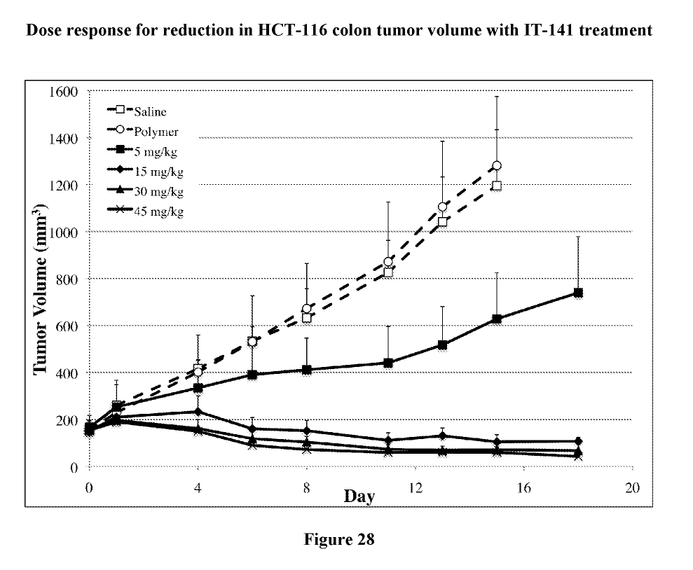

Nude mice bearing HCT-116 colon tumor xenografts were treated with IT-141 (Example 19) to determine the antitumor efficacy in a dose dependent manner. Data is shown in FIG. 28 and summarized in Table 6. Polymer alone did not inhibit tumor growth compared to saline control. Treatment with 5 mg/kg IT-141 resulted in 59% inhibition of tumor growth compared to polymer alone (p value=0.008). Treatments with 15 mg/kg, 30 mg/kg and 45 mg/kg all resulted in tumor regression compared to polymer alone, with treatments resulting in 104, 107 and 108% inhibition, respectively (p values=0.00004, 0.00003 and 0.0001). After the three injections, tumor regression of 73% was observed for the 30 mg/kg dosage group. This, along with Example 34, demonstrates the efficacy of IT-141 across multiple xenograft models.

TABLE 6

| | Dose | Tumor Volume | Standard | % Inhi- | |
|---|---|---|---|---|---|
| Treatment | (mg/kg) | day 15 (mm³) | Error (mm³) | bition | P Value |
| Saline | — | 1195.3 | 379.1 | ND | ND |
| Polymer | — | 1281.4 | 151.9 | 0 | ND |
| Alone | | | | | |
| IT-141 | 5 | 628.0 | 196.3 | 59.1 | 0.008 |
| IT-141 | 15 | 104.8 | 29.8 | 104.1 | 0.00004 |
| IT-141 | 30 | 71.0 | 15.7 | 107.3 | 0.00003 |
| IT-141 | 45 | 59.8 | 7.4 | 108.3 | 0.00016 |

### Example 36

### Antitumor Efficacy of dcRGD Targeted IT-141

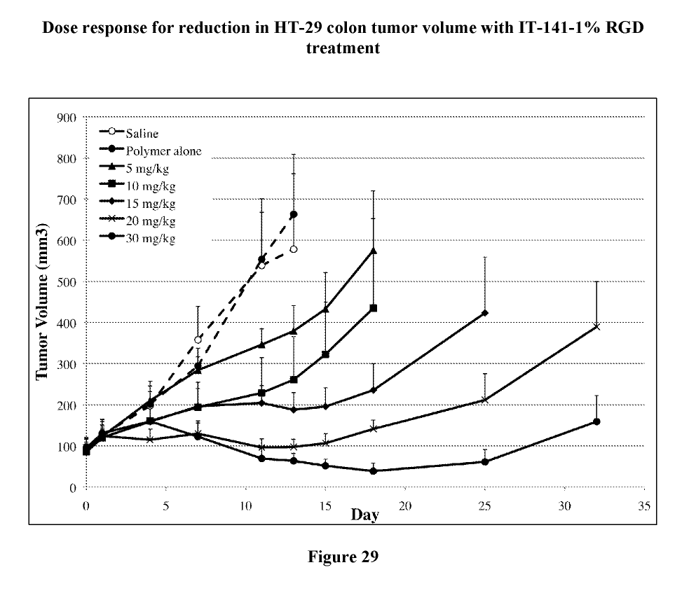

Nude mice bearing HT-29 colon tumor xenografts were treated with IT-141-1% RGD (Example 20) to determine the antitumor efficacy in a dose dependent manner. Data is shown in FIG. 29 and summarized in Table 7. Polymer alone did not inhibit tumor growth compared to saline control. Treatment with 5 mg/kg, 10 mg/kg, and 15 mg/kg IT-141-1% RGD resulted in 41%, 59% and 81% inhibition, respectively (p values=0.07, 0.03, and 0.002). Treatment with 20 mg/kg induces tumor stasis, resulting in 98% inhibition of tumor growth (p value=0.0004). Treatment with 30 mg/kg induced tumor regression, resulting in 108% inhibition of tumor growth compared to polymer alone (p value=0.0002). As shown in this study, the antitumor efficacy of IT-141-1% is dose-dependent.

TABLE 7

| | Dose | Tumor Volume | Standard | % Inhi- | |
|---|---|---|---|---|---|
| Treatment | (mg/kg) | day 15 (mm³) | Error (mm³) | bition | P Value |
| Saline | — | 578.1 | 183.7 | ND | ND |
| Polymer | — | 662.9 | 145.8 | 0 | ND |
| Alone | | | | | |
| IT-141 | 5 | 432.9 | 88.6 | 40.5 | 0.072 |
| 1% RGD | | | | | |
| IT-141 | 10 | 322.5 | 127.0 | 59.0 | 0.033 |
| 1% RGD | | | | | |
| IT-141 | 15 | 195.7 | 45.8 | 80.7 | 0.002 |
| 1% RGD | | | | | |
| IT-141 | 20 | 106.5 | 23.2 | 98.1 | 0.0004 |
| 1% RGD | | | | | |
| IT-141 | 30 | 51.9 | 16.2 | 107.5 | 0.0002 |
| 1% RGD | | | | | |

### Example 37

### Pharmacokinetics of IT-141

Pharmacokinetic and biodistribution data was generated from mice carrying HT-29 xenografts. IT-141 (Example 19) was administered by a fast IV bolus into the tail vein and plasma and organs were collected by cardiac puncture at times of 5 and 15 minutes, 1, 4, 12, 24, 48, and 72 hours with three mice utilized for each time point.

Plasma samples were prepared for quantitation by HPLC-FLD in the following manner: 50 μL plasma was vortexed for 10 minutes with 150 μL of extraction solution (1% perchloric acid in methanol with ˜1.2 μg/mL camptothecin as an internal standard). After vortexing, the samples were centrifuged at 13.2K RPM at 4° C. for 10 minutes. 150 μL of the supernatant was transferred to a HPLC vial for analysis. 7 calibration standards and 6 controls were prepared by mixing 5 μL of a known concentration of IT-141 in water with 45 μL blank plasma and vortexing for 10 minutes. 150 μL of extraction solution was then added and vortexed for an additional 10 minutes. The samples were centrifuged at 13.2 K RPM for at 4° C. for 10 minutes. 150 μL of the supernatant was transferred to a HPLC vial for analysis. 20 μL sample injections were made onto a Grace LiChrosphere RP Select B 5 μm 4.6×250 mm HPLC column and SN-38 was detected by fluorescence detection (355 nm ex; 515 nm em). The mobile phase was 70% buffer and 30% acetonitrile (buffer=10 mM sodium phosphate with 0.1% triethyl amine adjusted to pH 3.5) flowing at 0.8 mL/min with an 18 minute run time. A calibration curve was constructed from the area under the curve of each of the seven standards and the SN-38 of each of the unknown samples determined from the curve. All control injections exhibited less than 10% deviation from the known value.

Liver samples were prepared for quantitation by HPLC-FLD in the following manner: Livers were weighed and diluted 5:1 (mL buffer to g liver) with 20 mM ammonium acetate at pH 3.5. 50 μL of homogenate was vortexed for 10 minutes with 150 μL of extraction solution (1% perchloric acid in methanol with ˜1.2 μg/mL camptothecin as an internal standard). After vortexing, the samples were centrifuged at 13.2K RPM at 4° C. for 10 minutes. 150 μL of the supernatant was transferred to a HPLC vial for analysis. 7 calibration standards and 6 controls were prepared by mixing 5 μL of a known concentration of IT-141 in water with 45 μL blank homogenate and vortexing for 10 minutes. 150 μL of extraction solution was then added and vortexed for an additional 10 minutes. The samples were centrifuged at 13.2K RPM for at 4° C. for 10 minutes. 150 μL of the supernatant was transferred to a HPLC vial for analysis. Tumors were homogenized and prepared in a matter identical to the liver samples. HPLC conditions were identical to those used for the plasma.

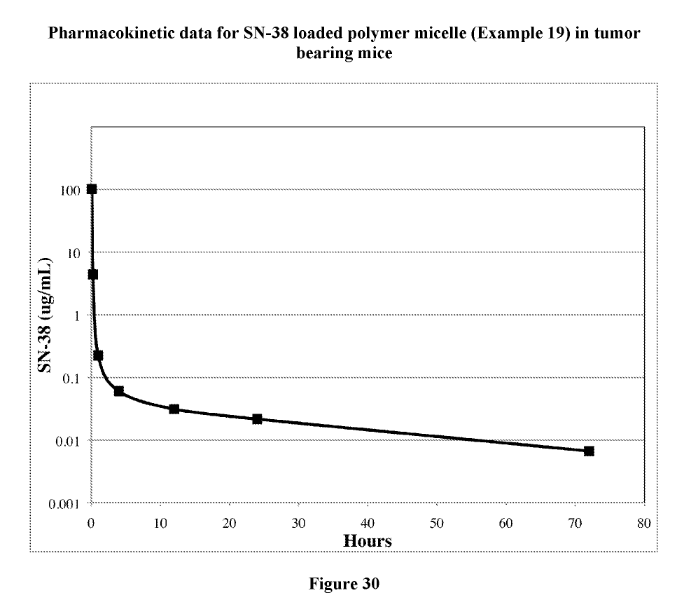

FIG. 30 shows the SN-38 concentration in plasma collected from HT-29 tumor bearing mice over 72 hours. Analysis of the plasma concentration vs. time curve resulted in the following pharmacokinetic parameters: a CMax of 102 μg/mL at a TMax of 5 minutes, an area under to curve of 12.4 hours*μg SN-38/mL, and an overall half-life of 8.7 hours.

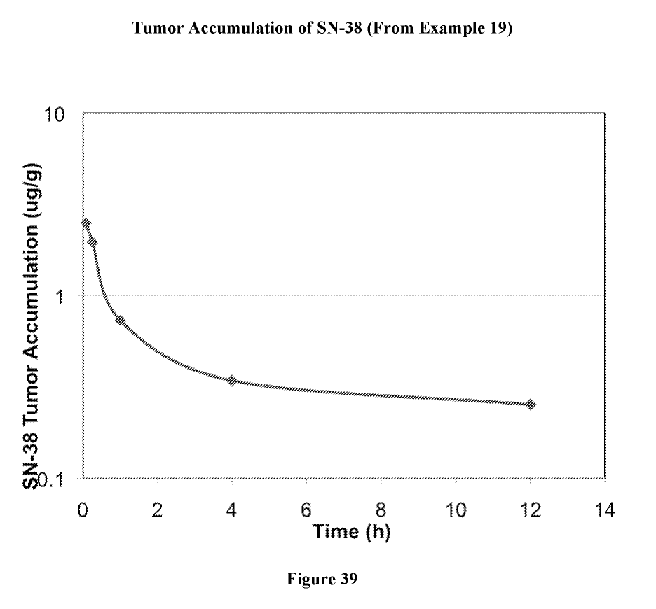

FIG. 39 shows the SN-38 concentration in tumors collected from HT-29 tumor bearing mice over 72 hours. Analysis of the tumor concentration vs. time curve resulted in the following pharmacokinetic parameters: a CMax of 2.5 μg/mL at a TMax of 5 minutes, an area under to curve of 7.5 hours*μg SN-38/mL, and an overall half-life of 5.9 hours.

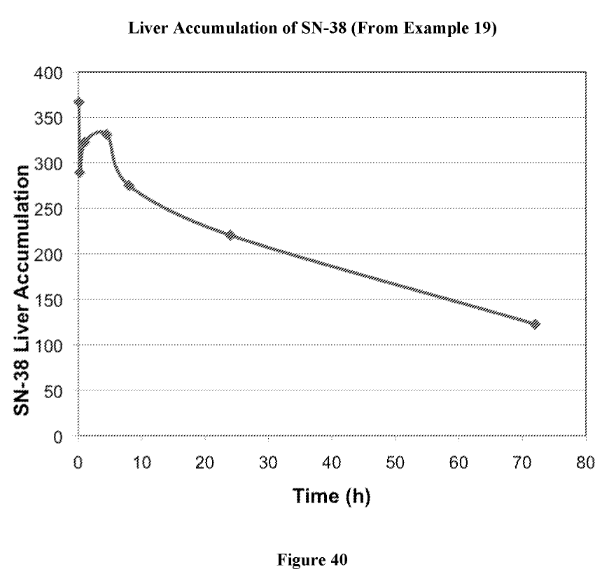

FIG. 40 shows the SN-38 concentration in livers collected from HT-29 tumor bearing mice over 72 hours. Analysis of the liver concentration vs. time curve resulted in the following pharmacokinetic parameters: a CMax of 366.8 μg/mL at a TMax of 5 minutes, an area under to curve of 14675.9 hours*μg SN-38/mL, and an overall half-life of 50.1 hours.

### Example 38

### MTD Study of Empty Micelles in CD-1 Mice

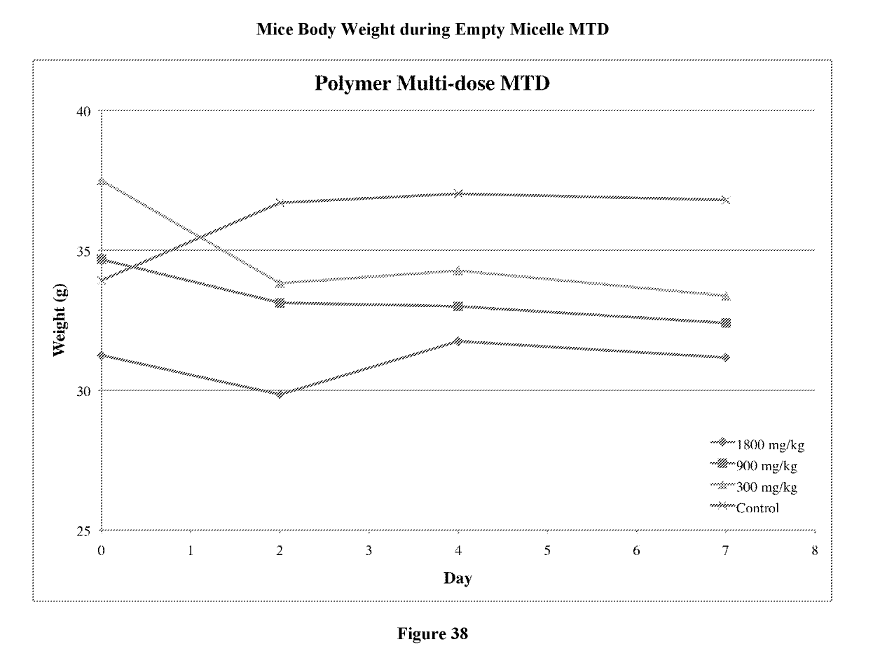

CD-1 mice were separated into groups four groups of six mice each and were administered a micelle comprised of Formula I alone (without SN-38) at doses of 0, 300, 900 or 1,800 mg/kg. The dosage schedule was every fourth day for three injections. The weights were recorded every other day for ten days, and are reported in FIG. 38. All mice remained healthy throughout the study and a maximum tolerated dose was found to be above 1,800 mg/kg.

### Example 39

### Solid Phase Synthesis of Alkyne Functionalized Targeting Groups

The alkyne functionalized targeting groups utilized in Example 12, Example 13, Example 14, and Example 15 were prepared using an ABI peptide synthesizer. Peptides were grown from the N-terminus using standard FMoc chemistry from a Merrifield resin, capped with 5-pentynoic acid, then deprotected after cleavage from the resin. The individual peptides were purified by prep HPLC and characterized by mass spectrometry. In the case of Example 12, the disulfide linkages were prepared by dissolving the peptide in water at a concetration of ˜1 mM, then stirring 3 hours. The cyclized peptide was isolated by lyophilization.

### Example 40

### Preparation of IT-141: SN-38 Encapsulation with Microfluidizer

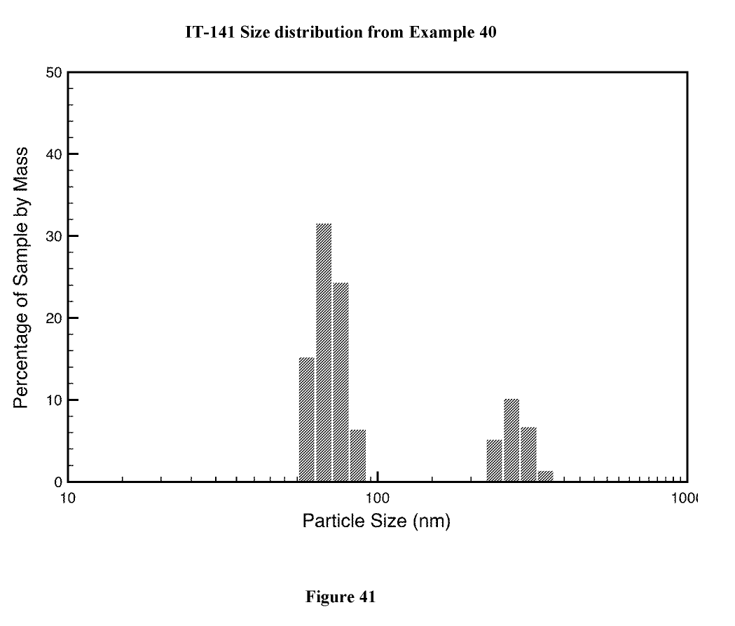

N₃-Poly(ethylene oxide)₂₇₀-b-Poly(Asp₁₀)-b-Poly(dLeu₂₀-co-Tyr₂₀)-Ac (1.5 g) from Example 11 and sucrose (2 g) was dissolved in water (200 mL). SN-38 (113 mg) was weighed into a 20 ml vial then dissolved in DMSO (2 mL). Once homogeneous, the DMSO solution was diluted with toluene (8 mL). The aqueous polymer solution was stirred with Silverson high shear mixer equipped with a fine emulsion screen. The mixer was turned to 10,000 rpm and the solution stirred. The organic solution was then added drop-wise to the reaction flask, resulting in a milk-like emulsion. The solution was mixed for 1 minute then transferred to a microfluidizer. The microfluidizer (Microfluidics M-110Y equipped with a Y interaction chamber with no auxillary interaction chamber.) The solution was processed through the microfluidizer for three passes at 120 psi. The resulting solution was allowed to stir at room temperature for 12 hours in a fume hood. The solution was transferred to a dialysis bag (3500 MWCO) and dialazyled against a 1% aqueous sucrose solution (2 L). After 16 hours, the solution was filtered through a 0.22 μm PES membrane. A yellow powder (2.8 g, 77% yield) was recovered after lyophilization. HPLC showed that the yellow powder contained 2.72% SN-38 by weight for a loading efficiency of 90%. The resulting micelle diameter was 105 nm by DLS (Gaussian fit). DLS histogram is shown in FIG. 41.

### Example 41

### Pharmacokinetics and Biodistribution of IT-141

Pharmacokinetic and biodistribution data was generated from mice carrying HT-29 xenografts. IT-141 (Example 40) was administered by a fast IV bolus into the tail vein and plasma and organs (liver, tumor, and spleen) were collected by cardiac puncture at times of 5 and 15 minutes, 1, 4, 12, 24, 48, and 72 hours with eight mice utilized for each time point.

Plasma samples were prepared for quantitation by HPLC-FLD in the following manner: 50 μL plasma was vortexed for 10 minutes with 150 μL of extraction solution (1% perchloric acid in methanol with ˜1.2 μg/mL camptothecin as an internal standard). After vortexing, the samples were centrifuged at 13.2K RPM at 4° C. for 10 minutes. 150 μL of the supernatant was transferred to a HPLC vial for analysis. 7 calibration standards and 6 controls were prepared by mixing 5 μL of a known concentration of IT-141 in water with 45 μL blank plasma and vortexing for 10 minutes. 150 μL of extraction solution was then added and vortexed for an additional 10 minutes. The samples were centrifuged at 13.2K RPM for at 4° C. for 10 minutes. 150 μL of the supernatant was transferred to a HPLC vial for analysis. 20 μL sample injections were made onto a Grace LiChrosphere RP Select B 5 μm 4.6×250 mm HPLC column and SN-38 was detected by fluorescence detection (355 nm ex; 515 nm em). The mobile phase was 70% buffer and 30% acetonitrile (buffer=10 mM sodium phosphate with 0.1% triethyl amine adjusted to pH 3.5) flowing at 0.8 mL/min with an 18 minute run time. A calibration curve was constructed from the area under the curve of each of the seven standards and the SN-38 of each of the unknown samples determined from the curve. All control injections exhibited less than 10% deviation from the known value.

Liver samples were prepared for quantitation by HPLC-FLD in the following manner: Livers were weighed and diluted 5:1 (mL buffer to g liver) with 20 mM ammonium acetate at pH 3.5. 50 μL of homogenate was vortexed for 10 minutes with 150 μL of extraction solution (1% perchloric acid in methanol with ˜1.2 μg/mL camptothecin as an internal standard). After vortexing, the samples were centrifuged at 13.2K RPM at 4° C. for 10 minutes. 150 μL of the supernatant was transferred to a HPLC vial for analysis. 7 calibration standards and 6 controls were prepared by mixing 5 μL of a known concentration of IT-141 in water with 45 μL blank homogenate and vortexing for 10 minutes. 150 μL of extraction solution was then added and vortexed for an additional 10 minutes. The samples were centrifuged at 13.2K RPM for at 4° C. for 10 minutes. 150 μL of the supernatant was transferred to a HPLC vial for analysis. Tumors were homogenized and prepared in a matter identical to the liver samples. HPLC conditions were identical to those used for the plasma.

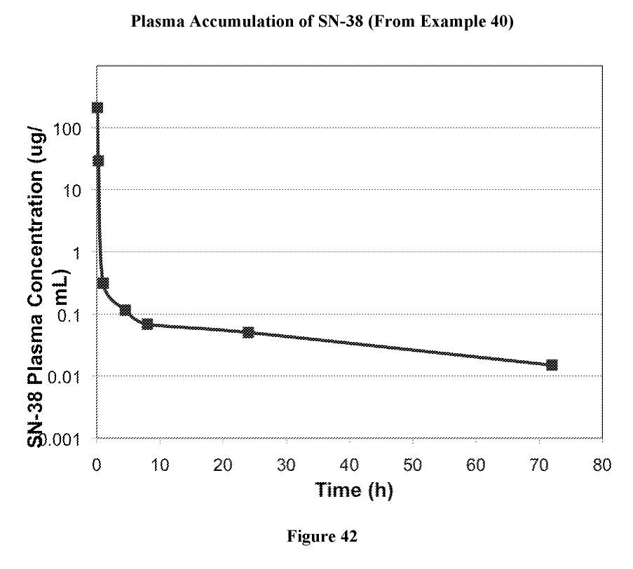

FIG. 42 shows the SN-38 concentration in plasma collected from HT-29 tumor bearing mice over 72 hours. Analysis of the plasma concentration vs. time curve resulted in the following pharmacokinetic parameters: a CMax of 209.5 μg/mL at a TMax of 5 minutes, an area under to curve of 34.6 hours*μg SN-38/mL, and an overall half-life of 8.5 hours.

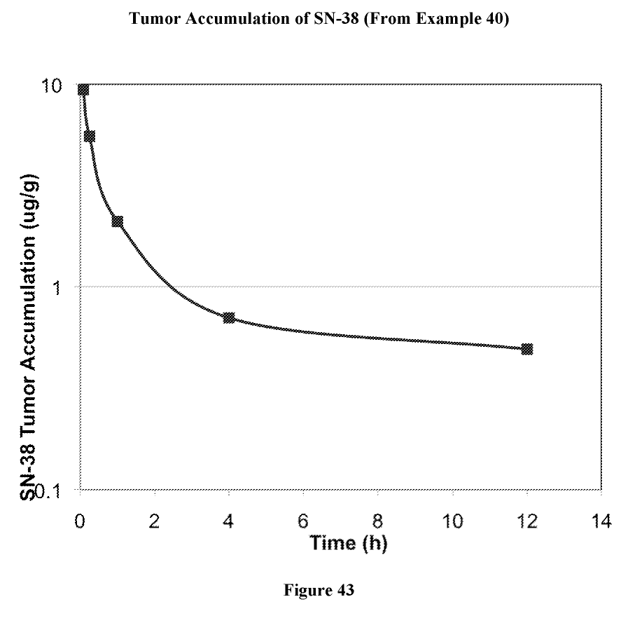

FIG. 43 shows the SN-38 concentration in tumors collected from HT-29 tumor bearing mice over 72 hours. Analysis of the tumor concentration vs. time curve resulted in the following pharmacokinetic parameters: a CMax of 9.4 μg/mL at a TMax of 5 minutes, an area under to curve of 16.4 hours*μg SN-38/mL, and an overall half-life of 3.9 hours.

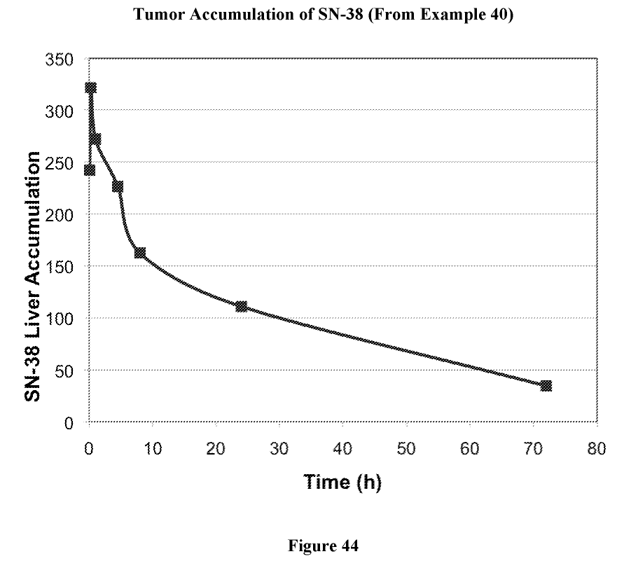

FIG. 44 shows the SN-38 concentration in livers collected from HT-29 tumor bearing mice over 72 hours. Analysis of the liver concentration vs. time curve resulted in the following pharmacokinetic parameters: a CMax of 321.7 μg/mL at a TMax of 15 minutes, an area under to curve of 7498.3 hours*μg SN-38/mL, and an overall half-life of 24.1 hours.

### Example 42

### Pharmacokinetics of IT-141 in Cannulated Rats

Pharmacokinetic data was generated from cannulated rats. IT-141 (Example 40) or irinotecan at 5 mg/kg (SN-38 equivalent mass) was administered by a fast IV bolus into catherized jugular vein and plasma was collected via the jugular vein catheter at times of 1, 5, 15, 30, 60 and 240 minutes and placed into potassium-EDTA vials and snap frozen with dry ice. Each group consisted of three rats.

Plasma samples were prepared for quantitation by HPLC-FLD in the following manner: 50 μL plasma was vortexed for 10 minutes with 150 μL of extraction solution (1% perchloric acid in methanol with ˜1.2 μg/mL camptothecin as an internal standard). After vortexing, the samples were centrifuged at 13.2K RPM at 4° C. for 10 minutes. 150 μL of the supernatant was transferred to a HPLC vial for analysis. 7 calibration standards and 6 controls were prepared by mixing 5 μL of a known concentration of IT-141 in water with 45 μL blank plasma and vortexing for 10 minutes. 150 μL of extraction solution was then added and vortexed for an additional 10 minutes. The samples were centrifuged at 13.2K RPM for at 4° C. for 10 minutes. 150 μL of the supernatant was transferred to a HPLC vial for analysis. 20 μL sample injections were made onto a Grace LiChrosphere RP Select B 5 μm 4.6×250 mm HPLC column and SN-38 was detected by fluorescence detection (355 nm ex; 515 nm em). The mobile phase was 70% buffer and 30% acetonitrile (buffer=10 mM sodium phosphate with 0.1% triethyl amine adjusted to pH 3.5) flowing at 0.8 mL/min with an 18 minute run time. A calibration curve was constructed from the area under the curve of each of the seven standards and the SN-38 of each of the unknown samples determined from the curve. All control injections exhibited less than 10% deviation from the known value.

Table 8 shows the summary of the data generated from the rat PK experiment. Noteably, the Cmax of IT-141 is 41.2 μg/mL vs. 0.35 μg/mL for irinotecan. The AUC for IT-141 was 3.28 hours*μg SN-38/mL vs. 0.26 hours*μg SN-38/mL for irinotecan.

TABLE 8

| | CMax | AUC (hr) | TermT½ |
|---|---|---|---|
| Irinotecan | 0.35 | 0.26 | 2.03 |
| IT-141 | 41.2 | 3.28 | 7.49 |

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

We claim:

1. A micelle, having SN-38 encapsulated therein, comprising a multiblock copolymer of formula I: 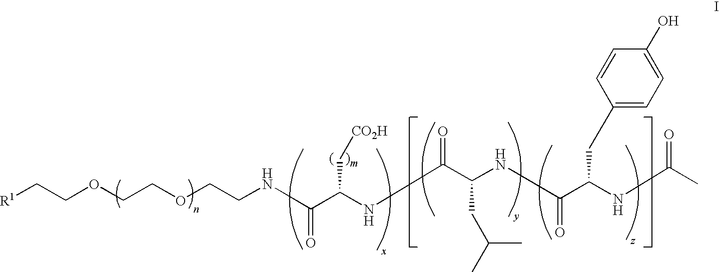
wherein:
R¹ is —OCH₃, —N₃, or 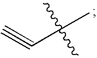
n is 110 to 450;
m is 1 or 2;
x is 3 to 50;
y is 5 to 50; and
z is 5 to 50.

2. The micelle according to claim 1 wherein R¹ is —OCH₃.

3. The micelle according to claim 2 wherein: n is about 270; m is 1; x is about 10; y is about 20; and z is about 20.

4. The micelle according to claim 2 wherein: y is 20±5 and z is 20±5.

5. The micelle according to claim 1 wherein, wherein R¹ is —N₃.

6. The micelle according to claim 5 wherein: n is about 270; m is 1; x is about 10; y is about 20; and z is about 20.

7. A crosslinked micelle, having SN-38 encapsulated therein, comprising a crosslinked multiblock polymer of formula X: 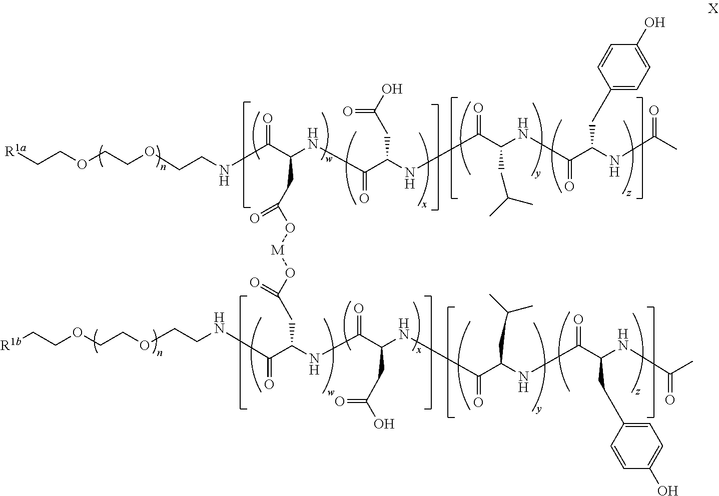
wherein:
R^{1a} and R^{1b} are independently selected from —OCH₃, —N₃, 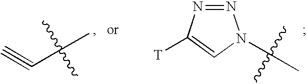
T is a targeting group moiety;
M is a suitable metal ion;
n is 110 to 450;
w is 3 to 50;
x is 0 to 50, provided that the sum of w and x is no more than 50;
y is 5 to 50; and
z is 5 to 50.

8. The micelle according to claim 7 wherein R^{1a} and R^{1b} are simultaneously —OCH₃.

9. The micelle according to claim 8 wherein: n is about 270; m is 1; x is about 10; y is about 20; and z is about 20.

10. The micelle according to claim 7 wherein R^{1a} and R^{1b} are simultaneously —N₃.

11. The micelle according to claim 10 wherein: n is about 270; m is 1; x is about 10; y is about 20; and z is about 20.

12. The micelle according to claim 7 wherein R^{1a} is —OCH₃ and R^{1b} is

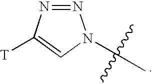

13. The micelle according to claim 7, wherein R^{1a} is —OCH₃ and R^{1b} is —OCH₃.

14. The micelle according to claim 7, wherein R^{1a} is —OCH₃ and R^{1b} is —N₃.

15. The micelle according to claim 12, wherein T is RGD or Her-2.
